(19) United States
(12) Patent Application Publication
(10) Pub. No.: US 2026/0263162 A1
(43) Pub. Date: Sep. 10, 2026

(54) CAMERA NAVIGATION SYSTEM

(71) Applicant: Applied Medical Resources Corporation, Rancho Santa Margarita, CA (US)

(72) Inventors: Nathan Landino, Rancho Santa Margarita, CA (US); Aditya Kumakale, Rancho Santa Margarita, CA (US); Irfan Kil, Rancho Santa Margarita, CA (US); Branden Carter, Rancho Santa Margarita, CA (US); Ramin Rahgozar, Rancho Santa Margarita, CA (US); Chia Yu Chiang, Rancho Santa Margarita, CA (US); Zachary Marin, Rancho Santa Margarita, CA (US); Lindsey Chase, Rancho Santa Margarita, CA (US); Christopher Boyer, Rancho Santa Margarita, CA (US)

(21) Appl. No.: 19/575,130

(22) Filed: Mar. 23, 2026

Related U.S. Application Data
(63) Continuation of application No. PCT/US2024/048724, filed on Sep. 26, 2024.
(60) Provisional application No. 63/587,009, filed on Sep. 29, 2023.
(60) Provisional application No. 63/571,243, filed on Mar. 28, 2024.

(51) Int. Cl. A61B 34/20; A61B 1/00; A61B 1/313; A61B 34/10

(52) U.S. Cl. CPC A61B 34/20; A61B 1/00006; A61B 1/00045; A61B 1/00055; A61B 1/3132; A61B 34/10; A61B 2034/102; A61B 2034/2055

(57) ABSTRACT

A camera navigation system is provided. The camera navigation system includes an insert or grid having a plurality of markers configured to assist in the tracking of a simulated laparoscope. The camera navigation system generates a digital environment with computer-generated elements for different camera navigation exercises that are displayed on a monitor for a user to view. The position of the simulated laparoscope with respect to the training environment is simulated within the digital environment as a cursor and movements of the simulated laparoscope with respect to the training environment correspond to movements of the cursor within the digital environment. The camera navigation system is compatible with both zero degree and angled laparoscopes and has camera navigation exercises that are provided to train users on different camera navigation skills. Feedback is also provided characterizing the user's performance of the camera navigation exercises.

### CROSS-REFERENCE TO RELATED APPLICATION

This is a continuation of International Application No. PCT/US2024/048724, filed on Sep. 26, 2024, which claims priority to and benefit of U.S. Provisional Patent Application Ser. No. 63/587,009 entitled “Camera Navigation System” filed on Sep. 29, 2023 and U.S. Provisional Patent Application Ser. No. 63/571,243 entitled “Camera Navigation System” filed on Mar. 28, 2024, all the disclosures of which are incorporated herein by reference in their entirety.

### BACKGROUND

This application relates to surgical training, and in particular, to devices and methods for training camera navigation skills in a laparoscopic and a digitally generated environment.

During laparoscopic or endoscopic surgical procedures, someone manipulates a laparoscope or endoscope to provide a view within a patient. The view is displayed on a nearby video display screen or monitor. In various situations, the laparoscope or endoscope can be controlled by someone other than the surgeon performing the laparoscopic or endoscopic surgical procedure. For example, a medical student or intern can be tasked with navigating the laparoscope or endoscope and must quickly learn skills necessary for providing optimal visibility such as recognizing and centering the operative field, maintaining the correct horizontal axis, knowing when to zoom in or out, holding a steady image, and tracking the surgical instruments being used by the surgeon while the instruments are in motion. An experienced camera operator who knows the case well enough can predict the next moves of the surgeon and move the laparoscope or endoscope accordingly. Thus, camera navigation is crucial to the proper execution of laparoscopic or endoscopic surgical procedures. As such, one important part of laparoscopic or endoscopic camera navigation skills training is to provide the ability to train complex camera movements for the purposes of following the movements of the surgeon that is performing the surgical procedure all while overcoming the difficulties outlined above.

Although camera movements may vary depending on the specific surgical procedure being performed, a simple and universal method of training and assessing camera navigation skills is sought. Some studies have started investigating the impact of poor camera navigation in a surgical case, predicting that suboptimal imaging can lead to surgeon frustration and inefficiency. The studies also indicate that the flow of the operation can be seriously disrupted when the surgeon must stop operating due to poor visualization of the surgical field. Furthermore, delays arising from poor visualization during the surgical procedure will also increase time in the operating room.

Not only do new practitioners have to learn camera navigational skills but there is also a need for those who have some training to 1) obtain further practice/polish already learned camera navigation stills or 2) learn and practice new camera navigation skills associated that are unique to newly introduced surgical procedures.

While training can be acquired in the operating room, there is an increasing interest in devising faster and more efficient training methods, preferably outside the operating room. Surgeons that attain a reasonable level of skills outside the operating room are better prepared when they enter the operating room. This allows the surgeons the ability to obtain valuable operating room-related experience that can be optimized thereby lowering the risk to patients and as well as reducing costs. Thus, there is a need for a specialized camera navigation system that has exercises directed towards learning and training camera navigation skills useful for assisting surgeons in the surgical procedures. Such an exercise tool would allow trainees to gain the camera navigation skills necessary for providing the best visibility for the surgeon prior to entering the operating room.

### SUMMARY

In accordance with various embodiments of the present invention, a camera navigation system is provided that includes a simulated laparoscope having a camera, a training environment having markers that are provided to track a position of the simulated laparoscope, a scope view generator, and/or a monitor that displays the digital environment and the feedback for the camera navigation exercises. The scope view generator is provided and is able to generate a digital environment that corresponds to the training environment and has computer-generated elements, track a position of the simulated laparoscope with respect to the training environment by processing image data obtained from the simulated laparoscope which contains markers at regular time intervals, calculates the positional information of the simulated laparoscope from the image data, updates the digital environment using the positional information, monitors user performance during a camera navigation exercise by comparing the positional information of the simulated laparoscope with the computer-generated elements within the digital environment, and generates feedback based on the user performance of the camera navigation exercise.

In accordance with various embodiments of the present invention, a device for tracking a location of a simulated laparoscope is provided. The device includes the simulated laparoscope with a camera that will be tracked, a training environment that includes an insert or grid having many unique markers, and/or a scope view generator that is used to determine a position of the simulated laparoscope. The scope view generator comprises a memory storage device that stores locations of each of the unique markers, an executable application, e.g., a computer vision application, that is able to identify what unique markers are captured in the image data and determine the positional information of the simulated laparoscope based on the markers identified, and a scope view generator that is able to generate the digital environment and computer-generated elements incorporated into the digital environment.

In accordance with various embodiments of the present invention a camera navigation system is provided. The camera navigation system is used with a simulated angled laparoscope that has a camera and rotary sensor. The camera navigation system also has a training environment that contains markers used to track a position of the simulated angled laparoscope. A scope view generator is also included that is provided to generate a digital environment that corresponds to the training environment and contains computer-generated elements based on a camera navigation exercises selected by a user. The scope view generator tracks and determines the position of the simulated laparoscope which is based on the image data and measured angled from the rotary sensor. The scope view generator updates the digital environment using the positional information of the simulated laparoscope and monitors the user's performance of the camera navigation exercise. The scope view generator is able to detect whether a collision is present between a cursor (the digital environment representation of the position of the simulated laparoscope) and one or more computer-generated elements (such as a target).

In accordance with various embodiments of the present invention an insert or grid for tracking a simulated laparoscope is provided. The insert or grid has markers which are unique from one another. The markers are also arranged in a pre-determined patterned arrangement over a pre-defined space. The position of the simulated laparoscope is determinable based on what markers are captured. The information about the locations of the markers on the insert or grid are stored in memory and retrievable by a scope view generator when determining the position of the simulated laparoscope.

In accordance with various embodiments of the present invention a device for tracking a location of a simulated angled laparoscope is provided. The device includes a simulated angled laparoscope that has a camera and a sensor designed to detect a rotation of the simulated angled laparoscope. The device also includes a training environment that includes an insert or grid that has many unique markers arranged thereon. The device also includes a scope view generator that is provided to identify a position of the simulated angled laparoscope, the scope view generator including memory that stores each of the locations of the unique markers, an executable application, e.g., a computer vision application, that identifies the unique markers captured as image data by the simulated angled laparoscope and determines the position of the simulated angled laparoscope from the image data and an angular rotation from the sensor. The device also includes a scope view generator that generates a digital environment that corresponds to the training environment and computer-generated elements incorporated into the digital environment.

### BRIEF DESCRIPTION OF THE DRAWINGS

The present invention maybe better understood taken in connection with the accompanying drawings in which the referenced numerals designate like parts throughout the figures thereof.

FIG. 1A-F IG. 1G-2 illustrate various embodiments of a camera navigation box and portions thereof.

FIG. 2A-F IG. 2C illustrate various embodiments of a camera navigation system.

FIG. 3 illustrates an embodiment of an insert or grid having a plurality of markers.

FIG. 4A-F IG. 4C illustrates various embodiments of computer-generated menu or portions thereof for the camera navigation system.

FIG. 5A-F IG. 9 illustrate various embodiments of feedback generated by the camera navigation system that is displayed within the digital environment.

FIG. 10A-F IG. 10B illustrate exemplary calculations for simulated laparoscope rotations performed by the camera navigation system.

FIG. 11 illustrates an exemplary embodiment of a meter within the digital environment used to quantify a user's performance.

FIG. 12 illustrates an exemplary calculation for viewing distance between a simulated laparoscope and a target.

FIG. 13 illustrates an exemplary embodiment of a range meter.

FIG. 14A and FIG. 14B illustrate an exemplary embodiment of a trace camera navigation exercise.

FIG. 15 illustrates an exemplary calculation for determining proficiency in the trace camera navigation exercise.

FIG. 16A and FIG. 16B illustrate an exemplary embodiment of a follow camera navigation exercise.

FIG. 17A and FIG. 17B illustrate an exemplary embodiment of a framing camera navigation exercise.

FIG. 18A-F IG. 18E-3 illustrate exemplary camera navigation exercises for a simulated angled laparoscope.

FIG. 19 illustrates an exemplary embodiment of the camera navigation system.

FIG. 20 illustrates an exemplary RGB conversion to greyscale.

FIG. 21 illustrates an exemplary binary image.

FIG. 22 illustrates an exemplary contour calculation.

FIG. 23 illustrates an exemplary filtering for quadrilateral shapes.

FIG. 24A-F IG. 24C illustrate an exemplary filtering using corners of a quadrilateral shape.

FIG. 25 illustrates an exemplary transformation matrix for determining distortion.

FIG. 26 illustrates an exemplary step of adding corner points.

FIG. 27 illustrates an exemplary step of labeling each of the corners.

FIG. 28 illustrates an exemplary step of reprojecting the corner points with identified corners.

FIG. 29 illustrates an exemplary embodiment of the camera navigation system.

FIG. 30 illustrates an exemplary surgical trainer.

FIG. 31 illustrates portions of an exemplary simulated angled laparoscope

### DETAILED DESCRIPTION

In accordance with various embodiments, a camera navigation system is provided. The camera navigation system is designed to train users in various camera navigation-related skills. Users are also able to learn and practice using different types of simulated laparoscopes, for example zero degree and angled laparoscopes. To simulate actual surgical conditions whereby a user (e.g., a surgeon) maneuvers a laparoscope within a patient during a surgical procedure, the present camera navigation system is designed to have a training environment that is housed within a camera navigation box and/or surgical trainer. The simulated laparoscope is used to capture image data from the training environment. In various embodiments, a scope view generator is provided which utilizes the captured image data from the simulated laparoscope to determine current positional data of the simulated laparoscope with respect to the training environment. In addition, the scope view generator is configured to generate a digital environment and corresponding computer-generated elements that utilizes the captured image data. The generated digital environment is subsequently sent to a monitor for viewing by the user. In the same way a surgeon would be relying on the monitor to view the surgical field within a patient, the user views the digital environment on the monitor while maneuvering the simulated laparoscope in connection with the training environment.

In accordance with various embodiments, reference to the training environment corresponds to an insert or grid that is configured to facilitate a tracking of the positions of the simulated laparoscope with respect to the training environment. As referred to hereafter, the “position” of the simulated laparoscope is characterized by its corresponding six degrees of freedom; in various embodiments, the “position” of the simulated laparoscope is identified as the distal end of the camera having x, y, z coordinates as well as rotational values associated with the simulated laparoscope (e.g., roll, pitch and yaw).

In various embodiments, the digital environment corresponds to a space defined by the training environment. The digital environment is displayed on the monitor for the user to view. In various embodiments the scope view generator is configured to incorporate computer-generated elements with the digital environment to provide various menu and camera navigation exercise functionalities. In various embodiments, the digital environment can include augmented reality embodiments where real-time image data (i.e. the image data obtained from the training environment) obtained from the simulated laparoscope is displayed on the monitor with computer-generated elements superimposed thereon.

In various embodiments, the computer-generated elements (generated by the scope view generator to be included with the digital environment) comprise elements such as buttons, targets, cursors, meters, and obstacles. In various embodiments, the computer-generated elements are used with the digital environment to provide menus as well as different camera navigation exercises for the purposes of teaching and training camera navigation-related skills.

In the various embodiments, all information for the computer-generated elements are stored in memory of the camera navigation system. For example, information about the computer-generated elements stored in memory include what camera navigation exercises they are used in, their specific locations within the digital environment when used for the camera navigation exercise, and/or characteristics (i.e. are the elements stationary, opaque, moving, shape). In various embodiments that are applicable to augmented reality, the computer-generated elements may be used to augment the image data captured by the simulated laparoscope to provide an augmented view that is displayed on the monitor. For example, various augmented embodiments provide the incorporation of various computer-generated elements, such as buttons and targets, superimposed on the image data that is displayed on the monitor. The respective placement of the computer-generated elements for the purposes of augmenting the image data would be stored in memory as well; identifying when the augmented elements are usable, where the augmented elements should be placed, and/or how do the augmented elements behave.

In various embodiments, memory used to store, for example, the information for the camera navigation exercises as well as the computer-generated elements used therein may be a memory storage device that is physically located at a same location as the scope view generator. In various embodiments, the memory may be or be included with or attached to a remote server or in the cloud (i.e. locations that are remote from the location of the scope view generator). In various embodiments, the scope view generator is configured to retrieve the appropriate information stored in memory to generate a menu and/or execute a camera navigation exercise or related operations on the camera navigation system.

In various embodiments, the camera navigation system is provided with at least one monitor. Through the use of the monitor, users of the camera navigation system are able to view the training environment as captured by a camera of the simulated laparoscope. In various embodiments, the training environment may be housed within a camera navigation box and/or surgical trainer. Thus, in accordance with various embodiments, a direct view of the training environment without the aid of a scope or camera is not possible or is limited/restricted. In various embodiments, the monitor is configured to provide a view to users to view a training environment in embodiments where a direct view is not generally possible. This viewing of the training environment (housed within a camera navigation and/or surgical trainer) through the use of a monitor simulates how surgeons would be required to view a surgical field within a body cavity of the patient during surgical procedures.

A highly skilled operation technique is typically required of surgical personnel, e.g., surgeons and is especially true for performing laparoscopic surgical procedures. In laparoscopic surgery, several small incisions are made in the abdomen for the insertion of trocars or small cylindrical tubes through which surgical instruments and a laparoscope are placed into the abdominal cavity. The laparoscope is used during surgery to illuminate the surgical field as well as capture and subsequently transmit a magnified image from inside the abdominal cavity of the patient's body to a monitor. The magnified image shown on the video monitor gives the surgeon a close-up view of the surgical field as well as nearby organs and tissues. The surgeon performs the laparoscopic surgical procedure by manipulating the surgical instruments placed through the trocars while watching the live video feed on the monitor transmitted via the laparoscope.

Because the surgeon does not observe the organs and tissues of the surgical field directly with the naked eye but rather relies on the live video feed on the monitor, challenges arise because the visual information of the three-dimensional space associated with the surgical field is instead obtained from a two-dimensional image on the monitor. The loss of information when presenting the three-dimensional environment via the two-dimensional image is substantial, for example, depth perception is reduced when viewing a two-dimensional image as a guide for manipulating the surgical instruments in three dimensions.

Furthermore, there are restrictions for the movements of the simulated laparoscope due to the anatomy of the patient. For example, because the trocars are inserted through small incisions and rest against the abdominal wall, the manipulation of instruments and the laparoscopes is restricted by the abdominal wall. Specifically, the abdominal wall creates a fulcrum effect on the instruments and laparoscopes used in the laparoscopic surgical procedure. The fulcrum effect defines a point of angulation that constrains the range of motion for the instruments and laparoscopes.

Furthermore, hand motions in one linear direction with the laparoscope can cause magnified tip motion in the opposite direction as seen on the monitor. Not only is the instrument and laparoscope motion viewed on the screen in the opposite direction, but also, the magnified tip motion is dependent on the fraction of the instrument and laparoscope length above the abdominal wall. The lever effect not only magnifies motion but also magnifies tool tip forces that are reflected based on the movement. Hence, the manipulation of surgical instruments as well as the laparoscope by the surgeon with a fulcrum is not intuitively obvious and thus require intentional learning that can be provided by the camera navigation system described herein.

In various embodiments, the surgical instruments and laparoscopes are placed through ports having seals. The seals induce a stick-slip friction. Stick-slip friction may arise from the reversal of tool directions when, for example, quickly changing from pulling to pushing on tissue. During slick-slip friction motion, rubber parts of the seals rub against the tool shaft causing friction or movement of the tool with the seal before the friction is overcome and the instrument slides relative to the seal. Stick-slip friction (also referred to as oil-canning) between the seal and instrument and/or laparoscope interface creates a non-linear force on the instrument and/or laparoscope that results in a jarred image on the live video feed shown on the monitor. The jarring resulting from the stick-slip friction can be distracting during a surgical procedure by a surgeon. Therefore, there is a need to practice the varying of the insertion depth of the laparoscope and surgical instruments to prevent or minimize the occurrence of the stick-slip friction.

Hand-eye coordination skills are also necessary and must be practiced. Hand-eye coordination skills during an actual surgical procedure correlate the hand motion with a tool tip motion within the body cavity of the patient as well as the tool tip motion shown via the live feed on the monitor. In laparoscopic surgery, tactile sensation through the tool is diminished because the surgeon cannot palpate the tissue directly with a hand. Because haptics is reduced and distorted, the surgeon must develop a set of core haptic skills that underlie proficient laparoscopic surgery. The acquisition of these skills is one of the main challenges in laparoscopic training and in accordance with various embodiments of the present invention (as described in further detail below), the present disclosure describes various embodiments aimed at providing a way for users to improve their camera navigation technique performances as well as other related surgical skills.

With various embodiments discussed in further detail below, a camera navigation system is provided having a digital environment that includes a variety of different computer-generated elements. The digital environment is influenced by the captured image data obtained by a simulated laparoscope from the training environment. To simulate the body cavity of a patient, a camera navigation box and/or surgical trainer is used. Correspondence between the simulated laparoscope's position with respect to the training environment and the display of the cursor within the digital environment is provided; in particular markers associated with the training environment are used to track the position of the simulated laparoscope and provide the correlation between the position and the cursor's position within the digital environment. The camera navigation system provides a view using computer-generated elements displayed on the monitor allowing users to simulate actual surgical methodologies of indirectly viewing the operating space and practicing as to where and how to move the laparoscope from one location to the next.

In various embodiments, the simulated laparoscope (having a camera sensor) is configured to capture images with respect to the training environment. The images captured by the simulated laparoscope are then used to generate the digital environment and other computer-generated elements (e.g., cursor) that can then be displayed on the monitor. In various embodiments, the simulated laparoscope has one or more sensors.

In various embodiments, the camera navigation system comprises the camera navigation box configured to simulate an operating space within the patient, a surgical trainer configured to simulate a patient's torso, a plurality of markers inside the camera navigation box, LED (light emitting diode) lighting, and/or a simulated laparoscope used to capture images of the markers from within the camera navigation box.

In various embodiments, the camera navigation system comprises a scope view generator that utilizes the information related to the position of the simulated laparoscope to generate and update the digital environment. In various embodiments, the simulated laparoscope is represented as a cursor within the digital environment. The cursor will be positioned within the digital environment based on the position of the simulated laparoscope denoted by the markers that were captured by the simulated laparoscope. The cursor can also be used as an activation mechanism that initiates and/or interacts with one or more computer-generated elements (e.g., buttons, targets) within the digital environment; the digital environment being displayed on the monitor for the user to view. A series of camera navigation exercises are provided to teach and practice camera navigation or surgical skills such as those that involve manipulation of a laparoscope within the body form of the patient.

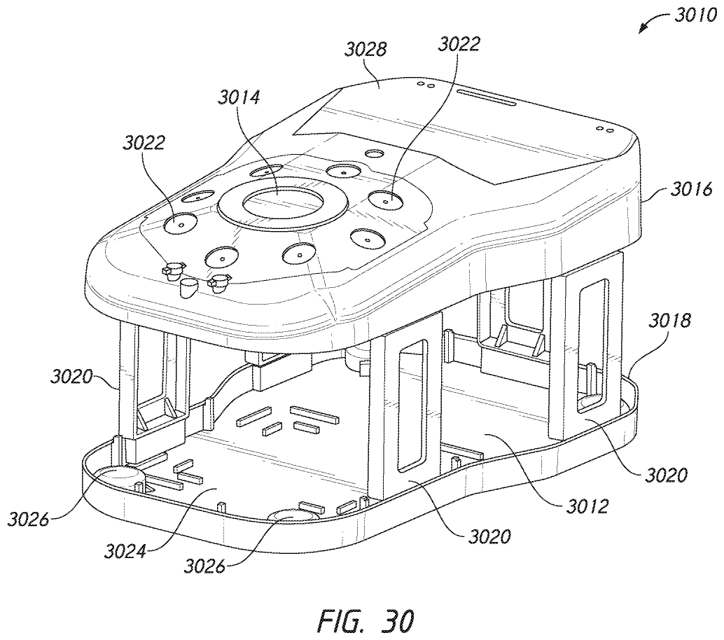

In accordance with various embodiments, the training environment may incorporate the use of a surgical trainer (3010). The surgical trainer (3010) is configured to simulate a torso of a patient. The training environment would be housed within the surgical trainer (3010), the surgical trainer (3010) being configured to obstruct a direct view to the training environment thereby necessitating reliance on the display of the digital environment on the monitor to inform how the user would need to maneuver the simulated laparoscope with respect to the training environment. In accordance with various embodiments, the surgical trainer (3010) has a top cover (3016) that is spaced apart from a base (3018) thereby defining the internal cavity (3024). Further details of the surgical trainer (3010) are provided below in connection with FIG. 30.

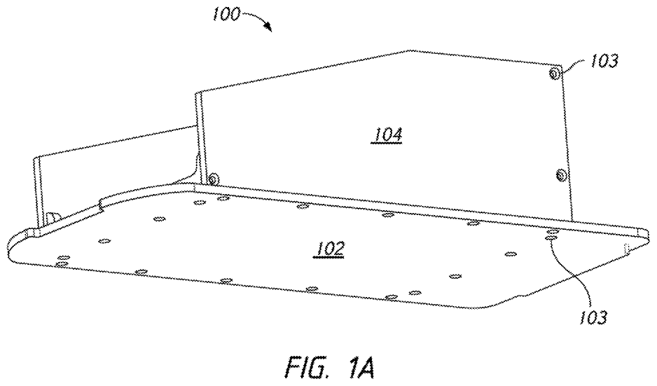

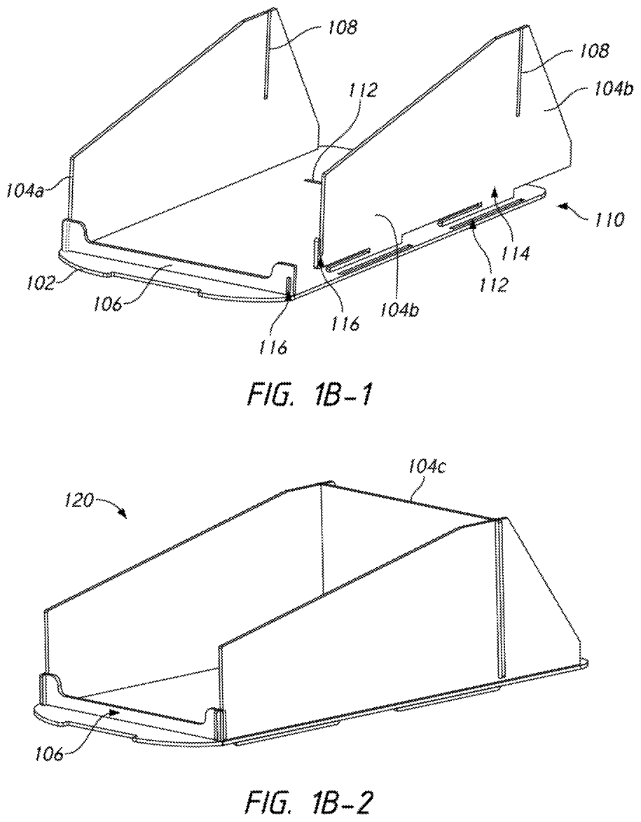

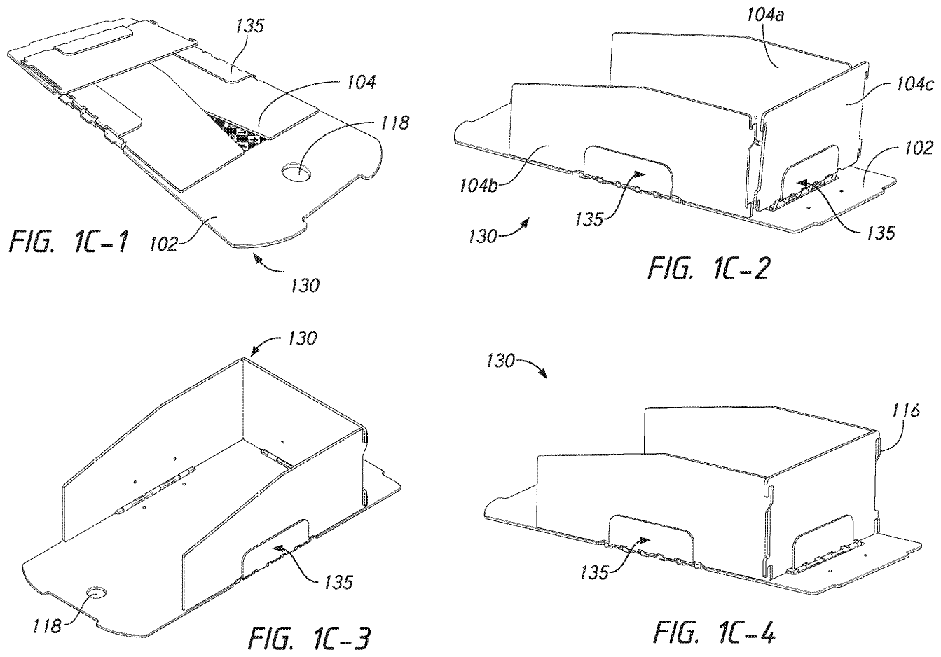

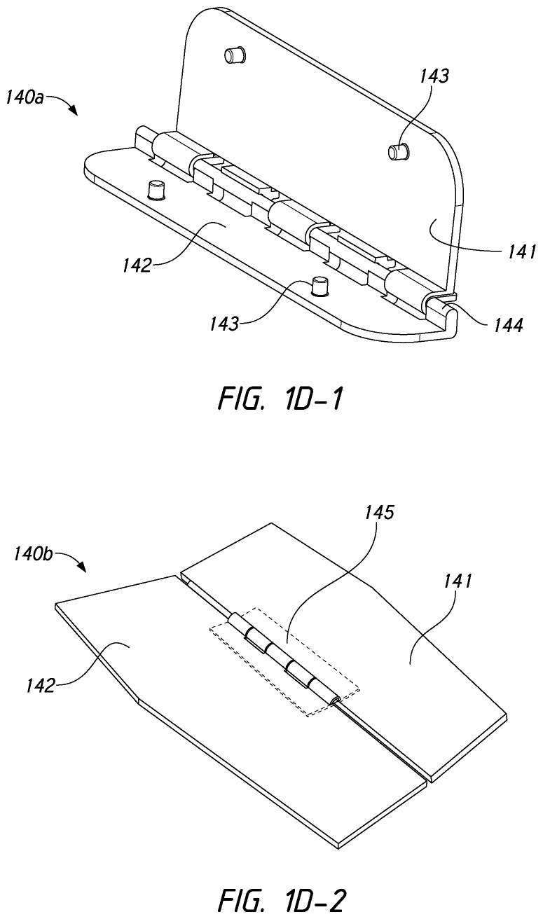

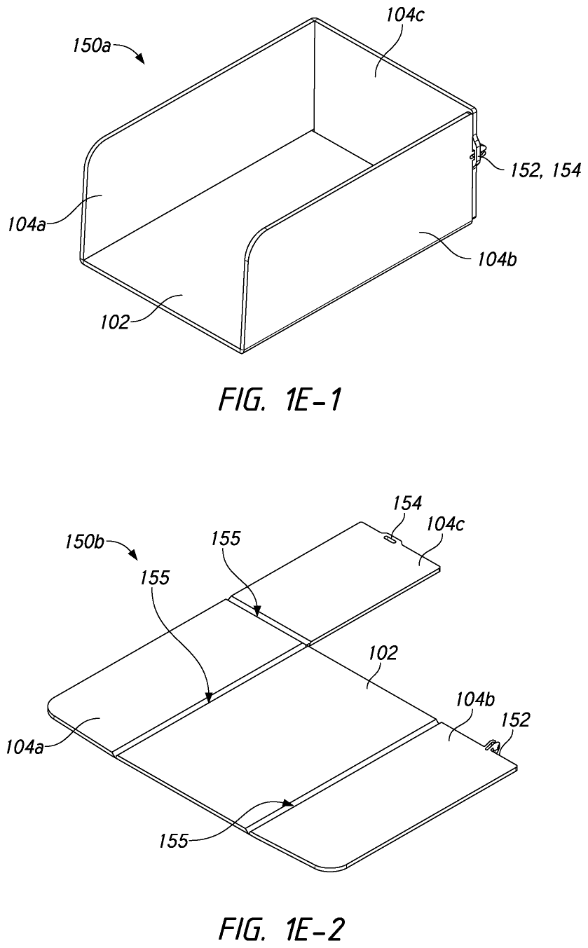

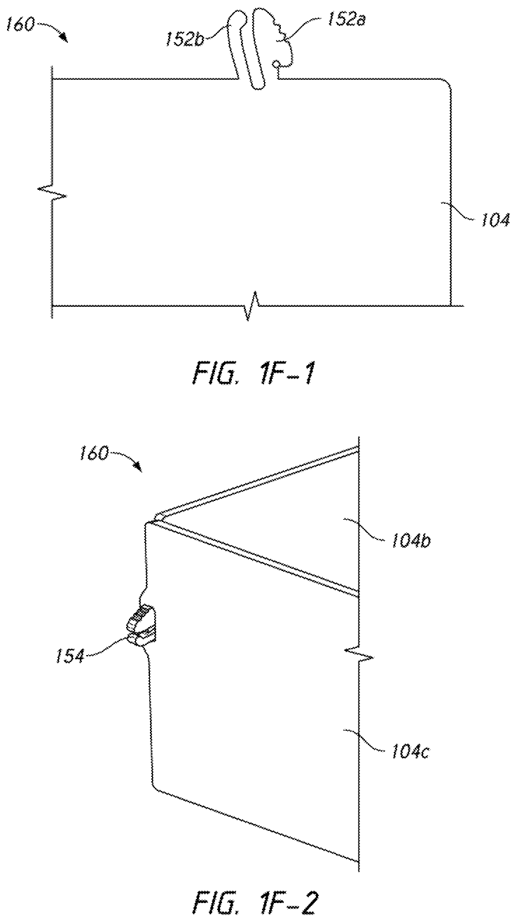

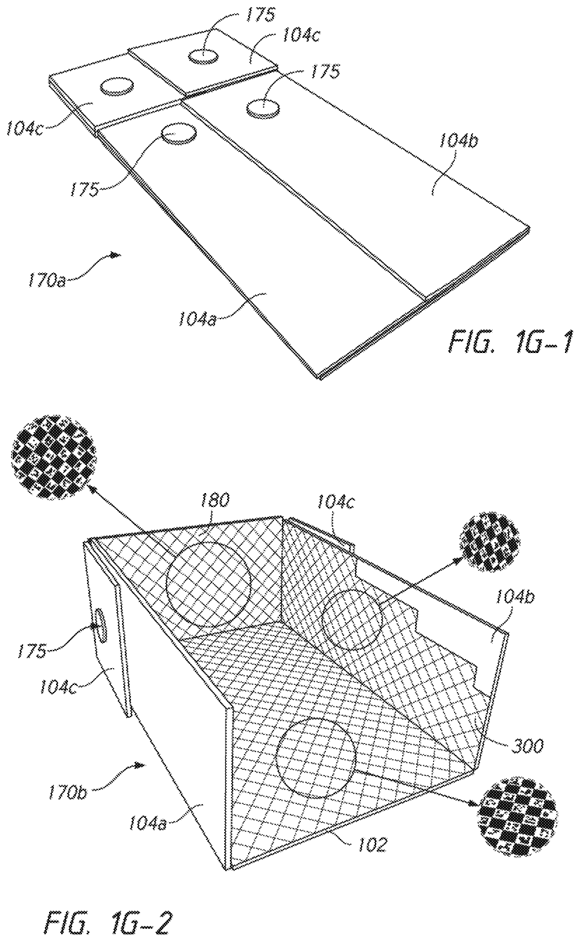

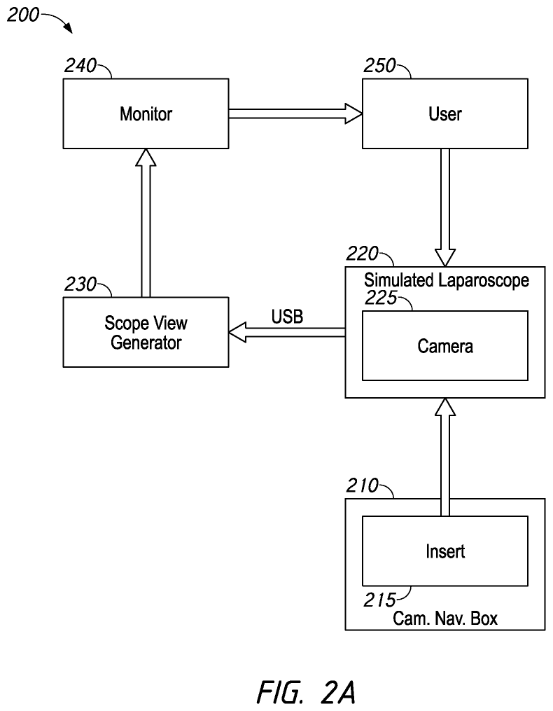

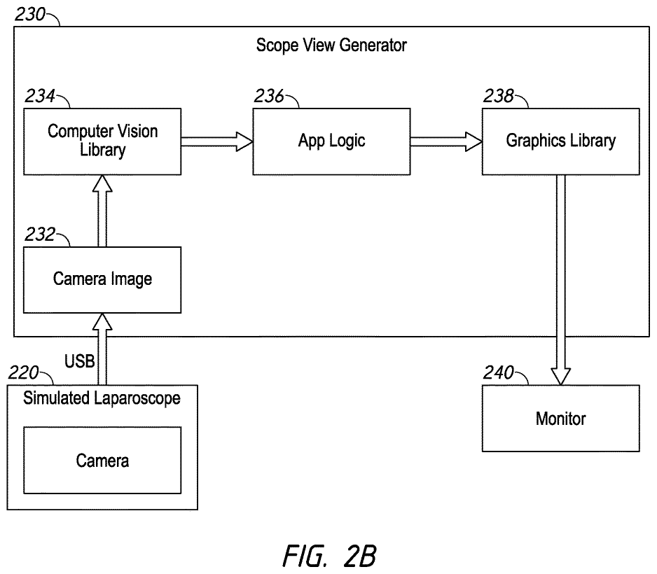

In accordance with various embodiments, the camera navigation system utilizes the training environment (i.e. the insert or grid having a plurality of unique markers) to track the position of the simulated laparoscope. The training environment can be housed within a camera navigation box (100); an example of which is shown in FIG. 1A. The insert or grid comprises a plurality of markers (e.g., QR codes) that are specially designed and arranged within the training environment to facilitate the identification of the positional information of a simulated laparoscope. As shown in FIGS. 1A-1G-2, various embodiments of the camera navigation box are provided. In the various embodiments, the camera navigation boxes are designed to house the insert or grid therein. For example, the insert or grid can be placed on the base (102) of the camera navigation box and/or placed on one or more side walls (104). Furthermore, the camera navigation boxes are capable of being placed within a surgical trainer, such as one that is illustrated in FIG. 30 to further simulate procedures inside a body cavity of a patient. Further details about the camera navigation boxes will be provided below.

As noted above, in accordance with various embodiments, the insert or grid can be configured to be placed within the camera navigation box (100) on the base and/or on the side walls. The digital environment (which is the digital simulation displayed on the monitor for the user to view) is configured to have a size or area that corresponds to the size or area covered by the insert or grid. Furthermore, there is a direct correspondence (e.g., 1-to-1) of the cursor's movements and the simulated laparoscope's position with respect to the training environment (e.g., insert or grid). In various embodiments, the position of the simulated laparoscope with respect to the training environment is represented as a cursor within the digital environment. So the user would be able to move the simulated laparoscope and expect the cursor to move within the digital environment in a similar manner.

FIG. 1A-F IG. 1G-2 illustrate various embodiments of a camera navigation box. The camera navigation box is designed to house the insert or grid. In an example embodiment, as shown in FIG. 1A, the camera navigation box (100) has a base (102) and one or more side walls (104). The base (102) and the one or more side walls (104) have apertures (103) used to attach the base (102) and side walls (104) to each other to form and maintain the shape of the camera navigation box (100). The base (102) and side walls (104) may be connected via screws, pins, or other connective structure that interface with the apertures (103) associated with the base (102) and side walls (104). In various embodiments, the camera navigation box (100) is designed to minimize an impact of outside lighting and reflections on the simulated laparoscope used therein.

Though many of the figures contained herein and the associated descriptions use and/or refer to a particular embodiment of the camera navigation box, it should be noted that the camera navigation box used in connection with the insert or grid can have any number of different shapes as needed to simulate the space where a simulated surgical procedure is being performed. In an embodiment the camera navigation box (100) may correspond to a rectangular box with a base and one or more side walls that define the rectangular box shape. In some embodiments, the camera navigation box (100) may have any other quadrilateral shape (e.g., square, trapezoid) defined by its base and use of one or more side walls. In some embodiments, the structure may have a different geometric shape (e.g., triangle, pentagon, circle).

The choice in the shape of the camera navigation box (100) may be useful in facilitating a foldability or collapsibility of the overall structure. For example, the ability for the camera navigation box (100) to fold or collapse would make transportation of the overall system easier since the overall system could be designed to take up less space and thus easier to transport.

As noted above, the camera navigation system is designed to allow for the tracking of the location of a simulated laparoscope using the insert or grid housed within the camera navigation box and displaying a corresponding location via a cursor within a digital environment seen on the monitor. Thus, for this tracking to work, the insert or grid needs to be arranged within the camera navigation box in an appropriate manner. In particular, the edges of the insert or grid positioned on the base would need to be aligned with the edges of the insert or grid positioned on one or more of the side walls such that the markers on each of the inserts or grids are aligned in the pre-determined pattern and order. The arrangement of the markers and their positions with respect to the training environment are stored in memory; therefore, deviations from what is expected can cause errors in the tracking that provides the positioning of the simulated laparoscope. Further details about the insert or grid and the associated markers are described in detail below.

In other embodiments, for example with angled laparoscope embodiments, a horizon may need to be defined with the insert or grid (300). The horizon can be customized based on the exercise being practiced upon. In various embodiments, the horizon can be established as the bottom of the image sensor for the angled laparoscope. With another embodiment, the camera or image sensor may be introduced into the camera navigation box and/or surgical trainer to view the training environment in various different directions, for example, inserting through a top portion or front portion to simulate different surgical procedures.

In various embodiments, the camera navigation box (100) is designed to house the insert or grid (300) can further include a top or ceiling portion. The top or ceiling portion can be used to further obscure the user's direct observation of the insert or grid (300) from the top of the camera navigation box. The top or ceiling portion may be useful in embodiments where the surgical trainer is not included.

In various embodiments, the (102) base of the camera navigation box (100) may be surrounded by one or more side walls (104). In various embodiments, the side walls (104) are each arranged perpendicular to the base (102). In various embodiments, the camera navigation box (100) may have the base (102) have one or more of its sides not include a side wall (104). The lack of a sidewall on one or more of the sides of the base (102) provides the camera navigation box (100) with an opening, for example, as seen in FIG. 1A, through which the simulated laparoscope can be inserted and access the training environment.

In various embodiments, the camera navigation box (100) is designed to be portable. The camera navigation box (100) with the insert or grid can be movable to different locations and used with different set ups. The portability of the camera navigation box (100), facilitated in part by the collapsibility/foldability of the camera navigation box (100), eases transportation of the camera navigation box (100) by making portions of the camera navigation system easier to store and/or move. By making the camera navigation box (100) be collapsible or foldable, this allows for portions of the camera navigation system to have a reduced or minimal overall footprint when being transported or stored away when not in use. In various embodiments, the camera navigation box (100) is also configured to be durable, easily manufacturable, and reusable.

In various embodiments, the insert or grid that is housed within the camera navigation box (100) comprises a non-reflective surface that prevents or minimizes reflections that may occur from lighting provided, for example, within the camera navigation box (100) or associated with the camera navigation system overall. In various embodiments, the non-reflective surface of the insert or grid is coated with or made of a minimally or non-reflective material, e.g., matte. In various embodiments, the training environment may be defined by the camera navigation box (100) instead of the insert or grid. In particular, instead of having the plurality of markers arranged on the insert or grid (e.g., printed on an adhesive paper adherable or otherwise attachable to one or more surfaces (i.e. the base and/or side walls) of the camera navigation box (100)), the plurality of markers can instead of placed directly onto the base and/or side walls of the camera navigation box (100).

In various embodiments, the insert or grid can be printed on sheets which are cut to the dimensions of the base (102) and/or side walls (104) of the camera navigation box (100) to ensure that the markers on the insert or grid can be properly aligned. In various embodiments, the inserts or grids used with the camera navigation box (100) are configured to be not only removable but also interchangeable with other different inserts or grids. This allows the same camera navigation box (100) to be compatible with different inserts or grids where each different insert or grid can be used for different situations (e.g., different laparoscopes, different camera navigation exercises, different amount of space). For example, the insert or grid can also have different shapes (e.g., square, rectangle, oval) based on the shape and size of the camera navigation box. In another example, the specifications or features associated with a particular type or brand of laparoscope may necessitate different sized markers so that the simulated laparoscope can properly capture the image data used to track the position of the laparoscope with respect to the training environment.

In various embodiments, the insert or grid has arranged thereon markers on the surface. These markers, for example QR codes, can be printed, etched, or otherwise applied onto the insert or grid. In various embodiments, the markers may not be provided on the insert or grid but rather printed, etched, or otherwise applied directly onto the base (102) and/or side walls (104) of the camera navigation box (100).

With reference back to FIG. 1A-F IG. 1G-2, the figures illustrate example embodiments of the camera navigation boxes that are designed to be foldable or collapsible. As mentioned above, the foldability or collapsibility of the camera navigation boxes provides an easier way of transporting at least a portion of the camera navigation system or storing portions of the camera navigation system when not in use.

In an example embodiment, FIG. 1B-1 illustrates two different points in assembly for of the camera navigation box (110) that is made of separate components (e.g., base (102), side walls (104), and a front portion (106)). These separate components (e.g., base (102), side walls (104), and a front portion (106)) may be manufactured and/or provided separately. However, these separate components are configured to be assembled and attached together. As seen in FIG. 1B-1, each of the components have slits (108, 112) that allow the components to slidingly connect to each other. In various embodiments, for example as illustrated in FIG. 1B-1, two side walls (104a and 104b) have slits (108) near the back of the camera navigation box (110). The slits (108) open from the top of the side walls (104a and 104b) and extend into the middle area of the side walls (104a and 104b). A third side wall component (104c) is provided to connect with the two side walls (104a and 104b). The slit for the third side wall (104c) starts from the bottom of the third side wall (104c) and extends up into the middle area. This allows the slit of the third side wall (104c) to interface with the slits (108) of the two side walls (104a and 104b) as ultimately shown in the arrangement for the camera navigation box (120). The third side wall component (104c) can be provided to maintain the upright arrangement of the two side walls (104a and 104b) with respect to the base (102).

In addition to the slits associated with the side walls (104a-104c) that facilitate attachment of the side walls to each other, the side walls (104a-104c) are also configured to attach to the base (102). To facilitate this connection, the base (102) has a number of slits (112) that are associated with the base (102). These slits (112) may be arranged around the perimeter of the base (102) and ultimately define the space of the training environment. In various embodiments, to connect the side walls (104a-104c) to the base, the side walls (104a-104c) may have various hook portions (114) at the bottom. These hook portion (114) are designed to be inserted through the slits (112) of the base (102) and then repositioned such that the side walls (104a-104c) cannot be pulled out without first realigning the hook portion (114) with the slits (112) of the base (102). The fully connected arrangement can be seen in FIG. 1B-2.

Furthermore, as illustrated in FIG. 1B-1 and FIG. 1B-2, the camera navigation box (110, 120) may also include a front portion (106). In various embodiments, the front portion (106) facilitates (with the other side walls (104a-104c) defining the perimeter of the training environment. In various embodiments, the front portion (106) is removable and allows for the insertion and removal of a insert or grid (discussed later below) used for camera navigation exercises. The front portion (106) of the camera navigation box (110, 120) illustrated in the figure may utilize complimentary slits (116) that would allow for the sliding engagement of the front portion (106) with the side walls (104a and 104b) much like how the third side wall (104c) is slidingly engaged with the side walls (104a and 104b). In some embodiments, adhesives can be used to attach and secure the connection between the base and the side walls as well as one of the side walls to other side walls.

When the components for the camera navigation box (110, 120) are disassembled (for example, in FIG. 1B-1) from each other, the camera navigation box (110, 120) has a minimal footprint. This allows for an easier means for transportation or storage for at least the camera navigation box portion of the camera navigation system.

With the various embodiments of the camera navigation box (illustrated in FIG. 1A-1G-2), the camera navigation box can be made of separate pieces or components. Therefore, the pieces or components can be standardized in manufacturing using a template for consistency. A benefit allows the manufacturing of the camera navigation box to be consistent and provides the ability to ensure that the dimensions of the camera navigation box are satisfactory to allow for the alignment of the markers on the insert or grid. In various embodiments, the pieces or components making up the portions of the camera navigation box (e.g., base, side walls) can be machined, laser cut, or die cut.

Furthermore, the manufacturing of the components separately and allowing the user the ability to assemble the camera navigation box allows camera navigation box (or at least its components) to be shipped with a smaller footprint. Once received, the user can assemble the components together using various different types of fasteners (e.g., screws, pins) that interface with the holes or apertures (103) and secures the base and sidewalls together. Furthermore, in various embodiments, the fasteners (e.g., screws, pins) can be removable to allow for disassembly of the camera navigation box. In various embodiments, the side walls can have dowel pins press fit into the base. Corresponding mating holes in each of the side walls facilitate connections between the base and the side walls.

In various embodiments, the components (e.g., base and side walls) of the camera navigation box may be attached and secured to each other via various hinges (135). The hinges (135) also allow for the components of the camera navigation box to be folded or collapsed into a flat formation during storage or transportation, as seen in the embodiment of the camera navigation box (130). In various embodiments, the hinges (135) are configured to be used to attach the side walls (104) with the base (102). The hinges (135) are generally arranged on the exterior of the camera navigation box to allow for a more planar or unobstructed interior surface for the camera navigation box.

In various embodiments, the side walls (such as the back side wall (104c)) may be attached to the adjacent sidewalls (104a and 104b) via complimentary hooks and slits (116) similar to how the front portion (106) was attached to the side walls (104a and 104b) illustrated in FIG. 1B-2.

With reference to FIG. 1C-1 and FIG. 1C-3, the base 102 may include a surgical trainer aperture 118 that would be used to secure the camera navigation box with a surgical trainer. In various embodiments, this surgical trainer aperture 118 would not be included. In this manner, the camera navigation box can be placed within the surgical trainer and be secured via other means (e.g., clips) or be left inside the surgical trainer unsecured. In various embodiments, when the surgical trainer aperture 118 is not in use for securing the camera navigation box to the surgical trainer, the surgical trainer aperture 118 can also be used by the user as a way for the user to handle the device. For example, the user can insert their finger into the surgical trainer aperture 118 to hold and carry the camera navigation box.

As discussed next, a variety of different types of hinges can be used to attach the side walls to the base. Example hinges (140a and 140b) are illustrated in FIG. 1D-1 and FIG. 1D-2. With respect to a first hinge (140a) illustrated in FIG. 1D-1, the first hinge may comprise of two hinge plates (141, 142) that are held together with a pin (144). This allows the pieces of the hinge (140a) to be manufactured separately and assembled later. In addition, the two hinge plates (141, 142) have structures (143) that extend up away from the surface of the hinge plates (141, 142). These structures (143) are usable to attach the hinge with corresponding apertures or holes on the base and/or side walls of the camera navigation box. With respect to a second hinge (140b) illustrated in FIG. 1D-2, a similar arrangement can be seen with two hinge plates (141, 142) that are connected together. However, instead of a pin (144), an overmolding (145) made of epoxy is used to attach the two hinge plates (141, 142). Such implementation provides a different (potentially cleaner) look compared to the use of the pin (144). However, the overmolding (145) may not always cure flat and can require additional processes to implement compared to utilizing the pin (144) described with the first hinge (140a).

In various embodiments, the hinges (135) may be made separately and/or provided separately from the base (102) and sidewalls (104) of the camera navigation box (100). This would require that the hinges (135) be attached to the base (102) and/or side walls (104), for example, by a user. In various embodiments, attachment between the hinge (135) and the base (102) and/or sidewalls (104) of the camera navigation box can be implemented using adhesive, overmolding, and/or hardware interface (e.g., screws). In various embodiments, the hinges (135) may be designed with the base (102) and/or sidewalls (104) as a single monolithic component. In various embodiments, the hinges (135) may be designed as separate plates (141, 142) that are connected with the base (102) and/or sidewalls (104). The separate plates (141, 142) making up the hinge can be subsequently connected (i.e. snap-fit) together during the assembly of the camera navigation box (100). Other types of hinges are possible so long as they allow for the components of the camera navigation box (100), such as the side walls (104), to move thereby allowing the camera navigation box (100) to fold and unfold.

In various embodiments, the hinges (135) may be designed to only open a pre-determined amount (e.g., 90 degrees). In various embodiments, the hinge (135) may be designed to lock into its current position once the pre-determined angle has been reached as the hinge is unfolding. An unlocking mechanism can be provided for the hinge (135) so that the hinge (135) can be re-folded.

With reference to FIG. 1D-1, the first hinge (140a) used to attach the base (102) with the side walls (104) can be a butt hinge. A butt hinge arrangement may be desired since it allows the detachment of the base (102) from the side walls (104) by removing the pin (144) that holds the two plates (141, 142) of the butt hinge together. This allows the base (102) and side walls (104) to become detached from each other without the need of disassembling the entire hinge (140a) from the base (102) and/or side walls (104). In various embodiments, other types of hinges are also considered including but not limited to continuous hinges, strap hinges, spring loaded hinges, leaf hinges, and countersunk mount hinges.

With reference to FIG. 1E-1 and FIG. 1E-2, additional embodiments of the camera navigation box are shown using “living hinges” (155). In particular, these embodiments illustrated in the figures provide an arrangement for the camera navigation box that is in an unfolded state (150b) as seen in FIG. 1E-2 and thus can be transported in a flat manner. FIG. 1E-1 illustrates an embodiment of the camera navigation box in its constructed form (150a).

As shown in FIG. 1E-2, the base (102) and sidewalls (104a-c) of the camera navigation box are attached to each other or otherwise made of one monolithic piece of material. As described herein, the base (102) and side walls (104a-c) of the camera navigation box are attached to each other via “living hinges” (155). In various embodiments, portions of the camera navigation box that are between the base (102) and the side walls (104a-c) have been modified to form beveled grooves (155). In various embodiments, the base (102) and the side walls (104a-c) can be separate components. A separate material can then be used to connect the base (102) and the side walls (104a-c) together to act as the “living hinge” (155). The separate material can then be configured to have a similar form as the beveled groove (155).

In various embodiments, the beveled grooves (155) are located at the corner junctions between the base (102) and/or sidewalls (104a-c). The beveled grooves (155) are what allow the base (102) and sidewalls (104a-c) the ability to flex and bend. In particular, the beveled grooves (155) would allow for the arrangement of the camera navigation box while it is in use (150a) to then swap to an unfolded state (150b) that allows the transportation or storage of the camera navigation box that is essentially flat.

In various embodiments, a hook 152 and corresponding slot 154 portion can be provided between the side walls (104a-c) that would facilitate the connections between adjacent side walls (104a-c). The use of the hook 152 and slot 154 would facilitate maintaining the shape of the camera navigation box, for example, as a box or rectangular arrangement.

In various embodiments, the flexing and bending provided by the beveled grooves (155) can be limited (e.g., 90 degrees). In various embodiments, the physical limitations of the separate material used to form the living hinge (155) can also affect the flexibility of the “living hinge.” In various embodiments, additional braces or structures can be added with the beveled grooves (155) to ensure that the flexing and bending can also be limited, for example, to no larger than 90 degrees. In various embodiments, the side walls can utilize structures like the hook 152 and slot 154 to maintain the upright arrangement for the living hinge (155) thereby controlling an extent the “living hinge” (155) can flex and bend.

In various embodiments, the “living hinge” (155) between the base (102) and the sidewalls (104a-c) can include materials that are elastic enough to bend without shattering such as polypropylene. In various embodiments, the insert or grid could be arranged on the base (102) and/or side walls (104a-c) and additional portions associated with the insert or grid be used as a “living hinge” to not only attach the base (12) with the side walls (104a-c) but also provide the folding and unfolding capabilities of the camera navigation box. The insert or grid could be suitable since the insert or grid can be made of an elastic material (e.g., vinyl) that would provide the necessary flexibility. The insert or grid can then be designed to allow the side walls (104a-c) to be positioned perpendicular to the base (102) and the markers on the insert or grid can be designed to be aligned once the sidewalls are in the appropriate arrangement. In various embodiments, a “gap” or spacing between the visible portions of the insert or grid that display the markers and portion of the insert or grid is provided that would function as the “living hinge” (155) that would be obscured when the base (102) and one or more of the side walls (104a-c) are folded into a desired arrangement to form the camera navigation box.

Referring back to FIG. 1E-1, the hook (152) and slot (154) provides the ability to attach adjacent side walls (104a-c). In various embodiments, other structures such as magnets (described below), Velcro, and complementary snaps can also be used. In any case, the hook (152) and slot (154) are shown up closer in FIG. 1F-1. In particular, in various embodiments, the hook (152) is made of two parts: a first portion with teeth (152a) and a second portion that expands (152b). When interfacing with the slot (154), the first portion with teeth (152a) would have the teeth come into contact with the top portion of the slot (154). With the slot (154) in one of the grooves of the first portion with teeth (152a), this would prevent any backwards motion while the hook (152) is engaged. The second expanded portion (152b) is biased in an outward direction which would provide pressure onto the teeth portion (152a) to engage with the slot (154). To insert the hook (152), both the teeth and expanded portion (152a and 152b) of the hook (152) are depressed and the hook (152) inserted into the slot (154). The expanded nature of the second portion (152b) prevents removal of the hook (152) from the slot (154). The insertion of the hook (152) into the slot (154) can be seen in FIG. 1F-2. To remove the hook (152) from the slot (154), the two portions (152a and 152b) of the hook (152) are depressed again prior to removal from the slot (154).

The attachment of the side walls (104a-c) help defines the space for the training environment by maintaining a perimeter corresponding with the side walls (104a-c). In various embodiments, the side walls (104a-c) also provide additional surfaces for the training environment to be arranged upon providing more of a three-dimensional space that can be tracked. Furthermore, the attachment also helps maintain the arrangement of the side walls (104a-c) to be perpendicular with the base (102). In various embodiments, connectors and/or related attachments to facilitate, hold and/or lock the side walls (104) together may be manufactured separately from the base and/or side walls and subsequently assembled. In various embodiments, such as those described throughout, the attachments may be embedded or at least manufactured with the base (102) and/or side walls (104).

FIG. 1G-1 and FIG. 1G-2 illustrate additional embodiments of the camera navigation box. In particular, FIG. 1G-2 illustrates an embodiment that shows how the camera navigation box is assembled using magnets (175) that are used to attach the side walls (104a-c) together and to maintain the perpendicular arrangement of the side walls (104a-c) to the base (102). In various embodiments, the magnets (175) may be embedded within the side walls (104a-c), secured using adhesives, or some combination of both. In contrast, FIG. 1G-1 illustrates an embodiment of the camera navigation box while in a folded/collapsed state where the side walls (104a-c) are folded to the base (102). In the folded/collapsed state, the camera navigation box is easier to transport and store away when not in use with the rest of the camera navigation system.

In various embodiments, two different arrangements of an example camera navigation box are shown. In various embodiments, a first arrangement provides the camera navigation box in a folded/flat arrangement (170a) and a second arrangement provides the camera navigation box unfolded(170b). In various embodiments, two side walls (104a-104b) may have magnets or have magnetic materials that are of opposite polarity with the magnets (175) associated with a third side wall (104c). In various embodiments, two side walls (104a-104b) may be made of a material that can be attracted by the magnet (175) of a third side wall (104c).

As shown, an insert or grid (300) is placed inside the camera navigation box. In various embodiments, an insert or grid (300) is placed on the base (102) of the camera navigation box. In various embodiments, an insert or grid (300) is placed on one or more of the side walls (104a-c). In various embodiments, an insert or grid (300) may cover the entirety of the base (102) and/or side walls (104a-c) or at least a portion thereof. In any case, the area defined by the insert or grid (300) corresponds to the training environment and in turn the space being represented via the digital environment displayed on the monitor.

As illustrated in FIGS. 1A-1G-2 various embodiments of for the camera navigation box or portions thereof are foldable or collapsible. In various embodiments, the camera navigation box is configured in that the plurality of markers on the insert or grid (300) can be aligned appropriately across each of the internal surfaces (e.g., base and/or one or more of the side walls) of the camera navigation box. In particular, the plurality of markers on the insert or grid (300) are arranged to be clear and uninterrupted. In various embodiments, the plurality of markers follows a pattern that is consistent on the different internal surfaces of the camera navigation box. Furthermore, transitions between one surface (e.g., base (102)) to a different surface (e.g., a side wall (104(a-c)) are arranged to not interfere with the pattern provided by the plurality of markers on the insert or grid (108). An example of a specific arrangement of markers on the insert or grid (300) on multiple surfaces can be seen, for example, in FIG. 1G-2, where the camera navigation box in the unfolded state (170b) has the plurality of markers that can be seen on the base (102) and seamlessly transitioning to other surfaces such as the other side walls (104a-c).

The alignment of the markers associated with the insert or grid (300) ensures that the camera navigation system will be able to accurately process groups of adjacent markers to identify a position of the simulated laparoscope with respect to the insert or grid (300). In various embodiments, a minimum number of four markers would need to be acquired by the simulated laparoscope. The four markers would allow for various types of information related to the position of the simulated laparoscope relative to the training environment to be calculated/identified by the camera navigation system. Using any less than four markers may allow for the identification of some of the information related to the position of the simulated laparoscope relative to the training environment but may not be able to confirm the exact or all of the related information accurately. Using more than four markers may require the camera navigation system to perform more processing which may take time which may result in slower updating of the digital environment. In various embodiments, the number of markers needed may be based on the size of the markers, the type of simulated laparoscope (e.g., its field of view), and/or the type of markers used. A combination of markers are able to uniquely correspond to a particular location within the training environment (i.e. insert or grid). In various embodiments, computer vision is used by the camera navigation system to identify and determine which individual markers are present in the image data captured by the simulated laparoscope in order to pinpoint where in the training environment the simulated laparoscope is being pointed at/towards. Once the location of the marker(s) are known, the camera navigation system is able to estimate the positional information of the simulated laparoscope with respect to the training environment (via a PnP process in accordance with various embodiments); the position, in various embodiments being characterized by six degrees of freedom for the simulated laparoscope (e.g., x, y, z and roll, pitch, yaw). With the positional information obtained, the camera navigation system knows how the simulated laparoscope is currently being held with respect to the training environment.

If the alignment for some of the markers is off or outside an acceptable degree of error (such as the markers on the base being misaligned with the markers on one of the side walls), the camera navigation system may not recognize the combination of markers and thus may not be able to identify where in the training environment the simulated laparoscope is pointing towards. An error condition may then be raised which may prevent the camera navigation system in pinpointing the current position of the simulated laparoscope. Furthermore, the error condition could interfere or introduce errors with the digital environment that is being generated for the user to view on the monitor.

In various embodiments, if the simulated laparoscope is pointed towards a space where no markers are located (e.g., a side wall with no markers), a similar error may be raised as the camera navigation system may be unable to identify the current position of the simulated laparoscope. However, what may be displayed on the monitor may be different. For example, the digital environment may provide a notification that the simulated laparoscope is outside the training environment and provide an indication to the user to re-maneuver the simulated laparoscope to point towards the training environment. In another embodiment, the monitor may instead provide a real-time image of what information is currently being captured by the camera sensor of the simulated laparoscope (e.g., an open area or an empty side wall of a camera navigation box that doesn't have any markers thereon). As such, the camera navigation system would provide a clear indication that the user needs to maneuver the simulated laparoscope towards the training environment.

In various embodiments, errors may include the inability to maintain a stable digital environment (inclusive of the computer-generated elements incorporated therein). Such a scenario would inhibit the user's interactions with the camera navigation system. For example, the view of the digital environment shown on the monitor may shake back and forth frame to frame as the camera navigation system is trying to update the digital environment based on the miscalculations that the simulated laparoscope is located at two different locations at the same time due to the misaligned markers. Alternatively, the view of the digital environment may be unchanged (or frozen) despite movements of the simulated laparoscope as no updates have been received by the camera navigation system. As noted above, notifications can be provided to direct the user to move the simulated laparoscope in a specific direction in order to be within the pre-determined area to be trackable again (i.e. directing the simulated laparoscope towards one of the markers in the training environment).

In accordance with various embodiments, the camera navigation system can be run locally, remotely, or partially locally and partially remotely. In various embodiments, remote applications refer to implementation of at least a part of the camera navigation system such as the scope view generator portion on a cloud-based server or a remote server whereby the remote portion is physically remote from at least the training environment and the user. In various embodiments, data associated with the camera navigation exercises and/or the plurality of markers may be stored in the same manner: locally, remotely, or partially locally and partially remotely.

In accordance with various embodiments, the camera navigation system provides different surgical exercises directed at practicing surgical skills corresponding to using a laparoscope, endoscope, or the like during a surgical procedure in connection with the training environment (e.g., the insert or grid). In various embodiments, the training environment for the camera navigation system is compatible with different laparoscopes (e.g., being made from third parties or having different features such as being zero-degree or angled (e.g., 30°)).

In accordance with various embodiments, example camera navigation exercises provided by the camera navigations system for use with a zero-degree laparoscope comprises a follow exercise, a track exercise, and/or a framing exercise. The follow exercise requires maneuvering the cursor within the digital environment to follow a path as displayed on the monitor. The track exercise requires maneuvering the cursor within the digital environment to follow a moving target as displayed on the monitor. The framing exercise requires maneuvering the cursor to overlap one or more targets within the training environment as displayed on the monitor. The maneuvering of the cursor within the digital environment is carried out by using similar motions with the simulated laparoscope with respect to the training environment.

In various embodiments, the same camera navigation exercises are usable in connection with an angled laparoscope. However, in various embodiments, additional exercises are provided for a simulated laparoscope having an angled feature. Example exercises provided for the angled laparoscope comprises tube targeting and/or star pursuit. The tube targeting exercise requires the maneuvering of the “perspective” of the cursor to center about a target that is planar with a viewing surface within the digital environment and a tube which extends perpendicular from the target. The star pursuit exercise requires maneuvering the “perspective” of the cursor within the digital environment to track and follow a position of the star as it is moved from one location to another within digital environment. Again, the maneuvering within the digital environment is carried out by the maneuvering of the simulated angled scope or portions thereof with respect to the training environment. Further details of each of these camera navigation exercises will be provided below.

One reason that there is a difference in the type of camera navigation exercises available between the zero-degree and angled laparoscope is due to the physical properties of the respective laparoscopes. With the zero-degree laparoscope, the camera/image sensor is aligned with the longitudinal axis of the simulated laparoscope. However, the camera/image sensor is not aligned with the longitudinal axis of the simulated laparoscope in the angled embodiment but rather is presented at an angle (e.g., 30 degrees). This provides additional complexity regarding how the camera/image sensor can be rotated and manipulated with respect to the training environment.

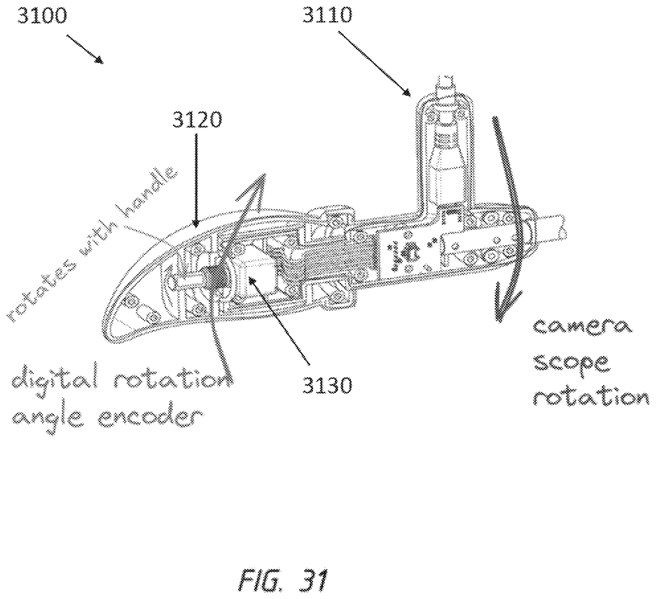

With reference to FIG. 31, an example figure is provided which illustrates portions of an angled laparoscope (3100) in accordance with various embodiments. In particular, the angled laparoscope (3100) can be rotated via two different points of manipulation: a first point (3120) is located at the proximal end of the angled laparoscope. Rotating the angled laparoscope (3100) using the first point of manipulation (3120) has the effect of rotating the image being captured by the angled laparoscope much like if the user physically rotated the zero-degree laparoscope. Thus, a 180-degree rotation using this first point (3120) would result in the captured image being upside-down.

With respect to the second point of manipulation (3110), this point is located between the distal and proximal ends of the angled laparoscope. The second point of manipulation (3110) is configured to physically rotate the camera/image sensor. The physical rotation of the camera/image sensor is used in order to change the direction the angled portion of the angled laparoscope, e.g., a distal end of the angled laparoscope, is directed towards. The physical rotation of the camera/image sensor (which in turn changes where the angled portion is facing towards) provides the ability for the angled laparoscope to view different areas of the training environment even though the position (other than its rotation) of a distal end of the angled laparoscope has not changed. This motion allows the angled laparoscope to “look around” objects. Such changes in the view are not possible or limited and, in various embodiments, are not provided in some zero-degree laparoscope

With the added complexity in what rotations are present with the angled laparoscope, a rotational sensor/encoder (3130) that measures the amount of rotation using one or both of the points of manipulation (3110, 3120) is provided. In various embodiments, such as the embodiment shown in the figure, the rotational sensor (3130) can be housed within the handle of the angled laparoscope.

In the embodiments associated with the zero-degree laparoscope, the scope view generator is able to calculate the 6 degrees of freedom based on the image data of the markers of the training environment. The same calculations can also be performed for the angled laparoscope. In various embodiments, an angled laparoscope or a scope with a rotational sensor, the camera navigation system is arranged to account for measurements made by the rotational sensor. In various embodiments, for example, the roll value for such a laparoscope is further modified by the data obtained via the rotational sensor (3130).

In various embodiments the camera navigation system is configured to identify which set of exercises should be loaded up and provided to the user upon detection of the simulated laparoscope being connected with the camera navigation system. In various embodiments, the camera navigation system may be configured to allow an angled laparoscope to access all the available camera navigation exercises. However, due to the physical limitations of the zero-degree laparoscope (i.e. not being able to “look” around corners), the zero-degree laparoscope is prevented from accessing exercises that are designated as skills related to applications for the simulated angled laparoscope. In embodiments where the simulated angled laparoscope is used, since there may be differences in how the image data is captured and used between a zero and angled laparoscope, the computer navigation system is configured to process and calibrate data obtained from the image data so that the display and update of the digital environment can be processed and provided uniformly.

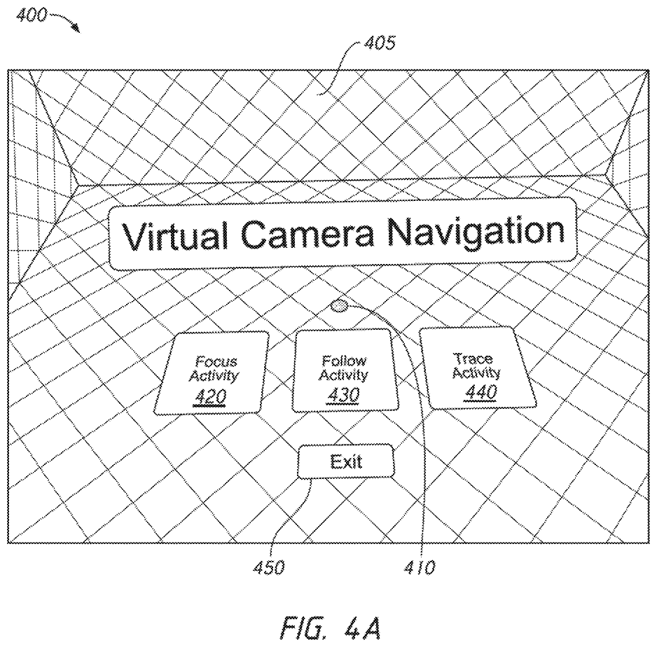

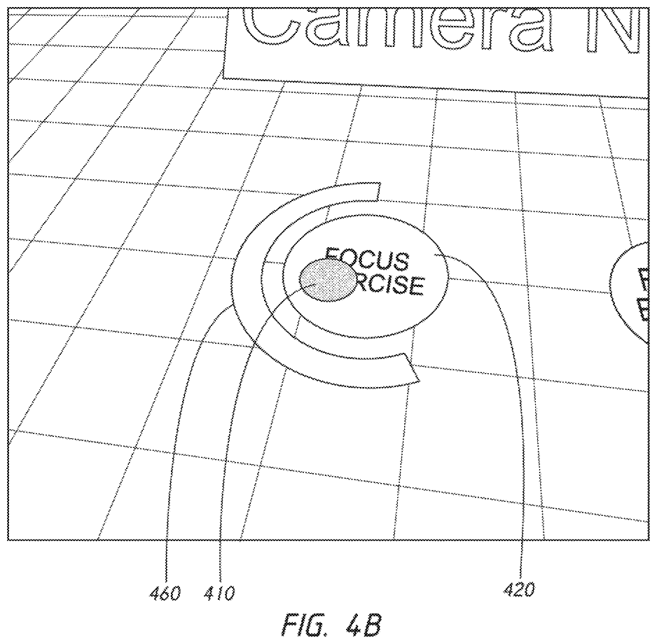

With reference to FIGS. 2A-2C, example embodiments for the camera navigation system (200) are illustrated. With reference to FIG. 2A, the camera navigation system (200) comprises a camera navigation box (210), a plurality of markers (e.g., markers) on an insert or grid (215), a scope view generator (230), a monitor (240), and/or a simulated laparoscope (220) (e.g., a simulated 0° or angled laparoscope) with a corresponding camera (225). In various embodiments, the camera navigation system (200) is configured to identify the types of exercises which should be presented to the user (250) via the monitor (240) and how the captured image data from the simulated laparoscope (220) is utilized based on the type of laparoscope (e.g., zero-degree or angled) connected thereto. In various embodiments, the camera navigation system (200) identifies the type of simulated laparoscope (220) connected and subsequently retrieves and displays a set of camera navigation exercises corresponding to the connected simulated laparoscope via a menu (as seen, for example, in FIG. 4A). In various embodiments, the user (250) may be able to submit or provide to the camera navigation system (200) (e.g., a selection from a user interface) the type of simulated laparoscope (220) being used which results in the retrieval, selection, and/or displaying of the appropriate exercises. In various embodiments, the identification is performed automatically as the camera navigation system would identify the connected simulated laparoscope and retrieve the corresponding information (e.g., camera navigation exercises, calibrations).

As discussed above, the camera navigation system (200) tracks the position of the simulated laparoscope via use of the markers on the insert or grid, which can be arranged on one or more of the planar internal surfaces within the camera navigation box (210). In various embodiments, the insert or grid (215) can be removed and the plurality of markers can be arranged directly (e.g., printed) on the internal surfaces of the camera navigation box (210). In various embodiments, the insert or grid (215) are placed on or otherwise attached (e.g., via adhesives) to the internal surfaces of the camera navigation box (210). In various embodiments, the insert or grid (215) may be interchangeable or replaced with other inserts or grids which may have a different arrangement of markers used for different camera navigation exercises.

In various embodiments, a simulated laparoscope (220) comprises a camera (225) (also referred herein as an image or camera sensor) that is used to capture image data of a subset of markers from the plurality of markers associated with the insert or grid (215). In various embodiments, the scope view generator (230) is provided; specifically configured to estimate a current position or scope view/perspective of the simulated laparoscope and generate a representation of the current position or scope view within the digital environment. The scope view generator (230) calculates the position-related information for the simulated laparoscope from the captured image data; in various embodiments, specifically by identifying the combination of markers and confirming the marker's location with respect to the training environment. In various embodiments, the scope view generator is configured to perform numerous calculations to further extract positional data about the simulated laparoscope (e.g., PnP process). In particular, from the image data obtained, the scope view generator is capable of obtaining information described as the 6 degrees of freedom for the simulated laparoscope. This information is able to “recreate” or at least define how the simulated laparoscope is being held at or positioned within the three-dimensional space defined by the training environment.

In various embodiments, the scope view generator is configured to generate and update the digital environment simulating the three-dimensional space corresponding to the training environment. In various embodiments, the scope view generator is configured to generate menus and camera navigation exercises for the identified simulated laparoscope that is connected and being used with the camera navigation system. In various embodiments, a current position for the simulated laparoscope with respect to the training environment has a corresponding representation within the digital environment. In various embodiments, the representation (e.g., circle) is used as a designator or cursor within the digital environment. Thus movements relative to the training environment with the simulated laparoscope will be shown as movements with the cursor in a similar manner.

The digital environment and the computer-generated elements implemented therein (such as the cursor and any targets or obstacles associated with the camera navigation exercises or menus being displayed) are provided to the monitor for the user to view. With the present application, user interaction (via use of the cursor) with the computer-generated elements (e.g. buttons, target) allow for simulation of camera navigation exercises that users are able to practice outside of the operating room by maneuvering the simulated laparoscope with respect to the training environment. In accordance with various embodiments, the camera navigation system is configured to provide opportunities for users to train outside of the operating room.

FIG. 2A-F IG. 2C illustrate various embodiments of a camera navigation system. With reference to FIG. 2A, the figure illustrates an embodiment of the camera navigation system. In particular, the figure illustrates a data flow regarding how the image data being captured via the simulated laparoscope (220) when interacting with a training environment (215) is processed via the scope view generator (230) and subsequently generate and display the digital environment on the monitor (240).

In various embodiments, the image data captured by the simulated laparoscope (220) via its camera sensor (225) is transmitted to a scope view generator (230). The scope view generator (230) is configured to process the image data to identify a position of the simulated laparoscope (220) with respect to the training environment (215) and/or generate and update the digital environment (which includes various computer-generated elements) incorporated therein that will be displayed on the monitor (240). The digital environment is generated and/or updated to provide menus and camera navigation exercises. In various embodiments, computer-generated elements are incorporated into the digital environment such as, background images, text, cursors, buttons, and/or meters can provide feedback about a current performance. The information or data about the computer-generated elements, in various embodiments, are all stored in memory and retrievable by the scope view generator (230).

In various embodiments, such as those shown in FIG. 2A, the camera navigations system (200) may be set up at a physical location (e.g., school, hospital) and operate locally at that physical location. In various other embodiments, such as those illustrated in FIG. 2C, portions of the camera navigation system may be set up and operated remotely (e.g. via the cloud, remote networks and/or remote systems).

In various embodiments, a user refers to an individual who uses or otherwise interacts with the camera navigation system in connection with practicing and/or training with its various camera navigation exercises. In various embodiments, the user would be manipulating the simulated laparoscope around the training environment and capturing images of the markers on the insert or grid. The user can view the corresponding digital environment and computer-generated elements (such as the cursor corresponding to the simulated laparoscope's position with respect to the training environment) on the monitor. By practicing different camera navigation exercises provided by the camera navigation system, increased proficiency in camera navigation can be realized that would be helpful for surgical procedures that are laparoscopic in nature, that have limited visibility and/or are involved in confined spaces.

In various embodiments (for example, as illustrated in FIG. 2A), the camera navigation box (210) for the camera navigation system (200) corresponds to an enclosed or partially enclosed space (e.g., a confined surgical operating space such as within the pelvis). In various embodiments, the camera navigation system (200) can also further simulate enclosed spaces via use of a surgical trainer to simulate the torso of a patient (of which details of one such embodiment will be provided below in connection with FIG. 30).

In various embodiments, the insert or grid is provided to facilitate tracking of the simulated laparoscope's position. A representation of the space defined by the insert or grid is the digital environment displayed on the monitor. The monitor shows a representation of the laparoscope's position with respect to the training environment as a cursor within the digital environment. The camera navigation system is configured to regularly obtain image data (e.g., 60 times per second) from the simulated laparoscope to allow the camera navigation system to continually update the cursor location within the digital environment.

In various embodiments, the camera navigation system is configured to run the camera navigation exercises in real time. Every time the display of the digital environment is updated on the monitor, the updated position of the cursor (corresponding to the current position of the simulated laparoscope with respect to the training environment) has already been computed and displayed. If for some reason the camera navigation system is unable to complete the necessary processing to identify and provide the updated positional information of the simulated laparoscope to be implemented into the digital environment, that specific frame updating the laparoscope's position may be skipped, the current frame on display is kept, and the next image data is retrieved and processed.

In various embodiments, the digital environment is configured to provide a perspective of the simulated laparoscope with respect to the training environment. This is to simulate the real-time scenario of a surgeon viewing the surgical field on the monitor while the surgical laparoscope is being maneuvered within the patient.

The digital environment being displayed on the monitor is based on the image data being captured by the simulated laparoscope. The image data is processed by the camera navigation system, and the scope view generator is configured to generate and update the digital environment accordingly. Furthermore, any subsequent calculations involved with quantifying the user's performance during performance of a camera navigation, for example, in connection with tracking targets, following paths, viewing distance, and collisions are performed by the camera navigation system and/or updated directly to the digital environment. In various embodiments, no reference is made to the simulated laparoscope or the physical set up associated with the training environment at least until the next update to the digital environment is needed. For example, a collision calculation regarding a user's perspective associated with the position of the simulated laparoscope between a target and a tube that encloses the target is performed based on information associated with the camera navigation exercise within the digital environment and the computer-generated elements included therein. The camera navigation exercise would have data about where the targets are located, where the tubes are located, and the current position and perspective of the cursor within the digital environment. The calculations related to the user's current perspective associated with the simulated laparoscope's position in relation to the targets and tubes are performed within the digital environment and the stored data related to the computer-generated elements (e.g., targets and tubes). This calculation could be represented, presented, and/or determined by generating a line between the cursor and the target and detecting whether any computer-generated elements (e.g., tube) has positional information that intersect that line.

In another embodiment, the camera navigation system may utilize a star pursuit exercise (discussed in detail in the application) in connection with an angled laparoscope. Once the position and perspective of the angled laparoscope is known within the digital environment, that location of the angled laparoscope can be compared with data that is stored with the star pursuit exercise that corresponds to, for example, a current location of the star, how the star moves from one location to another, and/or the location/arrangement of the obstacles within the digital environment. Calculations are performed within the camera navigation exercise, for example by comparing coordinates or other means of comparing position within the digital environment between the cursor and the star, to determine whether the star is at least being followed or otherwise properly viewed by the simulated angled laparoscope.

In various embodiments, similar functions are also present for the zero-degree laparoscope and their associated camera navigation exercises. For each of the camera navigation exercises (e.g., follow, track, framing), the camera navigation system has information regarding how the camera navigation exercises are run, e.g., operational steps, states, and/or conditions, and how the computer-generated elements (e.g., objects, tracks, targets) are defined (e.g., shape), located (e.g., x, y, z, coordinates), and/or behaves (e.g., movable, static). Once the camera navigation system identifies the current position of the simulated laparoscope with respect to the training environment, that information is converted (via the PnP process to achieve the 6 degrees of freedom for the zero-degree laparoscope), and the digital environment updated accordingly (e.g., placement of the cursor representing the simulated laparoscope's position relative to the training environment). The current position of the simulated laparoscope can be characterized with respect to the training environment as a combination of (x, y, z) coordinates which would correspond to coordinates within the digital environment where the cursor would be located. By using the coordinates of the cursor, the camera navigation system can compare the current position of the cursor with stored data about one or more of the computer-generated elements within the digital environment (e.g., tracks, targets, objects). Calculations can be performed between the coordinates of the cursor and the stored information (e.g., position-related information about each of the computer-generated elements) about the digital environment and/or computer-generated elements to determine a user's performance (e.g., whether the user is following the track, whether the user has properly framed the target, and/or whether the user has collided with an object).

In various embodiments, updates to the position of the simulated laparoscope can be obtained and calculated at regular intervals (e.g., 60 times per second). However, in various embodiments, only the positional information of the laparoscope is retrieved and used to update the digital environment. Any subsequent calculations related to the user's performance, in various embodiments, is generally performed using data extracted from the positional information as well as the stored information about the digital environment or its computer-generated elements associated with the selected camera navigation exercise.

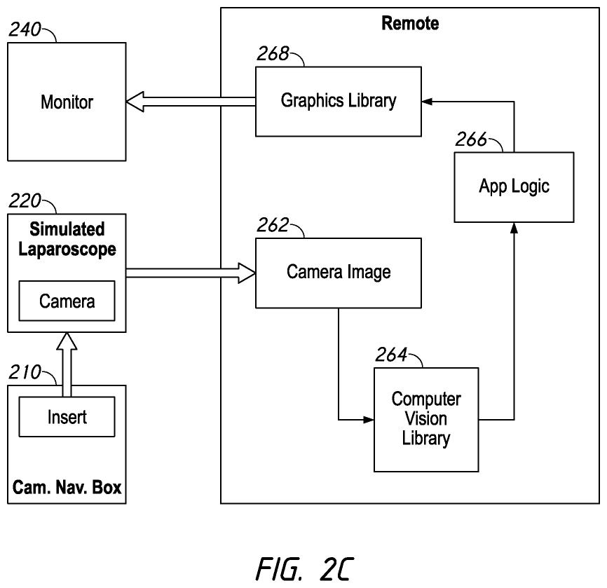

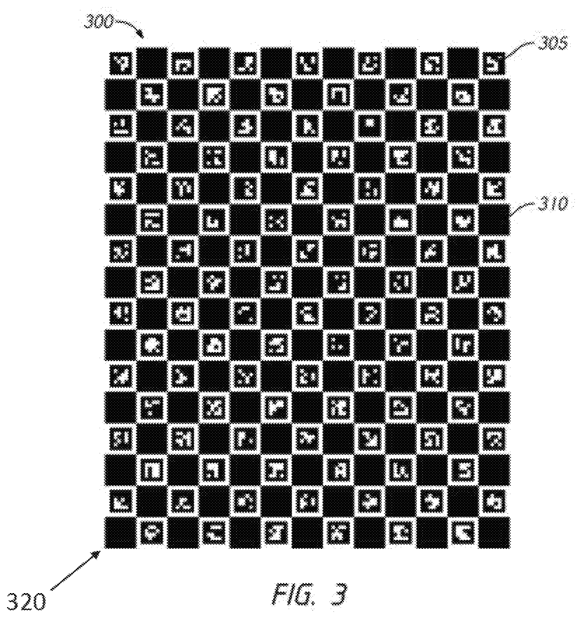

With reference to FIG. 3, the figure illustrates an embodiment of the insert or grid (300). The insert or grid (300) comprises a specialized arrangement of markers that may be positioned on part of or the entirety of the floor, or other internal surfaces (e.g., side walls, and/or ceilings) of the surgical trainer and/or camera navigation box. As seen in FIG. 3, the markers (305) are displayed in a checkboard arrangement with the markers occupying light squares and alternating with dark squares (310). The dark squares (310) are provided to space apart adjacent markers (305) and to allow easier identification of the individual markers (305). In various embodiments, the arrangement may use a different arrangement than the aforementioned checkboard based on the shape and size of the markers used. For example, the insert or grid (300) may remove all the black squares and instead have all the markers be placed adjacent to each other. One benefit of having all the markers be placed next to each other is to reduce an area which would need to be image captured in order to have a minimum number of markers to be identified.

In various embodiments, the insert or grid (300) or markers may also be applied onto physical objects (e.g., objects that are placed on top of the insert or grid (300)). Such embodiments could be provided with the ability to distinguish the markers (305) associated with the base and/or sidewalls of the surgical trainer and/or camera navigation box from the markers associated with the object placed on the training environment so that the camera navigation system is able to distinguish between the surfaces of the training environment and of the object.

In various embodiments, the insert or grid (300) may be constructed as a planar surface or sheet comprising the specialized arrangement of markers (305). Depending on the desired size of the training environment, the insert or grid (300) may be expanded to encompass part of or the entirety of the corresponding internal surfaces of the camera navigation box, such as the entirety of the base of the camera navigation box. In various embodiments, the insert or grid (300) may be positioned only on a portion of the camera navigation box and/or surgical trainer. Such embodiments would allow the camera navigation exercise to direct the user to move the simulated laparoscope within a more restricted area.

In various embodiments, the specialized arrangement of markers used in connection with the insert or grid of the training environment may comprise a plurality binary square markers (e.g., QR (quick response) codes). In various embodiments, the markers (305) can instead be integrated (e.g., printed) onto the internal surfaces of the camera navigation box, surgical trainer, and/or the like, and/or on the objects housed within the camera navigation box, surgical trainer, and/or the like. In various embodiments, the insert or grid (300) is removable relative to the camera navigation box, surgical trainer, and/or the like. In various embodiments, the markers (305) may be printed on one or more separate sheets; the sheets may be planar and/or have at least one surface flat or planar relative to the camera sensor of the simulated laparoscope. In various embodiments, the sheets facilitate the insert or grid (300) to be removable with respect to the camera navigation box. In various embodiments, the same insert or grid (300) can be used in a variety of different camera navigation boxes and/or surgical trainers. In various embodiments, different inserts or grids (300) can also be provided, created and/or used. In various embodiments, an insert or grid (300) can be provided and used without a surgical trainer and/or camera navigation box. In various embodiments, the camera navigation system aims to allow for the simulation of different actual surgical procedures or the practice of different surgical skills relying on tracking the simulated laparoscope or a similarly equipped instrument, tool, or accessory arranged to capture image data with reference to the insert or grid.

Although the markers (305) used with an insert or grid (300) may have a square shape as described in the various embodiments herein, it is possible to have the markers (305) have any number of different shapes such as triangles or circles. Whatever the shape of the markers (305), such information about the markers are stored in memory and usable to calculate the positional information of the simulated laparoscope.

Although various embodiments described within this application are described as using QR codes as the unique markers to identify the position of the simulated laparoscope within the training environment (e.g., camera navigation box), other symbols can be used so long as each and every symbol is unique. With different shapes and/or symbols used, the camera navigation system would need to be specifically designed to accommodate the different arrangements so that the data can be properly processed to accurately determine the position of the simulated laparoscope within the training environment. Furthermore, such information about the markers would be stored in memory and retrieved to determine the positional information of the simulated laparoscope.

As discussed above, each of the markers (305) used in connection with the training environment for the camera navigation system are unique from all other markers (305) associated with the same training environment. This allows for the appropriate identification of the simulated laparoscope's position within the training environment. In various embodiments, the location of a marker within the training environment can be categorized via an x, y, z set of coordinates. Each marker would have a unique set of coordinates which helps pinpoint the marker's location within a 3D space defined by the training environment. Each of the locations of the markers (305) on the grid (300) are stored in memory so that when one or more of the markers (305) are later identified using computer vision, the camera navigation system is able to pinpoint the location of where the simulated laparoscope is directed towards. The camera navigation system can then calculate, based on the image data of the markers, the positional information for the simulated laparoscope.

With respect to the digital environment, the 3D space associated therein corresponds to (e.g., is the same as) the 3D space defined by the training environment. Thus, for each marker that is defined with the training environment, a similar if not the same point would exist within the digital environment with both three-dimensional space having the same point of reference (0,0,0) coordinate. Thus any marker associated with the training environment having an x, y, z coordinate; the same point would exist within the digital environment having the x, y, z coordinate. These two points, between the training environment and the digital environment, correspond to each other.

For the purposes of illustrating the correlation between the training environment and the digital environment, and defining the three-dimensional space for these two environments, we note the following scenarios:

In various embodiments where the markers (305) are arranged on the base of the camera navigation box, the coordinates of the markers (305) are stored in memory using the reference point (320) at the bottom left corner of an insert or grid (300) (i.e. corresponding to an (x, y, z) coordinate (0,0,0)). Each of the markers would have an x, y coordinate pair with z=0. The same would then be true with respect to the digital environment as the cursor locations on any of those same spots would share the same x, y coordinate pair with z=0.

In various embodiments where the markers (305) are arranged on either of the side walls (such as those illustrated on 104a and 104b in FIG. 1G-2) of the camera navigation box, the markers have the same x coordinate value with y and z being variable. In fact, the x coordinate value for the markers on side wall 104a would be 0. Similarly, the markers located on the other side wall 104b would have all the same x value (but differ from the x value for markers on side wall 104a since they are away from the point of reference (0,0,0)) with variable y and z coordinates. With respect to the digital environment counterpart, the points within the digital environment corresponding to those markers on the side walls 104a and 104b share the same coordinates (i.e. x, y, z).

In various embodiments, where the markers (305) are located on the back side wall (such as those illustrated on 104c in FIG. 1G-2) of the camera navigation box, the markers have all identical y coordinate values while the x and z would vary based on the placement of the marker on the insert or grid. With respect to the digital environment counterpart, the points corresponding in the digital environment associated with the markers on the back side wall (104c) share the same variable x and z coordinates with their y value being constant.

In various embodiments, where the markers (305) are located on the base of the camera navigation box (such as those illustrated on (102) in FIG. 1G-2), the markers have all the same z value and their respective x and y values would be variable based on their location. With respect to the digital environment counterpart, the points corresponding in the digital environment associated with the markers on the base (102) would share the same variable x and y coordinates but their z value being constant.

In other words, with respect to the arrangement of the markers in connection with the training environment, each marker has a unique identifier which corresponds to its specific location with reference to the reference point in the training environment. Furthermore, a corresponding location is also defined within the digital environment having the same specific location. Both the location within the training environment and the location within the digital environment would be defined by the same x, y, z coordinate.

In situations where the image being captured by the simulated laparoscope is not recognized or if the simulated laparoscope is directed towards an area without any markers, the camera navigation box can be designed to inform the user of the error. This can be done a few different ways. First, the user can be provided notification within the digital environment that the simulated laparoscope is not capable of being tracked and that movement back towards the training environment should be pursued. Second, the camera navigation may show on the monitor a real-time view of what the simulated laparoscope is currently viewing that's not the training environment. When seen, this is indicative for the user that the simulated laparoscope needs to be maneuvered back towards the training environment to resume tracking of the position of the simulated laparoscope. Furthermore, hints and feedback can also be provided for the user to direct where the simulated laparoscope should be moved to.

In some embodiments, the lack of any identifiable marker (e.g., if the user tries to direct the laparoscope outside of the training environment) can trigger an error and temporarily remove the user from the camera navigation exercise. In some embodiments, the camera navigation system can be configured to display the real-time images being captured from the simulated laparoscope instead of the digital environment as a way to notify that the simulated laparoscope is not directed towards the training environment.

In various embodiments, when two or more images of the markers (305) are captured by the simulated laparoscope, the captured image data can be analyzed and identified by the camera navigation system to determine the set of positional information regarding where the simulated laparoscope is currently positioned. In particular, the camera navigation system is able to determine the end point of where the simulated laparoscope is located within a three dimensional space associated with the training environment (corresponding to the end point of the camera sensor and characterized by a set of x, y, and z coordinates) and/or how the simulated laparoscope is arranged within the three-dimensional space associated with the training environment characterized by a roll, pitch and yaw value. As used herein, the roll value corresponds to a rotation (around a longitudinal axis of the simulated laparoscope), the pitch corresponds to an angle relative to the insert or grid, and the yaw pertains to rotation about a vertical axis (perpendicular to the longitudinal axis of the simulated laparoscope).

In various embodiments, the camera navigation system is able to calculate some of the values based on comparisons made between the image data and data about the markers stored in memory. For example, if the images of the markers (305) appear distorted, the camera navigation system is configured to determine the particular angle or pitch of the simulated laparoscope by calculation transformations between the distorted and normal or predefined versions of the same marker. In various embodiments, the camera navigation system utilizes stored transformation algorithms to process distortions and convert such information to an angle or pitch value for the simulated laparoscope. Further details regarding how the positional information for the simulated laparoscope (i.e. the six degrees of freedom) is calculated will be described in further detail below.

In various embodiments, the simulated laparoscope would need to capture a pre-determined minimum number of markers (e.g., 4) to provide enough information from the insert or grid for the scope view generator to calculate and determine a position of the simulated laparoscope within the 3D space defined by the training environment. In one example, the simulated laparoscope may be required to capture at least two markers within the same image. However, there may be embodiments where one marker is sufficient or embodiments where three or more markers are required. As more markers are captured within a same image by the simulated laparoscope, a more accurate determination can be made. For example, a determination could be more accurate if seven markers were captured within the same image versus if only two markers were captured within the same image.

However, there may be an upper limit as to how many markers can be captured and used to determine the simulated laparoscope's position. Specifically, the more markers that are captured also increases the amount of time that is needed to process all the markers so that the camera navigations system can determine the positional information for the simulated laparoscope for that period of time. In situations where the information about the simulated laparoscope's position has not been updated in time, the camera navigation system may need to drop the current processing and move onto the next updated set of information for the simulated laparoscope.

In various embodiments, an optimal size and number of markers implemented on the insert or grid may be dependent on the simulated laparoscope being used. A balance needs to be struck by allowing cameras associated with the simulated laparoscope to identify the markers at near and far distances. In one embodiment, the markers have a width of around 1.63 cm. This may allow a particular simulated laparoscope to produce clear images for viewing with the simulated laparoscope between a range of 3 to 7 inches away from the marker. The ranges may depend on the type of simulated laparoscope being used as well as other factors such as the size and number of the markers associated with the insert or grid. Outside of the desired range, the images being captured by the simulated laparoscope may appear blurry on the monitor. In various embodiments, blurry image captures of the markers may still be usable to discern the positional information of the simulated laparoscope. However, the use of such blurry image captures is not desired due to the possible errors or inability to identify the marker(s) that can arise which in turn provides less accurate determinations of the simulated laparoscope's position.

In various embodiments, a number of markers that can be captured in a same image by the simulated laparoscope can be dependent on the angle of the simulated laparoscope as well as the viewing distance from the insert or grid. In various embodiments, at least two adjacent markers are needed for determination of the simulated laparoscope's position (i.e. location and/or orientation). However, the more markers that are captured on top of the initial two adjacent markers would further improve the determination accuracy of the simulated laparoscope's position.

Another consideration related to the construction of the insert or grid is the total number of markers to be included. In one embodiment, the insert or grid can have 216 markers although other embodiments can have more or less. The number of markers is dependent, for example, on the space available of the training environment (e.g., the insert or grid) for the markers to be placed on, the size of the individual markers themselves, and/or how the markers will be arranged. In various embodiments, more markers can be used in connection with placement on the side walls and/or ceiling of the camera navigation box.

The tracking of the position of the simulated laparoscope becomes more stable with increasing number of visible markers. However, with more markers included, there could be more computation needed to be performed by the camera navigation system tied to the identification and distinction between the different markers. The increased amount of computation may affect the responsiveness of the overall camera navigation system on providing the information to be used to update the position of the simulated laparoscope within the digital environment. Thus, the number of markers used, in accordance with various embodiments, aims to balance the desired responsiveness and speed of identifying the markers with the stability afforded with using more markers.

In various embodiments, the markers are placed and/or integrated on a flat or relatively planar surface (e.g. the insert or grid) relative to the front face of the simulated laparoscope, lens and/or image sensor used to capture the images of the markers. Having one or more markers placed on an uneven/unleveled or curved surface could cause distortion of the markers as seen by the simulated laparoscope which may result in errors in the recognition of the corresponding markers and/or calculations of the positioning of the simulated laparoscope thereby introducing errors in the tracking of the position of the simulated laparoscope in the training environment. To ensure that the markers are placed on a flat or planar surface, various embodiments use a rigid board that will retain its flat or planar shape or assist in maintaining the flat or planar shape of the insert or grid.

In various embodiments, the markers may be located on the base of the camera navigation box and/or surgical rather than on an insert or grid. Furthermore, markers may also be positioned directly on the side walls and/or ceiling of the camera navigation box and/or surgical trainer. With additional markers on the side walls and/or ceiling, the simulated laparoscope can be used in connection with the camera navigation system to have increased opportunities to always be directed towards a trackable surface. This is especially useful when using other surgical devices and/or when the simulated laparoscope is an angled laparoscope. In various embodiments, the use of an angled or articulated laparoscope, for example, is designed to allow viewing of areas of a training environment that a zero-degree laparoscope, for example, cannot or is less capable of doing so. Furthermore, having markers on the ceiling and/or walls of the training environment could also facilitate other entry points with respect to the training environment (e.g., insert or grid) such as having multiple openings into the camera navigation box and/or surgical trainer instead of, for example, only from a top surface or ceiling. In various embodiments, entry points can be positioned directly opposite the surface or position of the markers are located. In various embodiments, having markers on a back or distal side wall of the camera navigation box and/or surgical trainer could allow tracking when using the camera navigation system in a simulated vaginal approach or procedure. In various embodiments, the simulated vaginal approach or procedure may be carried out by having the simulated laparoscope enter the camera navigation box and/or surgical trainer from a front or proximal wall as opposed from a top surface or ceiling.

In various embodiments, the markers used with the training environment are a specifically designed implementation from the use of an open-source computer vision library, OpenCV. In one embodiment, the markers are implemented using QR codes arranged, for example, in the checkerboard pattern as illustrated in FIG. 3. As mentioned, the markers can be any symbol so long as each symbol is unique from other symbols arranged on the insert or grid and recognizable by the camera navigation system. Furthermore, the markers can be arranged in other arrangements than the checkboard arrangement shown in FIG. 3. In various embodiments, the camera navigation device stores the location of each unique marker associated with the training environment into memory. Reference can then be made to the stored information regarding the correlation between the images of the markers being captured by the simulated laparoscope and the position with respect to the training environment.

In various embodiments, the simulated laparoscope comprises a camera/image sensor or is attachable to a camera/image sensor. The camera/image sensor is used to obtain image data. The image data can contain image captures of the markers from the training environment. The simulated laparoscope used in connection with the camera navigation system can be either a zero-degree laparoscope or an angled laparoscope (e.g., 30 degrees). Other embodiments may also include other/additional surgical instruments or devices outfitted with or attachable to a camera or image sensor. Such embodiments would allow visual tracking (as seen on the monitor) for the positioning of the surgical device with respect to the training environment. In various embodiments, frame rate, field of view, and/or image clarity may affect the performance of viewing and recognizing one or more markers to track a position of the simulated laparoscope by the camera navigation system. For example, in some embodiments, the frame rate of the camera sensor could be between 30 fps (frames per second) and 60 fps, with potential image stuttering when the frame rate falls below 30 fps and support of 60 fps or above for smoother motion graphics.

The field of view of the camera or image sensor used to capture the image data impacts the sizing of the various computer-generated elements being displayed within the digital environment. With a smaller the field of view, there is less of a capability of capturing the extremes of zooming in and zooming out because the image data of the markers being captured already takes up a significant majority of the existing visual area. Thus, objects being resized according to the amount of zooming in and zooming out may be limited.

Furthermore, image clarity as provided or defined by the camera or image sensor can affect the performance of viewing and recognizing the markers, which could affect the accuracy of identifying the position of the simulated laparoscope. Factors that could affect the image clarity includes the camera's resolution, depth of view, and shutter speed settings. In various embodiments, a resolution between 640×480 and 1280×720 is used. However, resolutions below 640×480 may have increasing amounts of jitter and shakiness in the monitor. Furthermore, resolutions above 1280×720 may require more computing power to analyze while providing diminishing returns in tracking stability.

In various embodiments, the camera's depth of view settings can be optimized for the training environment being used. If one or more parts of the insert or grid are outside of the range of the simulated laparoscope's depth of view, those portions of the insert or grid will be blurred and difficult to use for the purposes of tracking the laparoscope's position. However, any part of the insert or grid that is inside the simulated laparoscope's depth of view range will have sharper and higher contrast edges, resulting in a more defined tracking of the simulated laparoscope's position.

In various embodiments, the simulated laparoscope's camera shutter speed affects how much motion blur is captured in the image data. By increasing the shutter speed, the change in position of the simulated laparoscope between subsequent captured images would be reduced, resulting in less blurring. Any distortions in the image data will reduce the quality of the tracking of the simulated laparoscope's position.

The image data of the targets obtained by the simulated laparoscope is transmitted to the scope view generator. In various embodiments, the image data can be transmitted via a wired connection (e.g., USB). In various embodiments, the information can also be transmitted wirelessly (e.g., Bluetooth). The scope view generator utilizes the image data from the simulated laparoscope comprising captured images of the markers obtained with respect to the training environment to generate or update the digital environment and/or generate or update computer-generated elements corresponding to the laparoscope's position. In various embodiments, the markers allow the scope view generator to determine the positional information (i.e. 6 degrees of freedom) for the simulated laparoscope with respect to the training environment. By tracking the position of the simulated laparoscope using the markers of the training environment, the camera navigation system allows the user to interact with the computer-generated elements, for example, to select different exercises as well as participate in the different camera navigation exercises by having a cursor movement correspond with the simulated laparoscopic movement. This is carried out by having the cursor within the digital environment mirror movements performed by the simulated laparoscope with respect to the training environment. By maneuvering the cursor to overlap with one or more computer-generated elements in the digital environment, the camera navigation system can identify user interaction with that computer-generated element.

In various embodiments, the cursor is an example computer-generated element that is used with the digital environment displayed on the monitor. The cursor location displayed on the monitor within the digital environment corresponds to the position of the simulated laparoscope with respect to the training environment. Specifically, the cursor is the point at which an imaginary ray extending from the distal end of the simulated laparoscope and parallel with the longitudinal axis of the simulated laparoscope intersects with the training environment. Described in a different way, the point where the cursor is located with respect to the simulated laparoscope would be similar to a scenario if the simulated laparoscope is replaced with a laser point; the point at the end where the laser pointer is directed towards a surface (which in this case is the training environment) is the same sort of point for the simulated laparoscope.

For illustrative purposes, we note the following observations pertaining to the position of the simulated laparoscope and the location of the cursor within the digital environment. The following three scenarios describe example relationships between the position of the simulated laparoscope with respect to the training environment and the location of the cursor within the digital environment.

First, when the simulated laparoscope is directly perpendicular to a marker associated with the training environment arranged on a base of the camera navigation box (such as (102) of FIG. 1G-2), the x-y coordinate value for the simulated laparoscope would be the same as the x-y coordinate value of the cursor within the digital environment.

Furthermore, using the same situation described above, the z coordinate value for the simulated laparoscope that is perpendicular to the marking arranged on the base of the camera navigation box (such as (102) of FIG. 1G-2) would be the same as the z-value for the cursor within the digital environment is when the simulated laparoscope is in contact with the training environment.

Lastly, in a scenario where the simulated laparoscope is perpendicular to the portion of the training environment arranged on the back side wall of a camera navigation box (such as on 104c of FIG. 1G-2), the z value of the simulated laparoscope would be equal to the z value of the cursor only when the simulated laparoscope is aligned perpendicularly.

In various embodiments, the simulated laparoscope could be inserted through a top or upper portion of a camera navigation box or top cover of a surgical trainer to simulate insertion of a laparoscope through an incision of a patient's abdominal wall. However, as discussed above, the simulated laparoscope can be inserted through different openings (e.g., front) to simulate other types of surgical procedures.

In various embodiments, the scope view generator comprises a processor and/or a computing device that is connectable with the simulated laparoscope. The scope view generator, in various embodiments, is configured with specialized local applications and/or web browser-based applications that would process the information obtained from the simulated laparoscope related to the markings on the insert or grid and/or output information related to the position of the simulated laparoscope relative to the training environment. The process, in various embodiments, entails the scope view generator using PnP processes to calculate information about the 6 degrees of freedom of the simulated laparoscope (e.g., x, y, z coordinates and roll, pitch, yaw). Exemplary computing devices may include laptops or desktops. Furthermore, the processor and/or computing device may be included or communicatively connected with a surgical trainer. The scope view generator may be communicatively connected to a monitor and the simulated laparoscope. Further details related to an embodiment of a scope view generator are shown in FIG. 2B. Please note that such an embodiment could correspond to a local implementation of the camera navigation system. Other embodiments are contemplated, for example, where the processing of the simulated laparoscope's position with respect to the training environment and/or the generation of the supplemental graphical elements can be performed remotely via cloud-based (e.g., via on the internet) and/or remote servers. Such embodiments are described (see FIG. 2C).

FIG. 2B illustrates an example dataflow for the camera navigation system illustrated in FIG. 2A. The steps performed by the scope view generator (230) can be performed locally (e.g. via a local processor, desktop, laptop, or the like), remotely (e.g., in the cloud or, via a remote processor or computing device (i.e. associated with a web browser-based implementation or application)), or a combination of both. The scope view generator (230) of the camera navigation system, in various embodiments, has various applications and/or access to libraries or stored data in memory that would facilitate generating and processing data associated from the simulated laparoscope and provide corresponding data for the digital environment and the computer-generated elements used for the various camera navigation exercise.

For example, as shown in FIG. 2B, a computer vision library (234) is provided. In various embodiments, the computer vision library (234) is a collection of programming functions that modify or analyze images being used by the scope view generator (230) to determine the positional data of the simulated laparoscope (220). In various embodiments, the images captured by the simulated laparoscope (220) correspond to images of the markers on the insert or grid. The computer vision library (234) is specially designed to utilize the markers of the insert or grid to track and identify the position of the simulated laparoscope within that three-dimensional space of the training environment.

The application logic (236) corresponds to a workflow logic where application data and logic is handled for the various simulated surgical exercises that are performable with the training environment. For example, by using the positional information (e.g., information about the 6 degrees of freedom) of the simulated laparoscope, the application logic (236) implements and executes virtual button presses, menu transitions, as well as any function needed to run the various camera navigation exercises (in which further detail will be provided below) by comparing the cursor location within the digital environment with the locations of the computer-generated elements within the digital environment.

As illustrated in the figure, the scope view generator (230) for the camera navigation system also has access to a graphics library (238). The graphics library (238) is specially designed to aid in the rendering of the digital environment and the computer-generated elements that will be outputted to a monitor for the camera navigation exercises. In various embodiments, the graphics library (238) may be dependent on the associated application logic (236) used to render the digital environments that will be displayed for the various camera navigation exercises. Exemplary embodiments may use various libraries such as OpenGL (Open Graphics Library) for desktop applications and WebGL for the web browser-based applications.

Returning to FIG. 2A, the camera navigation system (200) comprises a training environment (210) where tracking of the simulated laparoscope is performed. In various embodiments the training environment (210) is implemented via the insert or grid. In various embodiments, the insert or grid can be enclosed/housed within a camera navigation box and/or a surgical trainer. In various embodiments, the camera navigation system can work without the use of the insert or grid; rather the markers are associated with the internal surfaces of the camera navigation box, the surgical trainer and/or the like.

In various embodiments, the camera navigation box provides a controlled lighting environment for use with the insert or grid. In various embodiments, the controlled lighting environment is provided by one or more lights, such as LEDs, connectable with the camera navigation box and/or surgical trainer and configured to illuminate the entire insert or grid. In various embodiments one or more lights are controlled and/or activated by the scope view generator (230). In various embodiments the scope view generator (230) is capable of adjusting a brightness, orientation, and/or position of the lights. Unpredictable changes in the lighting environment can make tracking using the insert or grid much more difficult as the camera settings associated with the simulated laparoscope may dynamically change due to changes in the light conditions thereby altering the image quality. In various embodiments, the camera navigation box and/or surgical trainer can also provide a natural or fixed pivot point when the simulated laparoscope is inserted into the top or sides. A camera navigation system that does not use a simulated laparoscope or an enclosed structure such as a surgical trainer, such as a full virtual reality (VR) solution, could include a mechanically simulated pivot point.

In various embodiments, the camera navigation system may also include a lightboard to work in connection with the insert or grid. In such an embodiment, the insert or grid may be printed on a clear or transparent sheet that can then be illuminated from behind using a light source like the aforementioned lightboard. The light source would work with the cameras of the simulated laparoscope that have limited or no control over auto exposure which may bring about motion blur that impacts the ability to track the position of the simulated laparoscope using the insert or grid. With the lightboard, the camera and/or sensor associated with the simulated laparoscope can be flooded with light which could increase shutter speed. The result of the increased shutter speed could reduce the amount of motion blur captured by the simulated laparoscope.

In various embodiments, controlling the exposure settings for the camera for the simulated laparoscope and/or controlling the light source for the training environment is provided. However, in various embodiments, these actions may not be possible during an actual surgical procedure and may be strictly used only for simulating surgical procedures.

In various embodiments, the camera navigation system can utilize other types of markers aside from the QR codes described above in connection with the insert or grid. For example, unique, non-QR codes can be used. However, symbols used as markers must be unique and would need to be discernable from all other symbols associated with the training environment in that the camera navigation system can uniquely discern the position related to the symbol or combination of symbols captured by the simulated laparoscope. In various embodiments, the symbols (i.e. non QR codes) used as the markings on the insert or grid would be black and white with no gradient in between. Furthermore, a different specialized library (or further modification to the existing library) would include the non QR code symbols that would be specific to identify the symbols used in the alternative embodiment and any related information such as their specific location with respect to the training environment (e.g., x, y, z, coordinates). Furthermore, the new or updated library would be used in connection with the unique, non-QR code images obtained by the simulated laparoscope to help determine the position of the simulated laparoscope. Exemplary symbols that could be usable in these non-QR code embodiments may include various emojis, the alphabet, or photos of different objects.

In various embodiment, the insert or grid may be implemented using a computing device having a monitor. In particular, the computing device (such as a computer tablet) would have a monitor having a bright background. In various embodiments, by using a specially programmed application, markers could then be generated to be displayed on the monitor of the computing device. The simulated laparoscope can then interact with the markings generated on the monitor of the computing device acting as the insert or grid discussed above. In various embodiments, another similar embodiment could replace the computing device with a flat monitor that is connected to a computing device such as a desktop or a laptop and/or embedded with a processor. The connected computing device and/or processor would be configured to generate and display the markers onto the flat monitor which is designed to provide appropriate lighting for the simulated laparoscope. In various embodiments, a specialized display (e.g., a display with or attachable with memory) is configured to access and display the markers for the simulated laparoscope.

In various embodiments, the insert or grid may be replaced altogether with the use of a body form or enclosure (e.g., surgical trainer) having different shaped holes or thin spots that are backlit. The holes or spots would be designed to mimic what the tracking markers would be used for, therefore requiring each of the holes to have a shape be unique and easily distinguishable from each other. When the simulated laparoscope interacts with a particular hole or thin spot, the hole or spot would provide a response (i.e. reflecting light back to the simulated laparoscope). When the simulated laparoscope does not interact with a hole or thin spot, the space in the body form or enclosure may remain black. In such embodiments, the camera navigation system would include different applications and computer vision logic to properly identify what image is being captured within the body form or enclosure and translating that to a corresponding position of the simulated laparoscope.

In various embodiments, the camera navigation system uses the images of the markers captured by the simulated laparoscope to determine the simulated laparoscope's position relative to the training environment. In various embodiments, the camera navigation system is configured to receive and analyze the captured image data in order to generate and update the digital environment and its associated computer-generated elements. The digital environment and the computer-generated elements are displayed on the monitor. As described herein, the tracking of the simulated laparoscope is performed with the use of the insert or grid. The scope view generator uses the information coming from the simulated laparoscope alongside its computer vision library to help determine the position of the simulated laparoscope. Once the position is known by the scope view generator, this information is passed to the application logic which uses the positional information to evaluate whether any application-related actions should be performed such as button presses, menu transitions, executing camera navigation exercises as well as any other function needed to run and display the menu and/or camera navigation exercises for viewing. In various embodiments, the computer-generated elements displayed on the monitor are rendered using an open graphics library (e.g., OpenGL). The computer-generated elements can be rendered via a local and/or remote processor, a local and/or remote computing device (e.g., desktop/laptop) and/or remotely via a web-based graphics library (e.g., WebGL).

In various embodiments, the computer vision library (234) provides computer vision algorithms for the camera navigation system. The computer vision algorithms are used by the camera navigation system to determine the position of the simulated laparoscope relative to the insert or grid by identifying what marker(s) are currently being captured within the image data of the image sensor/camera of the simulated laparoscope.

The embodiments illustrated in FIG. 2A and FIG. 2B, for example, are generally associated with local implementations of the camera navigation system. For example, the camera navigation systems may be implemented at schools so, for example, so that students would be able to practice various camera navigation exercises in a classroom setting.

However, portions of the camera navigation system can also be implemented remotely (e.g., via the internet). As illustrated in FIG. 2C, the scope view generator can be performed remotely from where the training environment is physically located. In various embodiments, portions of the camera navigation system can be performed locally while other portions can be performed remotely. Further embodiments may also be possible where one or more steps for determining the simulated laparoscope's position can be performed both locally and remotely. As described herein, remote performance can be carried out on remote processors, computing devices, and/or servers at other physical locations separate from the training environment as well as via cloud-based servers (e.g., on the internet).

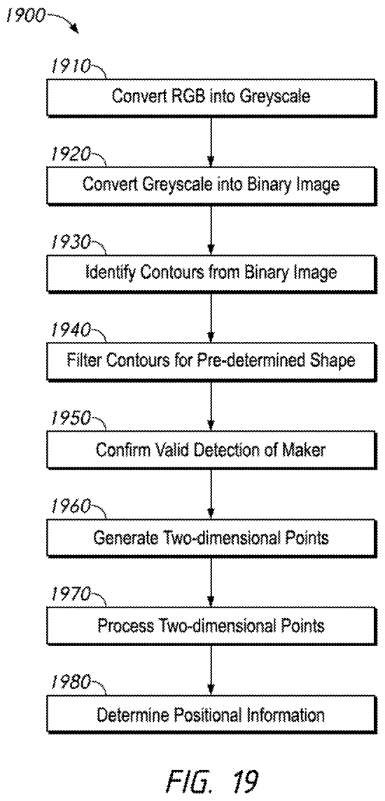

FIG. 19 illustrates an exemplary embodiment of the camera navigation system. In particular, the figure shows an example flowchart (1900) detailing the different operations that are used by the scope view generator to identify the simulated laparoscope current position relative to the training environment. In particular, the scope view generator identifies what markers are in the image data captured by the simulated laparoscope. The steps or operations include converting the captured image of the insert or grid obtained from the simulated laparoscope into a format that can be filtered and analyzed to determine the current position of the simulated laparoscope.

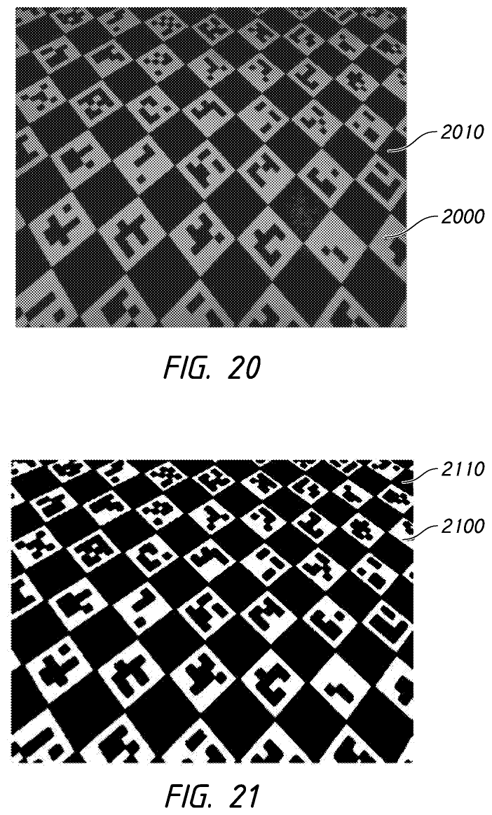

With reference to FIG. 19, once the scope view generator receives the captured image data from the simulated laparoscope, the scope view generator converts the captured images (which in many cases may be in color) from color (i.e. RGB) images into greyscale (1910). FIG. 20 illustrates an exemplary RGB conversion to greyscale. The figure shows the checkerboard arrangement of the plurality of markers (2000) with the black spaces (2010) that are used to space apart the plurality of markers (2000) apart from each other. Generally, the plurality of markers (2000) will be shown in a lighter coloration (e.g., white or grey), while the other spaces will be black (2010). In various embodiments, if the markers (2000) have nonwhite/black coloration, the camera navigation system may characterize different colors having a specific threshold so that the colors for the markers are converted to different shades of white, black, and grey. If the markers associated with the insert or grid are already in greyscale (i.e. black and white), there is no need to use the color information in future steps.

Once the captured image from the simulated laparoscope has been converted into greyscale, the scope view generator converts the captured image into a binary image (1920). FIG. 21 illustrates an exemplary binary image. As seen in the figure, the binary image has the plurality of markers (2100) and the black spaces (2110). In particular, FIG. 21 shows the end results of the conversion from greyscale into binary using adaptive thresholding. For each pixel associated with the binary image, the pixel is determined to either be fully on or fully off, fully on corresponding to the white portions and fully off corresponding to the black portions. The adaptive thresholding is a computer vision algorithm that facilitates determination of which pixel is “on” or “off” based on the greyscale image. Comparisons are made between neighboring pixels and differences that are greater than a pre-determined threshold are used to distinguish between pixels that should be “on” and pixels that should be “off.”

In various embodiments, a computer vision algorithm of the camera navigation system samples the pixels in the greyscale image, turns the corresponding pixel on or off based on comparing the value and evaluating whether its value is higher or lower than an average of its neighbors. With the use of adaptive thresholding, the binary image is capable of highlighting the bright and dark areas of the previous greyscale image. With the binary image, the camera navigation system can utilize this information to identify the captured image of the one or more markers captured therein.

With the binary image, computer vision algorithms of the camera navigation system are used to analyze the binary image to find contours associated with the bright areas (1930). The bright areas can be seen as corresponding to the group of pixels that are “on” as illustrated in FIG. 21. A pre-determined number of pixels adjacent to each other would need to be determined to be “on.” If the number of pixels is below a pre-determined threshold, this would correspond to contours that would be too small for use and can then be disregarded.

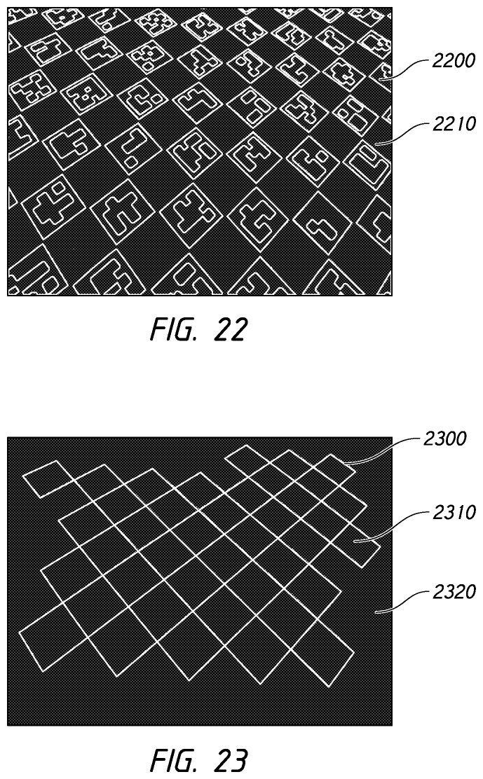

Each contour corresponds to a list of pixels that is used to outline the border of a bright area. Based on the location of the contours, the shape of each area within can then be calculated. FIG. 22 illustrates an exemplary contour calculation. The plurality of markers (2200) having the contours therein are shown in the same arrangement as the plurality of markers prior to the conversion. The black spaces (2210) between the plurality of markers (2200) are empty.

With the information associated with the contours of the bright areas, computer vision algorithms of the camera navigation system filters (1940) to only include quadrilateral shapes. FIG. 23 illustrates an exemplary filtering operation for quadrilateral shapes. Specifically, only contours or shapes that are convex and have four sides are considered to be possible markers of interest. As seen in FIG. 23, after the filtering has been completed, each of the quadrilateral shapes (2310) are shown in the image with each of the shapes (2310) having a corresponding outline (2300). Any portion of an insert or grid that is not recognized as having four sides, for example, if the plurality of markers or black spaces were cut off, are not represented after the filtering (2320). Comparing the image of FIG. 22 with the filtered image of FIG. 23, the contours located within the quadrilateral shape are ignored.

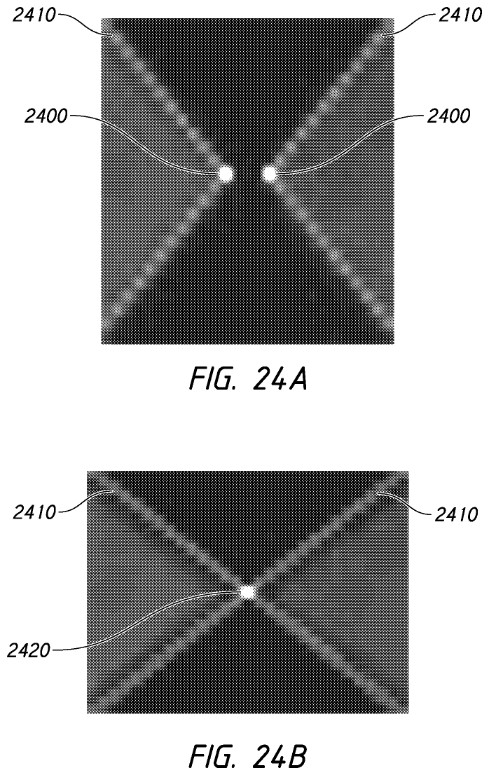

In a next step or operation, the corners (2400) and associated edges (2410) for each of the quadrilateral shapes can also be organized into a graph. FIG. 24A-F IG. 24C illustrate an exemplary filtering using corners of a quadrilateral shape. The graph used in the determination should only include corners (2400) that are likely to be one of the four corners of a marker. All corners associated with a marker will have two corners with connected edges (at 2420), as seen in FIG. 24B. However not all corners will be connected to other corners, as seen for example, in FIG. 24A. With reference to FIG. 24A, the corners (2400) are not connected; such arrangement would correspond to shapes that are not part of the quadrilateral shapes of the checkerboard arrangement and should not be considered.

The connected edges (2420) identify that they correspond a different markers arranged in the checkerboard arrangement. These adjacent corners are then merged to form one point with four edges (2410) which forms an “x” pattern (2420). An example merging of the corners can be seen in FIG. 24B. The camera navigation system identifies and removes any corners found inside the quadrilateral shapes as these are incorrect corners and do not correspond to the quadrilateral shapes (2430), as seen in FIG. 24C.

An end result with the filtering of the incorrect corners can be seen, for example, in FIG. 24C. In particular, the end result should have the quadrilaterals (2440) identified. The quadrilaterals (2440) that are primarily light-colored (i.e. on) correspond to the plurality of markers while the quadrilaterals (2450) that are primarily dark-colored correspond to the black spaces.

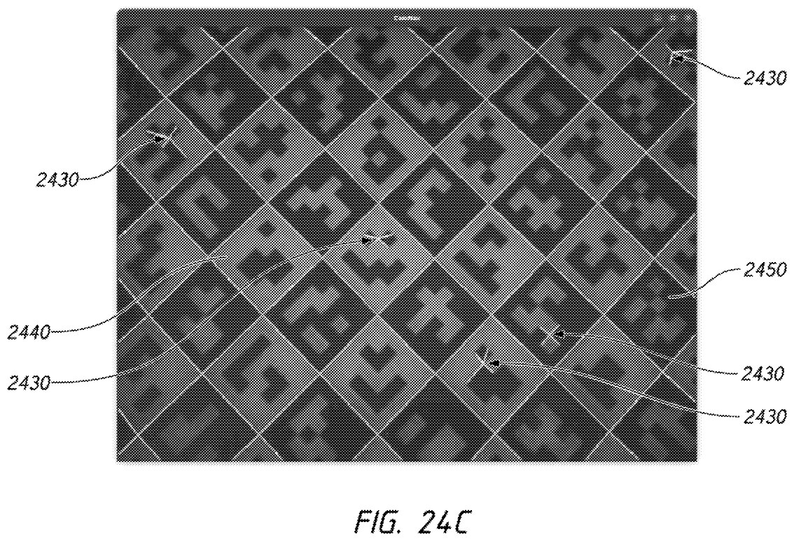

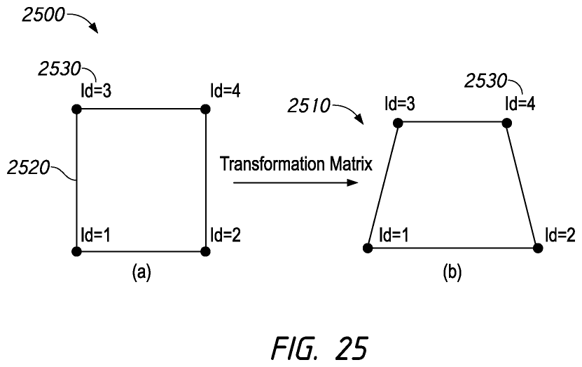

FIG. 25 illustrates an exemplary transformation matrix for determining distortion. In particular, the figure shows a transformation matrix (2500) that is performed by the scope view generator to properly identify the quadrilateral shapes associated with the markers on the insert or grid where distortion may be present. In various embodiments, the position of the simulated laparoscope will be such that a captured view of one or more of the markers (which are provided here as quadrilaterals) (2510) will appear skewed, e.g., when the simulated laparoscope is held at an angle with respect to the training environment. Since the pre-determined shape (e.g., square) (2520) that the markers captured by the simulated laparoscope are known and stored in memory, the camera navigation system is able to compensate for the skewing based on the predefined information regarding were each of the four uniquely labeled corners (2530) of the skewed shape (corresponding to Id=1 to Id=4) should be positioned as part of the pre-determined shape (e.g., square). The scope view generator solves a system of linear equations using the four unique points of the corners as inputs. The equations for the transformation generates a transformation matrix that represents the translation, rotation, scaling, and skewing needed to transform the skewed points of the skewed quadrilateral (2510) into the pre-determined shape (e.g., a square) (2520) that the markers should have. The transformation removes the distortion from the perspective of the simulated laparoscope, resulting in an image of the marker as if the simulated laparoscope were viewing, e.g., from directly above the marker or with the simulated laparoscope being aligned perpendicular to the insert or grid.

In various embodiments, other shapes are also possible, though their corresponding transformation matrix would need to be implemented. For example, shapes having more or less than 4 sides can also be used (e.g., circles) but different mathematical techniques would be used.

To verify that a specific contour (e.g., quadrilateral shape) contains a marker (1950), the pixels contained therein are sampled in a grid. A bright pixel represents a ‘1’ while a dark pixel represents a ‘0’. When all the 1's and 0's are combined, the combination forms a unique identification for a specific marker. If the identification matches a known marker (with information about the known markers stored in memory), then the contour is confirmed to be a valid detection. The “known” markers are based on all the markers stored in memory used to correlate to a specific location within the training environment. In various embodiments, the specific location is characterized as an x, y, z set of coordinates which pinpoints the marker within a 3-dimensional area associated with the training environment.

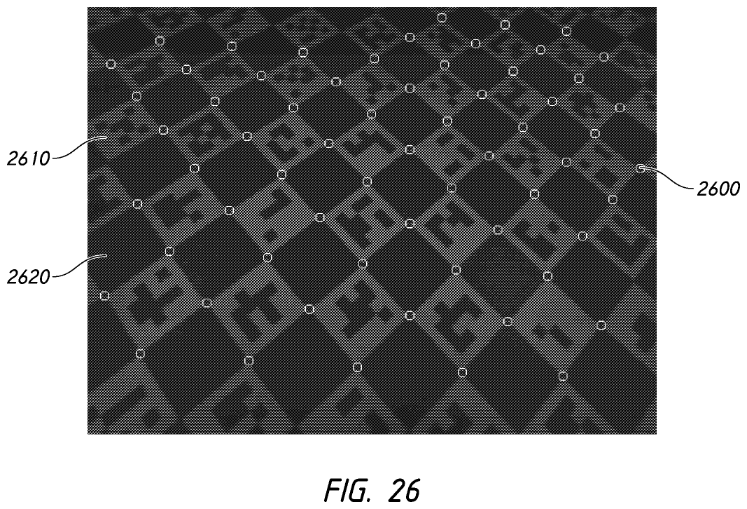

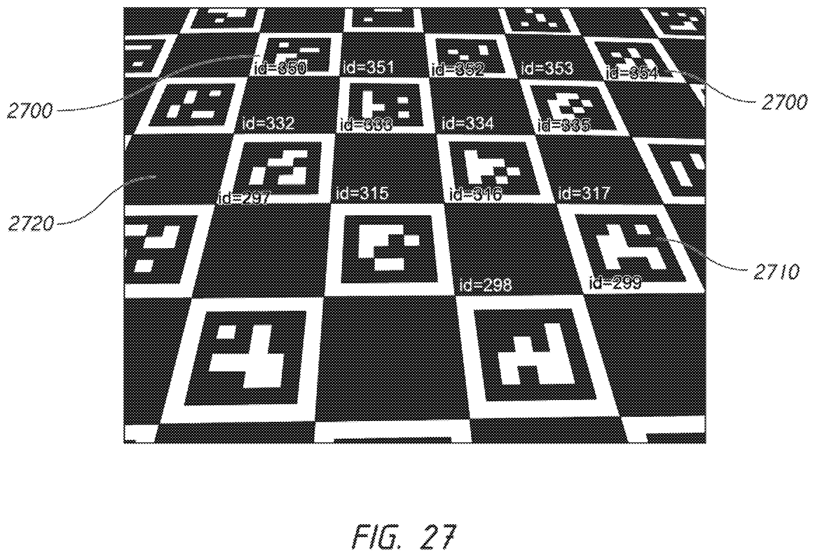

After the confirmation of the valid detection of a marker, the next step or operation being performed (1960) is to add corners (2600). FIG. 26 illustrates an exemplary operation of adding corner points; the figure specifically illustrates a corresponding set of two-dimensional points (1960). The corners (2600) are positioned at all the intersections of the edges formed by the plurality of markers (2610) and the plurality of black spaces (2620). The two-dimensional points will be used in another step or operation (1970) to determine positional information of the simulated laparoscope relative the training environment. Specifically, each marker has corner points with unique labels, for example, as seen in FIG. 27. By comparing the corner (2600) and their unique labels (2700), with the information about each of the markers stored in memory (which includes information about their respective unique corners), an orientation (e.g., roll) could be calculated.

FIG. 27 illustrates an exemplary step or operation of labeling each of the corners. As seen in the figure, each of the corners identified in FIG. 26 are labeled (2700). Each marker (2710) and each black space (2720) can be defined by a set of four uniquely labeled corners (2700). This is compared with the information about each of the corners of each of the markers stored in memory. As noted above, the offset of the respective corners is used to determine the orientation (i.e. how much rotation about the longitudinal axis of the simulated laparoscope is detected) between the marker captured via the image sensor and the supposed orientation of the marker. The offset corresponds to the “roll” value, which is one of the 6 degrees of freedom for the simulated laparoscope.

Once one or more markers (2810) has been confirmed, the scope view generator will proceed to perform calculations to determine positional information of the simulated laparoscope with respect to the training environment (e.g., insert or grid) and provide that information to the digital environment (1980). As noted above, an example two-dimensional points processing step is shown in FIG. 26. The process pertaining to how the position of the simulated laparoscope within a three-dimensional space can be obtained from the two-dimensional points of markers associated with a training environment is a problem that can be solved using a process known as “perspective-n-point” or (PnP). In various embodiments, the PnP problem is solved through an iterative approach based on the Levenberg-Marquardt algorithm.

In particular, the PnP process is specifically useful in the calculation of 6 degrees of freedom for the positional information for the simulated laparoscope; the 6 degrees of freedom covering the x, y, z coordinate of the simulated laparoscope as well as the roll, pitch, and yaw within the three-dimensional space associated with the training environment. The identification of the markers is used to determine the location (defined by an x, y, z set of coordinates) of the simulated laparoscope in the three-dimensional space defined by the insert or grid, the corners of each of the identified markers will be used (via the PnP process) to extrapolate the roll, pitch, and yaw values for the simulated laparoscope which would describe how the simulated laparoscope is oriented within the three-dimensional space pointing towards the markers. The roll value characterizes how much rotation is present for the simulated laparoscope about its longitudinal axis; the pitch value characterizes a relative angle of the simulated laparoscope with respect to the viewed insert or grid; the yaw value characterizes the rotation of the simulated laparoscope about its vertical axis.

The roll value for an angled laparoscope, in accordance with various embodiments, is determined in the same manner as previously described. However, in various embodiments, the roll value is further supplemented with a rotational value measured via a rotational sensor provided by or with the angled laparoscope. In particular, in various embodiments, the roll value calculated using the PnP process of the image data of the markers obtained by the angled laparoscope is further modified by the angle detected by the rotational sensor associated with the angled laparoscope. The further addition to the roll value calculated by the PnP process takes into consideration the complexity of the manipulations that are possible using an angled laparoscope where rotation of the camera/image sensor can be provided via two different points of manipulation. Exemplary details about the angled laparoscope are provided with respect to FIG. 31.

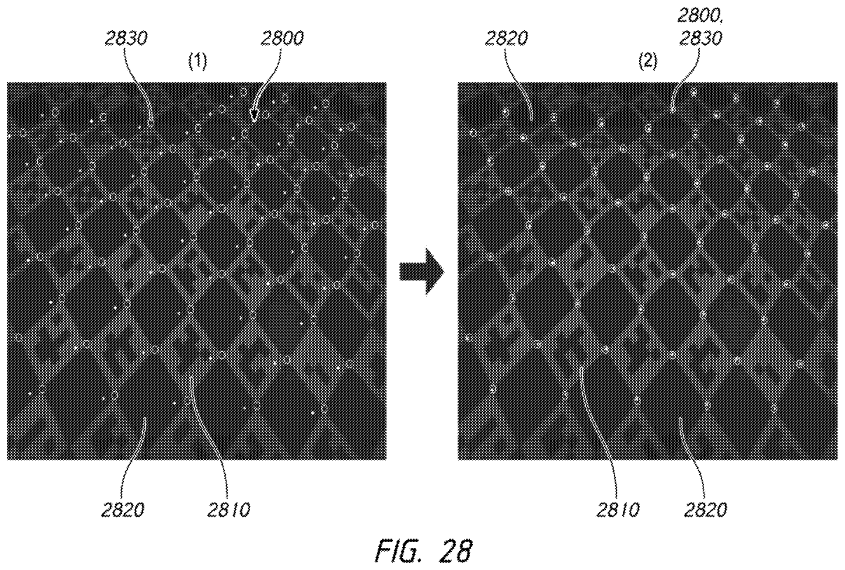

FIG. 28 illustrates an exemplary step of reprojecting the corner points with identified corners. As shown in the figure, the step moves the three-dimensional dots (2800) to match the two-dimensional points located at the corners (2830). Once the reprojected three-dimensional dots (2800) are matched with the two-dimensional points (2830) obtained in FIG. 26, the camera navigation system determines positional information about the simulated laparoscope within the three-dimensional space associated with the training environment by solving the information as the PnP problem. The points (2830) and dots (2800) used in this calculation are based on all the plurality of markers (2810) and black spaces (2820) captured in the image data. The more markers (2810) and black spaces (2820) captured, the more dots (2800) and points (2830) may be usable and provide a greater accuracy. The solution (e.g., obtained using the Levenberg-Marquardt algorithm) can then be used accordingly by the scope view generator, for example, in generating a corresponding cursor location shown with the digital environment displayed on the monitor. In various embodiments, the positional information for the simulated laparoscope can also be used to modify the digital environment to provide a different perspective (i.e. simulate the perspective from the simulated laparoscope). Furthermore, the positional information of the simulated laparoscope can be used to perform other calculations useful for camera navigation exercises such as determining whether a collision is present between the cursor and a target caused by other computer-generated elements (e.g., tube).

The monitor, in various embodiments, is used with the camera navigation system to provide a user interface through which users view the digital environment much like how surgeons rely on a monitor to view a surgical field within a patient during a surgical procedure. Through movements of the cursor that mimic movements of the simulated laparoscope, users are able to interact with the digital environment and computer-generated elements displayed therein. As noted above, the position of the simulated laparoscope relative to the training environment is correlated and shown as the cursor within the digital environment and subsequently displayed on the monitor. In this manner, users are able to use the simulated laparoscope and the monitor to select various camera navigation exercises from menus and perform those camera navigation exercises that are aimed at teaching and honing camera navigation skills useful or surgical procedures. In various embodiments, navigation through various menus and select options (e.g., via virtual button presses) is provided through the use of the simulated laparoscope. In various embodiments no separate hardware (e.g., controller, keyboard) or otherwise manual button presses would be required or provided. Rather, to carry out specific selection through the digital environment and computer-generated elements (via virtual button presses) or to perform actions related to camera navigation exercises, the scope view generator generates and displays a cursor (e.g. small circle or arrow) on the monitor. The cursor's location displayed on the monitor within the digital environment corresponds to the position of the simulated laparoscope relative to the training environment. To move the cursor to a different location in the digital environment that will subsequently be displayed on the monitor, a corresponding motion would be performed via the simulated laparoscope by the user. This correlation between the movement of the cursor shown on the monitor and movement of the simulated laparoscope simulates how a surgeon relies on the images on the monitor to maneuver the laparoscope and other surgical devices within the patient during an actual surgical procedure.

In various embodiments, the distance the cursor travels within the digital environment that is displayed on the monitor corresponds to the distance the simulated laparoscope is moved with respect to the training environment. In various embodiments, this correspondence can be different. For example, if the monitor provides a zoomed-up view of the digital environment, the movement shown may be more pronounced (i.e. two times or three times) on the monitor as opposed to the movements of the simulated laparoscope with respect to the training environment. The inverse is also true if the view is zoomed-out, the movements may be less pronounced (i.e. half, one-third) on the monitor as opposed to the movements of the simulated laparoscope with respect to the training environment. In various embodiments, notification can be provided to the user by the camera navigation system about the magnitude difference between the movements of the cursor and the corresponding movements of the simulated laparoscope.

In various embodiments, the cursor is used within the digital environment to represent the position of the simulated laparoscope relative to the training environment at any given time. In particular, the cursor represents the point of the training environment the simulated laparoscope is directed towards. The position of the cursor is based on the regular updating of the positional information about the simulated laparoscope obtained via the image data of the markers captured with in connection with the training environment being used. In various embodiments, the computer-generated elements (which are generated alongside the digital environment such as buttons, targets, menus, obstacles) are based on stored data associated with a selected camera navigation exercise or functionality (i.e. home page, menu). In various embodiments, the appropriate computer-generated elements are retrieved and displayed by the scope view generator based on the current state of use by the user. The cursor facilitates interaction with the various computer-generated elements (e.g., buttons). Such interactions are determined based on a comparison between the stored location of the computer-generated elements within the digital environment and the current position of the cursor within the digital environment. Specifically, the cursor is determined to be interacting with that computer-generated element if the positions for the cursor are within a pre-determined threshold associated with the location of the computer-generated element within the digital environment (i.e. overlapping).

An example embodiment of a computer-generated element is a menu (400) that can be seen in FIG. 4A. In particular, the figure illustrates an exemplary digital environment (405) that comprises a cursor (410) with various buttons (420-450) with which can be interacted with using the cursor (410). For example, the buttons (420-440) may include the various camera navigation exercises that can be practiced on using the camera navigation system. In various embodiments, these activities may include the focus activity (420), follow activity (430), and the trace activity (440). In various embodiments, these three exercises are usable for both the zero-degree and angled laparoscope.

To interact with the buttons (420-450) on the menu, the cursor (410), illustrated in the figure as a small circle, can be moved within the digital environment (405) to overlap at least a portion of one of the buttons (420-450). For example, as seen in FIG. 4B, the cursor (410) is currently overlapping the focus activity button (420). In various embodiments, the cursor (410) may appear as different objects/symbols such as an arrow, ‘X’or other shape.

In various embodiments, the camera navigation system utilizes the cursor (410) to facilitate user interaction with the various computer-generated elements (e.g., buttons, targets) in the digital environment (405) displayed on the monitor. In various embodiments, the cursor (410) may be pointed at or overlapping a portion of a specific computer-generated element. In various embodiments, the cursor (410) may also need to be held at the same position pointing at or overlapping the portion of the computer-generated element for a pre-determined amount of time. In various embodiments, the time requirement for an intended confirmation with a particular computer-generated element (e.g., button) may be set at two seconds. However, the pre-determined amount of time can be adjusted as needed. For example, the camera navigation system may confirm selection of elements faster or slower than two seconds. In various embodiments, the pre-determined amount of time can also be adjusted faster or slower accordingly to accommodate cursor interaction with computer-generated elements associated with different exercises that may require shorter or longer interaction time.

If the time required for selection of an element using the cursor is too short, it can cause unintended selections. In contrast, if the time required for selection of the element is too long, the user may lose interest or question if the element was selected properly. In various embodiments, a visual element (such as a status bar (460) as seen in FIG. 4B) can be used to show how much longer the cursor (410) must be held in the same position until the object (e.g., button (420) is selected. The status bar (460) can fade in and out as needed and fill up as the cursor (410) is held at a particular location. Once the status bar (460) is full, this can be used to indicate that the button (420) was selected successfully and also provide notification to the user that the button (420) was successfully selected. In various embodiments, the status bar (460) may reset or empty slowly if the cursor (410) is not at the appropriate spot for interaction with the computer-generated element (i.e. button (420)). However, when the correct position is reinstated for the cursor (410), the status bar (460) may resume filling up until full.

By using only a simulated laparoscope to interact with the digital environment and the computer-generated elements shown on the monitor, various embodiment are able to reduce the need to integrate additional hardware control devices such as keyboards or keypads with the camera navigation system which thereby simplifies camera navigation system and/or development process. In addition, using only a simulated laparoscope more closely simulates the actual camera navigation in a laparoscopic procedure where keyboards, mouses, and other hardware may not necessarily be used. If other supplementary control devices were used such as a keyboard or touchscreen, it would introduce non-immersive experiences that may not correspond with an actual surgical procedure such as pressing a key on a keyboard or tapping on a screen. Thus, the act of navigation and interacting with the computer-generated elements using the simulated laparoscope-controlled cursor as shown on the monitor is desired to provide for the immersive experiences that are more aligned with surgical procedures.

In various embodiment, to find buttons and read text as displayed on the monitor within the digital environment, movement of the simulated laparoscope with respect to the training environment is needed. For example, to select a virtual button being displayed on the monitor, the cursor within the digital environment must be held still over the computer-generated element for a certain duration of time. To move the cursor displayed on the monitor from one point to the desired location of the computer-generated element, the user would be required to move the simulated laparoscope a corresponding amount with respect to the training environment. Thus, even the act of navigating and interacting with the supplemental graphical elements can also be used as a form of camera navigation practice as the user would need to gauge distances on the monitor and move the simulated laparoscope a corresponding distance with respect to the training environment. Thus, training to understand the relationship between the movements shown on the monitor with the actual movements via the simulated laparoscope are also provided using the camera navigation system and its associated camera navigation exercises.

In various embodiments, when the application for the camera navigation system is initiated, a user interface can generate and display various different computer-generated elements which act as buttons that are associated with a variety of different exercises shown on the monitor. For example, as shown in FIG. 4A, three buttons (420, 430, 440) may be present that corresponds to three different exercises: trace, follow, and framing. Below these first three buttons may be an “exit’ button (450) that will allow the termination of the application when interacted with. When an exercise is selected using the cursor (410), the application can then subsequently generate and display a level/difficulty selection screen (470) as illustrated in FIG. 4C.

In various embodiments, the level/difficulty selection screen (470) will replace the menu (400) generated in the digital environment (405) to now show the available levels or difficulty (475) for that particular exercise. The scope view generator, upon receiving instructions based on the selected button (420, 430, 440) knows what next menu needs to be generated and displayed for the user. The scope view generator retrieves information about the necessary computer-generated elements and updates the digital environment accordingly, which in this case would be to provide the level/difficulty selection screen. Different users, identifiable by the camera navigation system, may have different progression on what levels or difficulties are available. Levels or difficulty availability for a particular user as provided and/or determined by the camera navigation system may be lit up (475) whereas levels or difficulties that are not yet usable can be darkened (476) and/or have a symbol (i.e. lock) (477) placed over indicating that the particular user cannot select the particular level or difficulty setting yet. In various embodiments, a pre-determined condition or proficiency (i.e. complete the previous level/difficulty and/or obtain a pre-determines grade/score on the previous exercise) tracked and/or enforced by the camera navigation system may need to be satisfied before being able to access higher levels or difficulties.

In various embodiments, the buttons associated with the different levels/difficulty as provided by the camera navigation system may preview or hint at the particular task to be performed. For example, as illustrated in FIG. 4C, the follow activity may include an image of an example path that will be practiced at a particular level/difficulty.

In various embodiments, the level/difficulty selection screen (470) as provided by the camera navigation system may also include “bonus” exercises (478). These “bonus” exercises (478) may not be required for completion of a course assigned by an instructor but are available to further test specific skills. In various embodiments, the “bonus” exercises (478) provide even harder challenges that users can undergo to hone related camera navigation skills.

In various embodiments, there may also be a “home” button (480) located on the user interface screen associated with an exercise. The “home” button (480), when selected provides the ability to return to the main screen. From the main screen, the buttons for the different exercises are shown again thereby allowing selection of a different exercise to practice on or quit the application altogether.

In various embodiments, once a difficulty or level is selected, the particular exercise begins. The scope view generator retrieves the related information about the exercise and computer-generated elements (e.g., targets, obstacles) to be used for that exercise. In various embodiments, the camera navigation exercise that is selected by the user will have corresponding computer-generated elements generated and displayed on the monitor for interaction during the exercise which facilitates the practice of skills related to camera navigation. The information related to the computer-generated elements that is stored in memory, for example, includes information such as their placements or movements within the digital environment. Thus, for example, selection of a particular camera navigation exercise will have the target located at the same location and any necessary information about the target (such as its location) can be retrieved as needed for the various determinations performed by the camera navigation system (e.g., whether a cursor is near or overlapping the target). In various embodiments, the use of the same location for the computer-generated elements for all users provides a “control” element that is useful for standardizing user performance feedback. As such, users are all provided the same scenario each time and are all graded using the same criteria. In various embodiments, the computer-generated elements may be provided with variable information (e.g., multiple possible starting positions), which could provide further challenges for the user to perceive and adapt to a given camera navigation exercise during each attempt. In the various embodiments, interaction with the computer-generated elements will require the simulated laparoscope to be manipulated and maneuvered with respect to the training environment.

In various embodiments, at various points during the camera navigation exercise, different options can be selected from a menu button. For example, navigation back to the home screen, navigation back to the difficulty or level select screen, and/or resetting and restarting the current exercise are provided and predetermined by the camera navigation system.

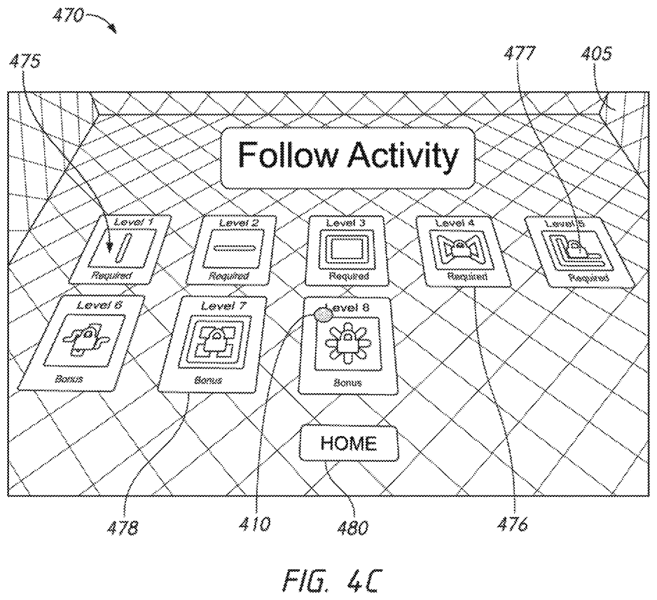

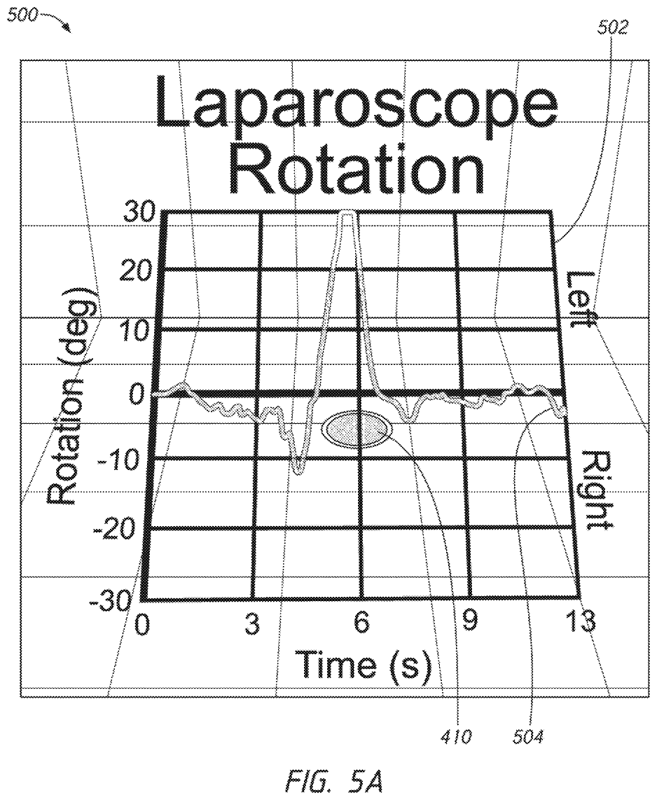

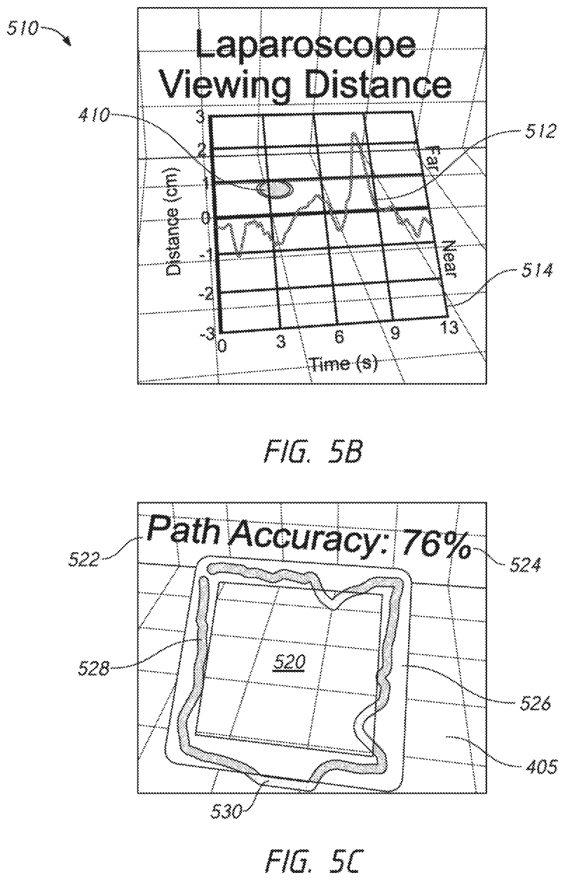

When an exercise is completed, in various embodiments, feedback screens are presented by the camera navigation system. FIG. 5A-FIG. 9 illustrate various embodiments of feedback generated by the camera navigation system that is displayed within the digital environment. The feedback screens, in various embodiments, provide summary information about the performance of the completed camera navigation exercise quantified on one or more criteria by the camera navigation system. Different exercises may have different predefined types of feedback. In various embodiments, the type of feedback can be focused on the skills that the user would like to concentrate on.

From the feedback screen, in various embodiments, the same exercise can be restarted from the beginning, movement to a different difficulty or level, returning back to the difficulty or level select screen, and/or returning to the home screen can be provided and predetermined by the camera navigation system.

During the performance of one or more different camera navigation exercises, various embodiments of the camera navigation system may provide animations during the camera navigation exercise. In many cases, animations can play a role in bringing attention to specific details. In various embodiments, certain prompts provided by the scope view generator to a user may not be recognizable nor the task at hand easily understandable. For example, a static yellow arrow may be used to provide directions as to where to start a new camera navigation exercise or where to go next. However, it may not be clear, without prior instructions or details, what the static yellow arrow represents or that interaction with the static yellow arrow is needed to progress to a next exercise. An animation may be provided by the camera navigation system with the static yellow arrow (e.g., oscillating the yellow arrow back and forth in the direction it was pointing) so that the arrow is made more noticeable as something to interact with.

In various embodiments, whenever there is a prompt for interaction, the camera navigation system provides some form of animation associated with the prompt. Another example of using animation would be to provide guidance in correcting the simulated laparoscope orientation during an exercise. For example, if the simulated laparoscope is rotated more than a pre-determined threshold or the horizon is not level, visual elements can be provided by the camera navigation system to inform the user that correction is needed and providing instructions, hints, or other computer-generated elements on how the correction can be performed (e.g., rotating the simulated laparoscope in the opposite direction to compensate for the previous rotation). The prompts can help in situations where prompts may be missed during an exercise. In various embodiments, the camera navigation system flashes the prompts in and out, the animated prompts can better draw attention to itself.

In various embodiments, prompts provided by the camera navigation system can also be provided in non-visual ways. For example, prompts can also be provided through audio (e.g., beeps) and via touch (e.g., haptic). These prompts could be used to provide information regarding whether the exercise is being performed properly or improperly. For example, if the cursor strays too far away from a pre-determine path or object, an audio sound or vibration can be used by the camera navigation system to provide notification of the occurrence of the cursor straying too far. Similarly, a different audio sound or different type of vibration can be used to provide notification of the occurrence that the cursor has properly acquired a target.

Audio prompts may include various sounds (e.g., beeps) or recordings that provide such status-based information described above. In various embodiments, the audio prompts can supplement the information provided from other sources (e.g., visual). However, the use of audio prompts may be based on the availability of speakers and could be influenced by factors (e.g., noise level) of the surrounding environment. Haptic elements provided, for example in the handle of the simulated laparoscope, could also be an alternate or complementary feature. However, implementing haptic elements could require additional hardware to be added, for example, into the simulated laparoscope. Furthermore, any vibration associated with haptic elements could directly influence (i.e. shake) the image being captured by the simulated laparoscope.

In various embodiments, the position of the simulated laparoscope is monitored through the location of the cursor within the digital environment to provide feedback on the user performance of one or more exercises. For example, the camera navigation system is configured to provide feedback on the performance of a camera navigation exercise including determining whether there was a rotation of the simulated laparoscope, whether the simulated laparoscope is at an appropriate viewing distance, whether the simulated laparoscope is accurately following a path, and/or the speed by which the simulated laparoscope is being maneuvered with respect to the training and/or digital environment. One or more of such criteria, in various embodiments, are evaluated during the performance on the different exercises by the camera navigation system. The following describe various example applications of the metrics used to evaluate performance by the camera navigation system. Other metrics could be used and are contemplated apart from what is described below.

In various embodiments, example metrics may include having the simulated laparoscope maintain centering of the operative field with respect to the training environment, keeping a target anatomy in view, and/or being able to follow the path of critical structures. This is quantified by determining a point of interest and detecting if the cursor is a pre-determined distance away from the point of interest. These metrics correspond to skills being practiced associated with maneuvering the simulated laparoscopic that would translate into being able to provide the surgeon during an actual procedure with an optimal view of the surgical site being operated on. Suturing and dissection often require a camera navigator maneuvering a laparoscope to zoom in so that the surgeon can clearly see small needles, tips of instrumentation, and individual tissue layers. Maintaining an operative field corresponding to an ability being taught and practiced on via the camera navigation system of maintaining the appropriate viewing distance with the simulated laparoscope. In procedures such as colectomies that require dissection through tissue planes, it is important to maintain the correct horizon to stay in the appropriate dissection plane corresponding to the ability to minimize the rotation of the simulated laparoscope. Suboptimal camera navigation during a procedure can lead to inefficiencies and increased duration for the operation corresponding to the ability to complete an exercise quickly and efficiently.

With respect to simulated laparoscope rotation, the camera navigation system monitors an extent (i.e. how many degrees) by which the simulated laparoscope twists or rotates around its axis. The amount of rotation may be graded based on the ability to maneuver the simulated laparoscope relative to the training environment without unnecessary twisting or rotation. Generally, the less the simulated laparoscope is twisted or rotated, the better the score. This is quantified by monitoring how much and how often the positioning of the simulated laparoscope changes during the duration of the exercise.

In various embodiments, feedback is visualized at the end of the related exercise being performed by the camera navigation system. An example rotational feedback (500) provided by the camera navigation system pertaining to rotation of the simulated laparoscope is illustrated in FIG. 5A. The rotational feedback (500) corresponds to a graph (504) that shows how much and how often the simulated laparoscope was rotated over a period of time during the duration of the camera navigation exercise as well as the direction (i.e. left/right or counter-clockwise/clockwise). The rotational feedback (500) tracks the rotation of the simulated laparoscope over time and identifies whether the rotation was to the left or right (502). Furthermore, different thresholds can be used and illustrated on the feedback as provided by the camera navigation system, informing where the amount of rotation is appropriate versus where an undesired amount of rotation was detected. For example, different colors can be used by the camera navigation system to indicate an appropriate (green) or excessive (red) amount of rotation during the performance of the exercise. In various embodiments, the camera navigation system provides user interaction with the rotational graph (410) with the cursor (410) in order to highlight further details about the rotational feedback (500), for example, what specific time period or by how much the rotation was detected.

In various embodiments, another criterion used by the camera navigation system pertains to the ability to maintain an appropriate viewing distance from a pre-determined target. The viewing distance uses a metric that measures the change in distance between the simulated laparoscope and the training environment. In particular, the “viewing distance” can be calculated from the positional information about the simulated laparoscope by solving for the “hypotenuse” of a triangle having the sides and corners defined by the information related to the x, y, z coordinates with respect to the training environment. The parallel within the digital environment has the viewing distance measure a distance between the position of the cursor and the position of the pre-determined target within the digital environment. The less change in distance during the course of the exercise, the better the score. To determine whether the simulated laparoscope is positioned at the appropriate distance, the current position (e.g., x, y, z coordinates) of the simulated laparoscope is monitored against the location of the target to determine if the simulated laparoscope is around the desired pre-determined viewing distance from the target.

After the exercise has been completed, the viewing distance feedback (510) can be generated and displayed as a graph. As illustrated in FIG. 5B, the viewing distance feedback (510) provides a graph (512) that shows a measure of how far the current location of the cursor is provided from the location of the pre-determined target (514) over the course of the exercise is shown. In various embodiments, the viewing distance feedback (510), provided by the camera navigation system, has the graph (512) that tracks the viewing distance over a period of time and quantifies whether the viewing distance is near or far (514) from the pre-determined target. Furthermore, different thresholds can be provided to inform what are acceptable distances compared to less desirable distances. For example, different colors can show the fluctuation in viewing distances and when a current distance at a point in time was too close, too far, or at a preferred distance.

In various embodiments, accuracy refers to how well the given task was completed. In the case of the Trace Exercise, accuracy refers to how well the location of the cursor stayed within a pre-determined path defined within the digital environment. As illustrated in FIG. 5C, a path accuracy feedback graph (520) is provided by the camera navigation system and associated with an exemplary exercise that may direct control of the simulated laparoscope to move the associated cursor along a pre-determined path. Once the trace exercise has been completed, the path accuracy feedback graph (520) may be generated within the digital environment (405). Determination of whether the cursor is along the pre-determined path is based on monitoring the location of the cursor over time and checking to see if the cursor corresponds to the stored locations associated with the pre-determined path. In various embodiments, the path accuracy feedback (520) plots out a path (528) shows the exact path the cursor took corresponding to how the simulated laparoscope was maneuvered. The cursor's path (528) may be overlapped with the pre-determine path (526) that was supposed to be taken. Colors or other indicators can be used by the camera navigation system to show when the cursor remained within the pre-determined path or left the path. In particular, if the path is within the pre-determined path (526), the path the cursor took (528) may be colored green. However, if portions of the path the cursor took (528) are outside the pre-determined path (526), such portions (530) may be highlighted or colored differently (e.g., red). Furthermore, the camera navigation system may provide the path accuracy feedback graph (520) that will label (522) identifying what the graph is displaying as well as a percentage (524) which summarizes how much the cursor stayed within the pre-determined path (526) during the entirety of the exercise.

FIG. 6 illustrates, in accordance with various embodiments, a user interface where the three types of feedback discussed above in FIG. 5A-5C can be combined into a single composite feedback display (600) in the digital environment. In particular, as seen in the figure, feedback for rotation (620), accuracy (630), and viewing distance (640) is provided along with the level difficulty identifier (615) used by the camera navigation system to indicate to the user what exercise was just completed. Each of the feedback graphs provided in the composite feedback display (600) have their respective plots which quantify the user's performance as determined by the camera navigation system with respect to their rotation (625), accuracy (635), and/or viewing distance (645) detected during the exercise. Such user interface would be viewable on the monitor.

Furthermore, in various embodiments, additional information (610) related to the shown composite feedback (600) can be provided in a pre-determined location within the digital environment (e.g., on the side of the current view of the feedback 600). For example, the additional information (610) can include threshold guidelines related to the different parameters used to evaluate performance of an exercise, a numerical grade/evaluation of that performance in comparison to the threshold, and the “highest” score that may have achieved in past performances. In areas related to the parameters being evaluated (e.g., rotation (620), accuracy (630), viewing distance (640)), the plots generated and provided by the camera navigation system can show where improvements were achieved as well as where improvements can still be made. An example identifier may include changing the colors of the graph (620, 630, 640) from green (corresponding to acceptable performance), to yellow (corresponding to performance that can be improved), and to red (corresponding to non-acceptable performance).

In various embodiments, at the bottom of the composite feedback display (600), may be various buttons (650, 660, 670) provided by the camera navigation system which users can interact with. A retry button (650) can be provided so that an exercise that was just completed can be repeated. A next button (660) can be provided so that the next exercise (i.e. a next difficulty) is provided or initiated. A home button (670) can be provided so that a main menu (400) is provided.

FIG. 7 illustrates another embodiment of the user interface of a composite feedback display (600) as shown in FIG. 6. The embodiment shares most of the same features already described earlier in FIG. 6, from the various different feedback for rotation (620), accuracy (630), and/or viewing distance (640) inclusive of their respective plots (625, 635, 645). However, instead of the additional information (610) that was displayed in FIG. 6 with the composite feedback display (600), the embodiment (700) illustrated in FIG. 7 includes a list of exercises where user performance of the associated skill has been “mastered” (i.e. the related performance completed the exercise above a pre-determined mastery threshold) (680) using the specific thresholds associated with the level or difficulty as determined by the camera navigation system. As seen with the mastery-related information (680) on the side, the grades would reflect a “mastered” score since the performance was within the acceptable thresholds for the parameters (i.e. rotation, accuracy, distance). In various embodiments, even if the scores achieved by the user are not “perfect,” if there is an improvement from past performances (i.e. a new high score) as determined by the camera navigation system, a corresponding message highlighting the user's improvement can be provided by the camera navigation system. For example, a notification (e.g., “great job”) and/or graphical visual effects can be provided via the user interface to inform that improvement was detected in one or more areas.

Depending on the camera navigation exercise, different types of feedback provided by the camera navigation system may be appropriate and thus used to quantify a performance during the camera navigation exercise. For example, as shown in FIG. 8, a further type of feedback may be used to evaluate the performance during a tracking exercise. In this figure, the feedback may quantify the amount of time that the location of the cursor was inside the locations defined by the pre-determined path compared to how long the cursor was outside the pre-determined path as determined by the camera navigation system. In another embodiment, with respect to the follow exercise, the accuracy criteria can include additional feedback data such as evaluating how well a cursor follows the target as the target travels along a path. The feedback can also be dependent on the type of path (e.g., whether the path may be pre-determined or random). In another embodiment, with respect to a framing exercise, the accuracy criteria can further be used to evaluate how steady the cursor is held in place when focused on a target.

As mentioned above, one criterion for evaluating a performance by the camera navigation system during an exercise pertains to monitoring a rotation of the simulated laparoscope. The rotation of the simulated laparoscope is measured in degrees by how much the simulated laparoscope twists around one or more axis. In particular, rotation can be measured with respect to the longitudinal and/or vertical axis. Having a rotation of zero degrees means the simulated laparoscope is aligned with the horizon (i.e. the planar surface of the insert or grid). In various embodiments, the horizon is defined as having a roll value of 0. This definition for the horizon generally corresponds to the bottom side of the image sensor used to capture the image data of the markers. This specific position is desired because a surgeon can most easily operate in this position. A rotation of 90 degrees means the simulated laparoscope is twisted perpendicular to the horizon. Arrangements of the simulated laparoscope not aligned with the horizon are generally not desired as it would make perceiving and maneuvering within the area more difficult.

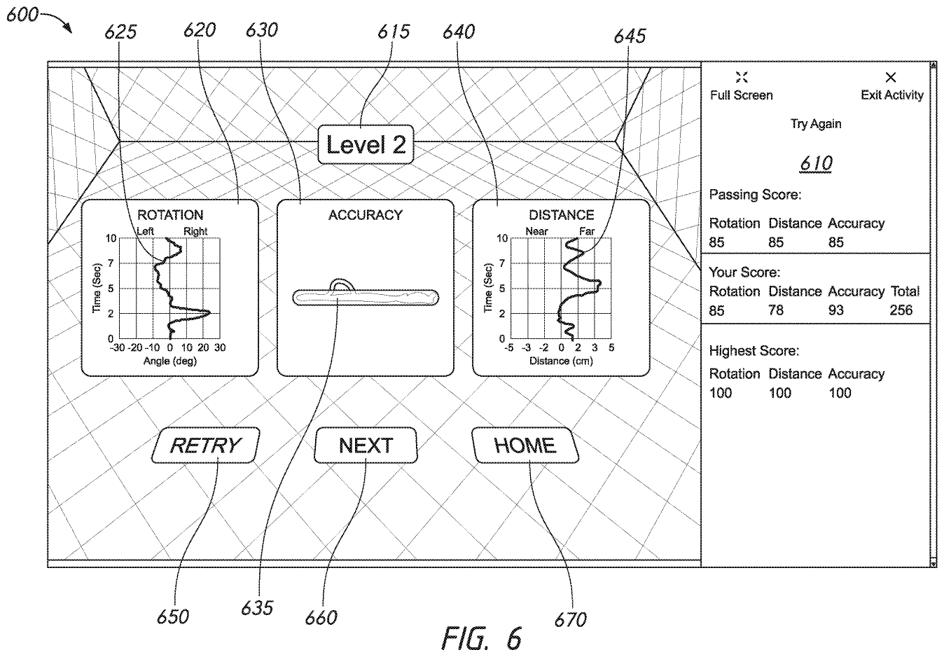

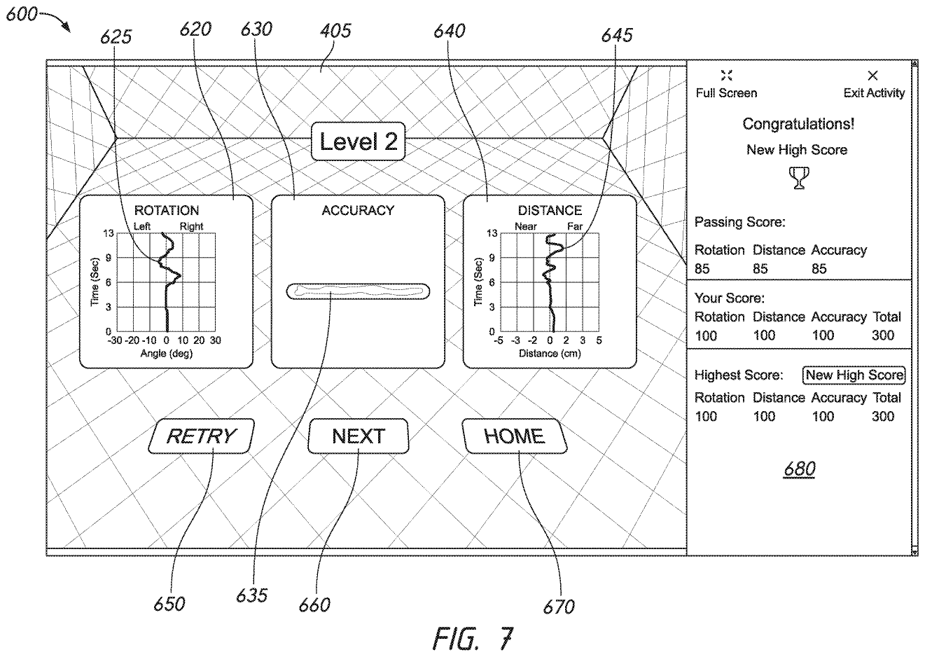

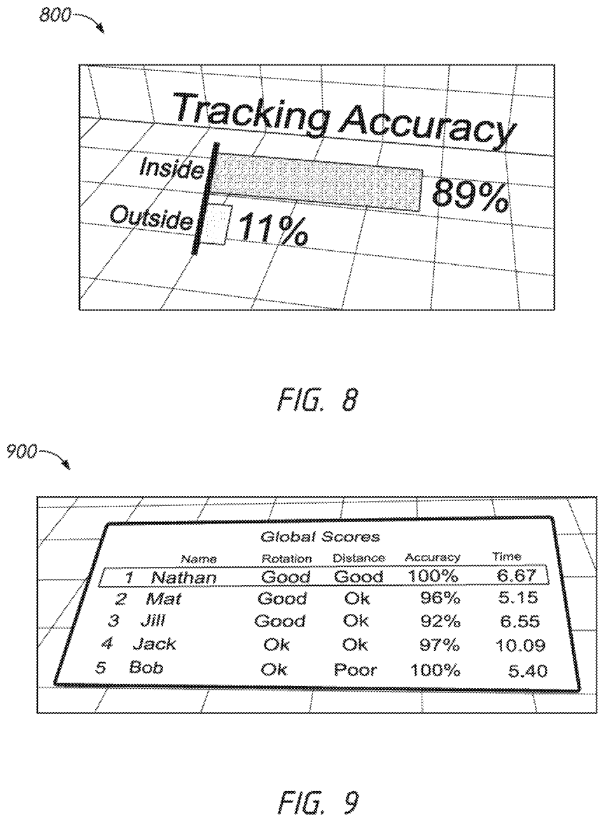

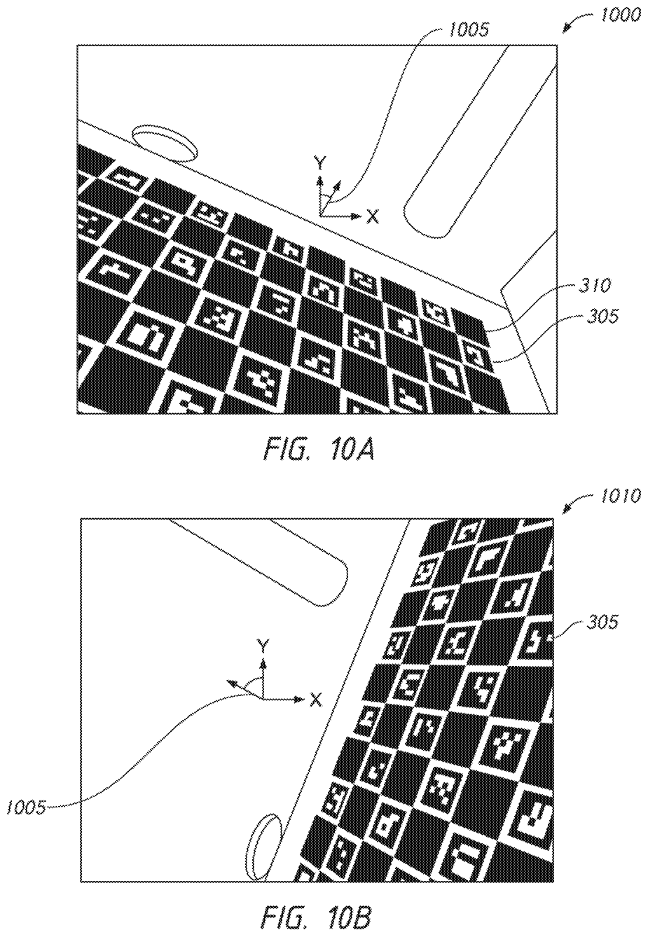

FIG. 10A-F IG. 10B illustrate exemplary calculations for determining a simulated laparoscope's rotation within the digital environment. The figures illustrate an exemplary image capture (1000) of the insert or grid. The image capture (1000) as received by the camera navigation system, would include the markers 305 and the dark squares therebetween (310). The image capture (1000) would utilize the x and y axis to determine the rotation. For illustrative purposes, FIG. 10A and FIG. 10B includes a super-imposed x and y graph (1005). Aside from the x and y axis shown on the x and y graph (1005) is a third line that represents a line that is perpendicular to the plane of the insert or grid. The simulated laparoscope's rotation is measured in degrees represented as the angle between the y-axis and the third line. This rotation is shown as the arc between the y-axis and the third line. This rotation metric is useful, as the aims of an exercise is to keep the simulated laparoscope positioning aligned with the horizon. Rotating the simulated laparoscope too far to the left or right during an actual surgical procedure can disorient the surgeon thereby possibly hindering the progress of the procedure. As seen in FIG. 10A and FIG. 10B, different positions for the simulated laparoscope used during the capture of the image data is shown. Specifically, the image capture of FIG. 10A shows a rotation that is too far right since the third line is between the x and y axis. Meanwhile, the image capture of FIG. 10b shows a rotation that is too far left since the third line is outside of the x-y axis. Ideally, the third line should be aligned with the y-axis to ensure that the image data is right-side up for the viewing on the monitor.

In various embodiments, the values used to quantify a performance during an exercise can be customized. For one embodiment, the camera navigation system may be configured to determine or grade a maximum rotation from the preferred 0-degree rotation whereby rotations of +/−30 degrees as “POOR”, +/−20 degrees as “OK”, and +/−10 degrees as “GOOD”. These numbers may change to adjust what can be evaluated as acceptable performance for different procedures or for different experience levels as determined by the camera navigation system. In various embodiments, the thresholds can also be based, for example, on the type of exercise being performed and/or the difficulty of the exercise (i.e. the harder the exercise meaning that the thresholds may be less forgiving for changes away from the 0-degree rotation).

FIG. 11 illustrates an exemplary embodiment of a meter (1110) provided by the camera navigation system to notify a user's performance. In various embodiments, a graphical element (e.g., meter) (1110) can be provided to appear if the simulated laparoscope is detected by the camera navigation system to have begun rotating to the left or right during the exercise. An example meter (1110) can be seen in the figure in connection with the maneuvering of the cursor (410) within a pre-determined path (1130). In various embodiments, different meters can be provided by the camera navigation system to quantify the user's performance for a variety of different criteria.

The meter (1110), in various embodiments, can have different zones (1115) that correspond to different grades (e.g., poor, ok, good). In various embodiments, the middle zone can correspond to ‘good’ performance. The next zones adjacent to the middle zone can correspond to a different grade of performance (e.g., ‘ok’ or ‘acceptable’). The last zone at the edges of the meter can correspond to a ‘poor’ grade of performance. These zones can also be color coded for easier reference by the user, for example, with green corresponding with “good”, yellow corresponding with “ok”, and red corresponding with the “poor” zones. A tracker (1120) provided by the camera navigation system can be configured to track or display where along the example meter (1110), the user is currently is in terms of rotation with the simulated laparoscope. If needed, for example, if the user is not in a desired rotational orientation, hints or directions (1125) may be provided by the camera navigation system to instruct the user to rotate in a particular direction. These hints (1125) would be helpful in directing the user to obtain the correct rotation for the simulated laparoscope since such information is not easily reflected via the cursor alone.

In various embodiments, as long as the measured rotation of the simulated laparoscope is controlled within the “GOOD” range of degrees (+/10 degrees) by the camera navigation system, the meter can be configured to be hidden. However, in various embodiments, if the simulated laparoscope is rotated even a little bit or rotates beyond that ‘good zone’ range into the other size zones (corresponding to ‘ok’ or ‘poor’), the camera navigation system can be configured to have the meter appear or displayed. In various embodiments, the meter indicates how far off the simulated laparoscope is from the desired orientation as determined by the camera navigation system. Furthermore, the meter can be used as a prompt or guide as how to take corrective action, such as how far to the left or right to rotate the orientation of the simulated laparoscope. By only showing the meter when needed, the camera navigation system can prevent or limit reliance/dependence on the meter to maintain the correct orientation since such meter would likely not exist or be present in a real surgery.

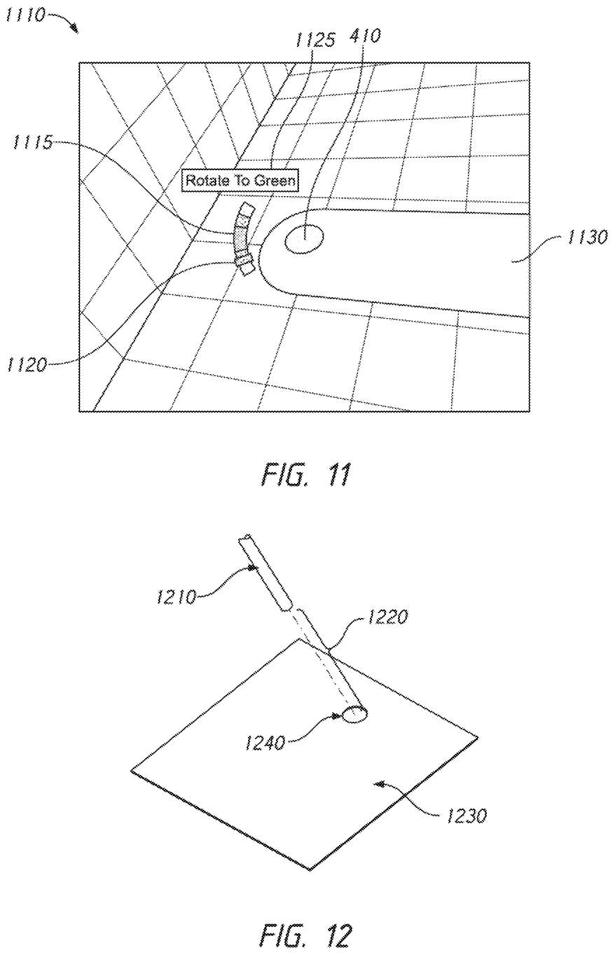

Another criteria used to evaluate a performance, in accordance with various embodiments, concerns the capability of maintaining an appropriate viewing distance between the simulated laparoscope (1210) and a target. FIG. 12 illustrates an exemplary calculation for viewing distance for the simulated laparoscope (1210) as determined by the camera navigation system. As illustrated in FIG. 12, the viewing distance (1220) being measured with respect to the training environment (1240) corresponds to the distance between the end of the simulated laparoscope (1210) and the training environment 1230) or a target area (1240). Depending on a procedure being simulated and/or the desired level or difficulty being practiced as determined by the camera navigation system, the desired viewing distances (1220) could be set to be smaller or larger. Furthermore, the different ranges and associated grade or performance qualification associated with the measured actual viewing distance of the simulated laparoscope can also be adjusted by the camera navigation system. For example, in one embodiment, a difficulty or level set or identified by the camera navigation system could have a measured viewing distance of +/−3 centimeters quantified as ‘poor’, +/−2 centimeters quantified as ‘ok’, and/or +/−1 centimeter quantified as ‘good’ as defined by the camera navigation system

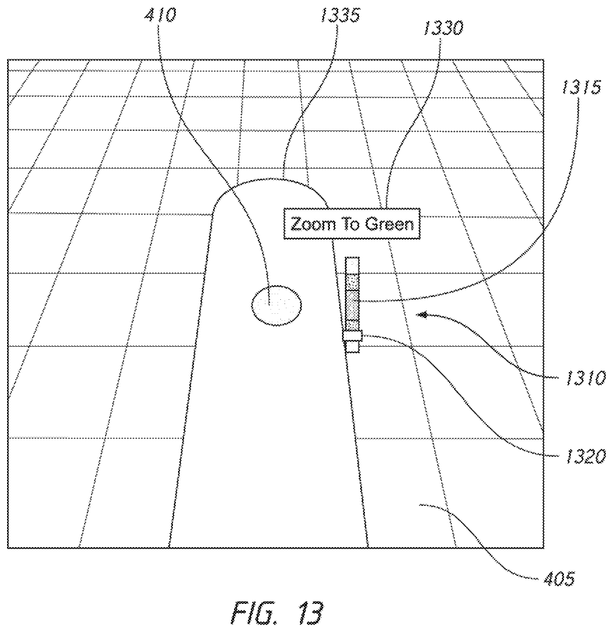

In various embodiments, if the simulated laparoscope is positioned out of the desired or ‘good’ viewing distance range during an exercise conducted within the digital environment (405), as determined by the camera navigation system, a range meter (1310) can be provided by the camera navigation system that operates similar to the simulated laparoscope rotation meter (1110) described above (as illustrated in FIG. 11). FIG. 13 illustrates an exemplary embodiment of a range meter. As seen in the figure, the range meter (1310) is configured to provide different zones (1315) corresponding to ‘poor', 'ok’ and ‘good’ performance/grade qualification. For example, the first middle zone can correspond to a ‘good’ performance; the zones immediately adjacent left and right to the ‘good’ zone correspond to ‘ok’ or zone; and the zones on the ends of the range meter corresponding to ‘poor’ performance/grades as it pertains to maintaining a desired viewing distance. A tracker (1320) as provided by the camera navigation system is provided to track the user's performance based on the meter; the meter can also have an associated prompt or direction (1330) that can be used as a guide to instruct a user how to return to the ideal viewing distance by moving the simulated laparoscope farther or closer to the target area within a pre-determined path (1335).

As discussed above, in various embodiments, there are various criteria (such as viewing distance and/or simulated laparoscope rotation) that determined and/or used by the camera navigation system to quantify and/or provide corresponding feedback during performance of camera navigation exercises. In various embodiments, the camera navigation system provides a variety of different exercises that will be aimed at training different skills associated with camera navigation during a surgical procedure. Described below are some example exercises such as target navigation, tracking moving objects, and image steadiness. In various embodiments, image steadiness is determined or quantified by the camera navigation system as the cursor being maintained in a predefined location for a pre-determined time period without changes or movements from the predefined location that exceed a pre-determined movement threshold. In other words, the camera navigation system determines that the image data being capture, whether to identify the position of the simulated laparoscope or in performance of one or more camera navigation exercises is steady and without significant shaking. Additional and different exercises are implementable in other embodiments of the camera navigation system that can be directed to other skills useful for camera navigation in general or for specific laparoscopic procedures. Furthermore, these camera navigation exercises, though described below in the context of applications with a 0-degree laparoscope, can also be applied to other types of laparoscopes (e.g., angled or 30 degree).

FIG. 4A-F IG. 4C illustrates various embodiments of computer-generated menus for the camera navigation system. n various embodiments, one or more different camera navigation exercises are provided by the camera navigation system and are selectable and/or performable via viewing on the monitor though a computer-generated menu (as seen in FIG. 4A). A particular level or difficulty for each camera navigation exercise can be provided by the camera navigation system and be selected by the user via the simulated laparoscope (as seen in FIG. 4C). In various embodiments, the camera navigation system may be configured to set or provide a level or difficulty that may have different metric parameters for evaluating performance of the exercise. In various embodiments, the level or difficulty provided by the camera navigation system may have different positioning requirements for the user relative to the training environment (e.g. standing on the side vs the front of the surgical trainer) which can provide a challenge to achieve the correct simulated laparoscope positioning (e.g., location and/or orientation). The camera navigation exercises, in combination with the simulated procedural setup, maneuvering of simulated laparoscope within a trocar, and experiencing the stick-slip friction and fulcrum effect associated with maneuvering the simulated laparoscope in connection with the surgical trainer, provide relevant and comprehensive dimensions of experience and practice for laparoscope operators outside of the operating room that are translatable for actual laparoscopic procedure during surgical procedures.

In various embodiments, an example camera navigation exercise that can be performed and provided by the camera navigation system is referred to as a ‘trace’ exercise. The ‘trace’ exercise measures how well the user maneuvers the position of the simulated laparoscope so that the corresponding cursor can be moved along a pre-determined path to specified locations within the digital environment. In practice, surgeons will often identify and follow a path of critical structures, such as along the ureter or vasculature, to avoid inadvertent injury or to help them identify other structures. Furthermore, it is common and best practice for surgeons to survey (via outlining) the overall operative field before and after the procedure to inspect for abnormalities. Thus, the tracing exercise described below helps improve skills such as dexterity, accuracy, and overall handling of the laparoscope since they are helpful for performing the above exemplary tasks as well as learning to overcome challenges imposed by the lack of depth perception, presence of stick-slip friction, and the fulcrum effect.

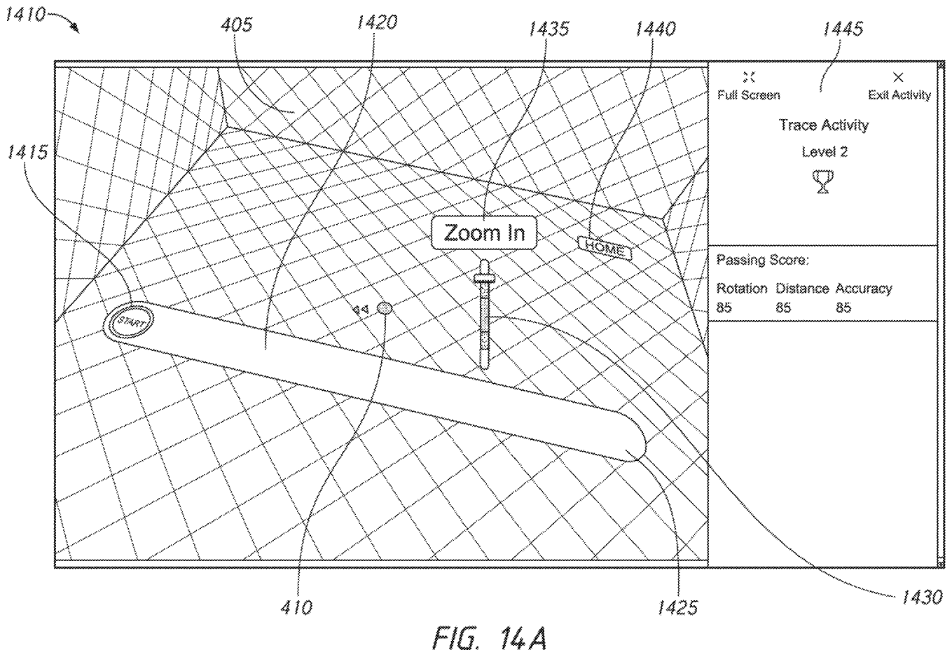

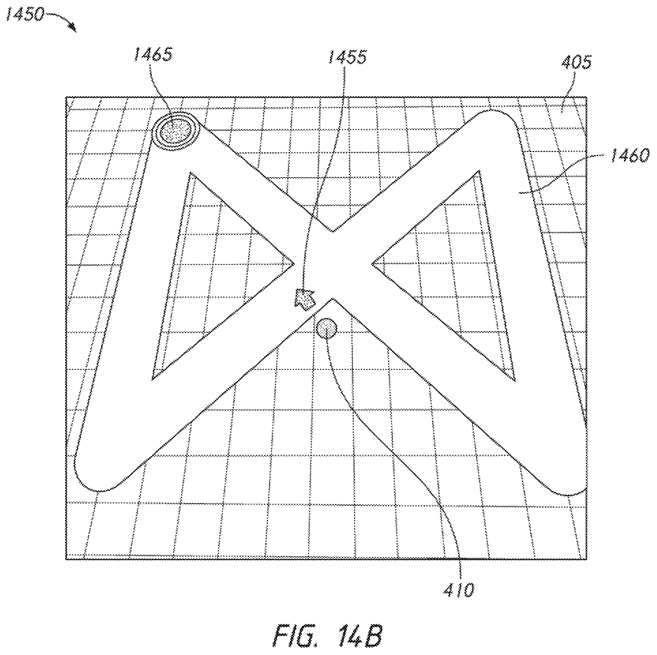

FIG. 14A and FIG. 14B illustrate an exemplary embodiment of a trace camera navigation exercise as provided by the camera navigation system. One goal of the ‘trace’ exercise is to have a user practice moving the cursor (410) along a set path (1420) from the beginning (denoted by a start identifier 1415) to the end (1425) while keeping the cursor (410) within that set path (1420) within the digital environment (405), generated, updated and/or evaluated by the camera navigation system. With reference to FIG. 14A, the figure illustrates an example embodiment (1410) of the ‘trace’ exercise as seen on the monitor. In various embodiments, instructions provided by the camera navigation system may be provided to maneuver the simulated laparoscope with respect to the training environment so that the corresponding cursor (410) moves within the digital environment (405) so that the cursor follows a specific path (1420). The trace exercise would help the user simulate a laparoscopic procedure of having the user practice outlining the operative field.

In various embodiments, when a level or difficulty is selected, the specific path (1420) that should be follow may be shown by the camera navigation system. Each level or difficulty may have different paths with different characteristics. In other words, the difficulty of the ‘trace’ exercise as predefined by the camera navigation system depends, for example, on the complexity of the path, the width of the path, and/or associated metrics used to quantify the performance (i.e. allowable amount of roll, allowable change in viewing distance) for the exercise. As seen in FIG. 14A, the specific path (1420) generated and displayed may be a straight path while another difficulty may have the path be more complicated as seen, for example, in FIG. 14B, with respect to the specific path (1460) corresponding to the exercise illustrated in the figure. In various embodiments, the specific path to be followed in the ‘trace’ exercise is stored in memory with the camera navigation system. Furthermore, the camera navigation system can also provide access to create new levels and/or modify old ones.

Performance of the ‘trace’ exercise has the camera navigation system regularly monitor the position of the simulated laparoscope with respect to the training environment to update the location of the cursor (410) within the digital environment. Comparisons are made by the camera navigation system between the location of the cursor (410) and whether the location is within the defined boundaries associated with the specific path (1420). Thus, the determination uses the stored information associated with the specific path (1420) and would be the same for each performance of the same camera navigation exercise whether by the user or other users. In various embodiments, the path may be randomized by the camera navigation system but maintain the same number of elements (e.g., length, number of turns, types of turns) to provide additional challenges for the user by not allowing the prior knowledge of what needs to be done for the exercise.

With reference to FIG. 14a, the ‘trace’ exercise may also provide a computer-generated element that serves as a meter (1430) which provides a gauge as to the user's performance on carrying out the ‘trace’ exercise. In particular, the meter (1430) can have different zones which can be used to quantify whether the user is appropriately executing the exercise or if the user needs direction. In various embodiments, remarks (1435) can be provided which informs the user how to better improve a performance of the current exercise. For example, in FIG. 14a, the viewing distance associated with the cursor (410) may not be within a pre-determined range. Thus, the remarks (1435) provided by the camera navigation system from the ‘trace’exercise may instruct the user to “Zoom In” with the simulated laparoscope.

The camera navigation system in various embodiments is configured to provide a ‘trace’ exercise that includes exercise-related information (1445) which provides additional information to the user about the exercise just as instructions to the user on how to perform the exercise and related thresholds for performance. In various embodiments, the user's performance (past and present) may also be displayed by the camera navigation system for reference.

In various embodiments, the ‘trace’ exercise may also include a ‘home’ button (1440). The ‘home’ button (1440) is configured to exit the current exercise and return back to the main menu or alternatively to the level/difficulty selection.

With reference to an embodiment illustrated in FIG. 14b, the ‘trace’ exercise (1450) as provided by the camera navigation system can include arrows (1455) that may be present that points to the start of that level or to the next point of interest (1465). The points of interest (1465) highlighted can include the “start” position for the exercise as well as any subsequent point the user is directed to move the cursor (410) to. This would allow users to know where the cursor (410) would need to be positioned in order to begin the level for the ‘trace’ exercise. In various embodiments, while the cursor (410) is proceeding to reach the starting point or the next point of interest (1465), the camera navigation system may have one or both the rotation and viewing distance meters shown. The use of the rotation and viewing distance meters would also be used to help set up and maintain the ‘trace’ exercise such as by ensuring that the simulated laparoscope is placed at a particular distance away from the surface and at an appropriate orientation. In various embodiments, before proceeding further with the exercise, the camera navigation system may prompt that the cursor must be in the appropriate (i.e. “middle”) zones of both the rotation and viewing distance meters to ensure that the appropriate starting conditions are satisfied. By initially showing both the rotation and viewing distance meters in real time, immediate feedback is provided by the camera navigation system on how to properly set up the starting conditions for the exercise. Furthermore, meters can be used now (and throughout the exercise) to indicate when errors in the rotation and/or viewing distance happen as soon as they occur so that the errors can be corrected accordingly via the simulated laparoscope.

In various embodiments, the user interface associated with this ‘trace’ exercise as well as any other exercise described herein (such as the ‘follow’ exercise discussed above and/or the ‘framing’ exercise discussed below) is configured have an information portion (1445) that includes details related to the exercise being performed. As seen in FIG. 14A, such information (1445) may include the selected exercise name, the difficulty/level being performed, instructions indicating what must done in the exercise to complete the exercise, and/or one or more guidelines related to one or more parameters being evaluated related to the performance of the exercise (e.g., rotation, distance, accuracy).

Once the cursor (410) is properly determined by the camera navigation system to be positioned at the start of the exercise, the first checkpoint is shown by the camera navigation system at some point along the path (1460). For example, as shown in FIG. 14B, a path (1460) being practiced on can have multiple turns and intersections for the cursor (410) to maneuver via the use of the simulated laparoscope. In various embodiments, the checkpoints (1465) can be shown by the camera navigation system one at a time to provide clarity on the goal and to prevent confusion as to where the cursor (410) would need to be maneuvered via the simulated laparoscope. In various embodiments, the checkpoint (1465) may be a colored circle. Furthermore, the camera navigation system, providing only one checkpoint at a time also simulates the inability for a surgeon to progress until an optimal view of the operative field is achieved. When the cursor (410) is successfully positioned over the checkpoint (1465) as determined by the camera navigation system, the camera navigation system provides that the checkpoint (1465) fades away and a new checkpoint is shown. In various embodiments, an arrow (1455) can be provided by the camera navigation system that points to the next checkpoint (1465). The arrow (1455) can fade away after a pre-determined period of time as determined by the camera navigation system or after the cursor (410) starts moving in the pointed direction. In various embodiments, a checkpoint (1465) cannot be collected if the cursor (410) is outside of the pre-determined path (1460) and/or is outside the acceptable simulated laparoscope rotation and/or viewing distance ranges.

In various embodiments, multiple checkpoints (1465) can also be provided simultaneously by the camera navigation system. The multiple checkpoints (1465) can provide an indication of an order so that knowledge of how to progress from one checkpoint (1465) to another along the set path (1460) is known. In various embodiments, the camera navigation system provides that a user is allowed to progress to checkpoints (1465) in any order so long as all the checkpoints (1465) are accounted for.

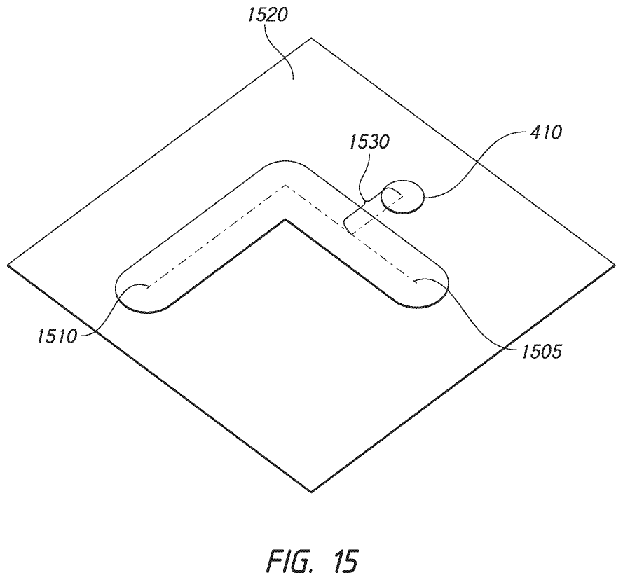

FIG. 15 illustrates an exemplary calculation for determining proficiency in the trace camera navigation exercise as determined by the camera navigation system. In various embodiments, the path traveled within the digital environment with the cursor is compared to each path segment along the pre-determined path to determine how well the cursor is maintained within the overall path. An exemplary analysis to determine how well the cursor is maintained within the overall path can be seen, for example, in FIG. 15. In an example embodiment, a center of the path (1510) associated with a pre-determined path (1505) is compared to the location of the cursor (410). The distance (1530) between the center of the path (1510) and the cursor (410) can be used by the camera navigation system to quantify the performance related to the ‘trace’ exercise performed within the digital environment (1520). When all the checkpoints along the pre-determined path are collected as determined by the camera navigation system, the level is completed. Afterwards, the overall feedback is displayed. In various embodiments, the accuracy of how a user performed the trace exercise is measured by the camera navigation system based on an amount of time spent inside the path (1505) compared to the time spent outside of the path (1505). In various embodiments, the extent by which the cursor moves outside of the pre-determined path (e.g., the measured distance 1530) may also affect calculated feedback related to accuracy.

Providing summative feedback, for example with the ‘trace’ exercise described above, is useful for assessing the overall performance. Furthermore, the feedback can be used to identify areas for improvement. In various embodiments, the feedback display as provided by the camera navigation system comprises of a variety of metrics used to quantify a performance of the exercise. For example, in various embodiments, the feedback display may include graphs exhibiting the rotation and viewing distance performance during the span of the exercise, a bar graph showing how accurately the cursor stayed inside the path over the course of the exercise, the time in which the exercise was completed, and/or leaderboards showing previous scores as well as scores of other individuals who have completed the same exercise. These metrics represent camera navigation skills needed in surgical procedures. Additional and different metrics may also be determined and included by the camera navigation system in the feedback display to further quantify the performance.

The data used for the feedback and/or leaderboards for the ‘trace’ exercise or any of the other exercises described various embodiments can be locally stored in memory for the camera navigation system. Thus, the feedback and/or leaderboards may be limited to use associated with a particular set up device or even within a physical location's network (i.e. hospital, school). However, in various embodiments where portions of the camera navigation system are implemented remotely (e.g. remote servers, internet), the data from all users can be stored remotely from the users and combined with users from all over. In various embodiments, implementations of the camera navigation system can also be possible where feedback can be presented that are based on the combined data of all who participated in the event on their respective devices. Leaderboards can include all data from all users, even those users that are physically distant from each other and who performed the same exercise on different trainers. Furthermore, various exercises can be performed at different physical locations the associated data can be maintained accordingly.

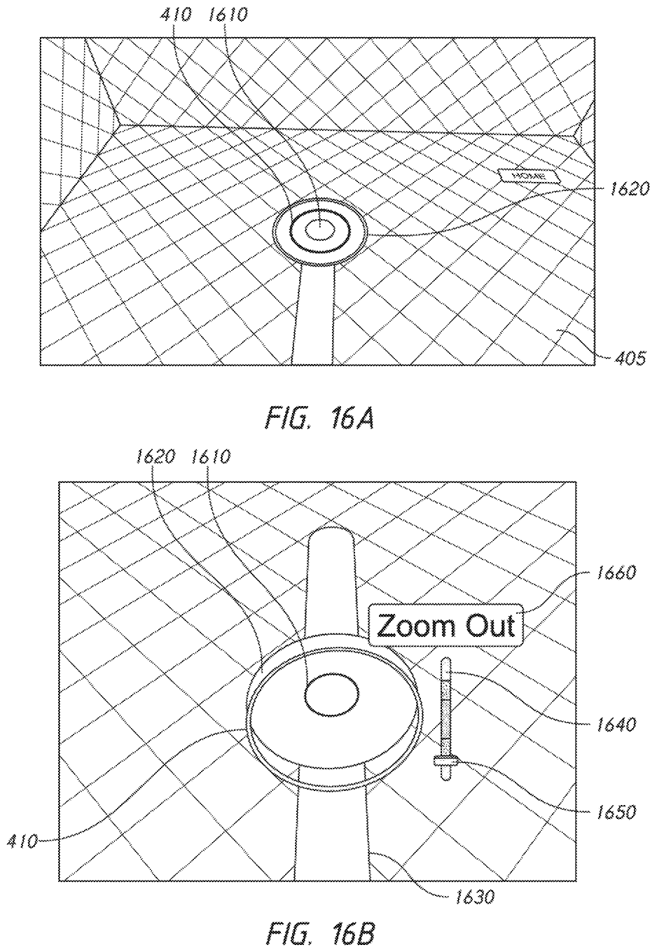

FIG. 16A and FIG. 16B illustrate an exemplary embodiment of a follow camera navigation exercise as provided by the camera navigation system. The ‘follow’ exercise is another exercise that may be provided by the camera navigation system that provides training in a different skill set compared to the ‘trace’ camera navigation exercise discussed above. In the ‘follow’ exercise, the camera navigation system is configured to measure the ability to control the location of the cursor (i.e., a ring) (410) within the digital environment (405) to follow a moving target (1610) which is denoted by a filled circle and a surrounding zone (1620) as seen in FIG. 16A. In some embodiments, the cursor (410) may need to remain within the surrounding area (1620), which is denoted an acceptable range to have the cursor (410) positioned within, associated with the moving target (i.e. the filled circle) (1610) and must not cross outside the surrounding area (1620) or the area denoted by the moving target (1610) for at least a pre-determined period of time.

In various embodiments, the camera navigation system is configured to begin the ‘follow’ exercise the same way as the ‘trace’ exercise with regards to identifying movement of the cursor (410) to a starting position and identifying or determining that the cursor is situated with an appropriate viewing distance and orientation. The starting position may need to be maintained for a pre-determined amount of time as determined by the camera navigation system and which can be illustrated or displayed by the camera navigation system via a meter (1640) filling or emptying. The meter (1640) may have various zones which can be used to quantify how well the cursor (410) is being positioned. A marker (1650) may be used by the camera navigation system to highlight where in the zones associated with the meter (1640) the user's performance is currently quantified as. Based on the user's performance, remarks or hints (1660) may be provided by the camera navigation system to help improve the user's positioning of the cursor (410). Once the initial starting position has been satisfied as determined by the camera navigation system, instructions can be provided to direct the cursor (410) to follow the moving target (1610) along the predetermined path (1630). In various embodiments, the camera navigation system moves the moving target (1610) along a pre-determined path (1630) at a set speed. The information for the pre-determined path (1630) is stored in memory associated with the particular level or difficulty for the exercise. In some embodiments, the target (1610) may move at variable speed and/or may start and stop at variable times; such features would change the difficulty of the camera navigation exercise being performed.

As noted above, example implementations of the ‘follow’ exercise are shown in FIG. 16A and FIG. 16B where the moving target (1610) is shown as a filled circle that will be moved along the path (1630). In various embodiments, the path (1630) may not be visible. In various embodiments, the moving target (1610) may move in a random manner that is not defined by a pre-determined path. However, to maintain continuity between different users using the same difficulty or level, characteristics associated with the path may remain the same; for example, having the same number of turns, starts/stops, etc. . . . The order for the features of the path may be re-arranged so that users are not able to memorize and anticipate the path but rather must respond and react accordingly. This skill is particularly important because a good camera navigator during a surgical procedure is able to predict the next moves of the surgeon for a particular surgical or laparoscopic procedure. The ‘follow’ exercise can improve the user's ability to anticipate and adapt movements for the cursor (410) with respect to the moving target (1610) all while maintain an optimal viewing distance. The ‘follow’ exercise thus would provide practice for a user anticipating the real-time movements of the surgeon and moving the laparoscope accordingly during a laparoscopic procedure.

In various embodiments, the camera navigation system is configured in that the moving target (1610) slows down or even comes to a complete stop if the camera navigation system determines that the cursor (410) moves (or has portions) outside of the surrounding area (1620) associated with the moving target. In various embodiments, the camera navigation system can also have the moving target (1610) slow down or even come to a stop if the rotation and/or viewing distance ranges of the simulated laparoscope is outside the acceptable ranges. This simulates the real-world condition that a surgeon must stop operating due to the inability to see associated with poor positioning of the laparoscope by the camera navigator. The thresholds associated with the acceptable values for rotation, location, and/or viewing distances can be adjusted, for example, based on the difficulty of the selected exercises. The thresholds may be smaller for harder difficulty exercises compared to thresholds being larger on the easier difficulty exercises.

In various embodiments, to inform that the cursor is not in the optimal view with respect to the moving target, visual indicators (such as the illustrated meter (1640)) can be used and displayed in the digital environment. Visual indicators, such as those discussed above in the ‘trace’ exercise can be used (for example, as seen in FIG. 11 and FIG. 13). With respect to the embodiment illustrated in FIG. 16B, an example meter (1640) can be used to show that the positioning of the cursor (in this case the viewing distance) is not within acceptable ranges. The meter (1640) may be shown next to the moving target (1610) which provides a gauge as to how far off from the acceptable range is. The meter (1610) may be split into multiple sub-sections each representative of distances away from the appropriate range for the viewing distance of the cursor with respect to the moving target. As the viewing distance (corresponding to an insertion depth of the simulated laparoscope) is adjusted, the meter (1610) can be adjusted accordingly to inform whether the adjustments to the simulated laparoscope has improved or worsened the viewing distance. A marker (1650) can be used by the camera navigation system to visually indicate to the user which sub-section the user is currently in with regards to the positioning of the cursor. Furthermore, in various embodiments, the ring associated with the cursor (410) can be make larger or smaller as the simulated laparoscope is moved closer or farther away, respectively. This could be used as an indicator for situations where the meter (1640) is not visible to gauge the simulated laparoscope's depth.

Furthermore, the size of the ring of the cursor (410), as provided by the camera navigation system, may affect how hard or easy it is to maintain the cursor (410) within the surrounding area (1620) of the moving target (1610). For example, a smaller ring may be easier to maintain within the surrounding area (1620) compared to a larger ring but the smaller ring would still need to be large enough to not intrude into the area denoted by the moving target (1610). Meanwhile a larger ring would have an easier time to move with the moving target (1610) and avoid intruding into the moving target's space but may have more difficulty staying within the surrounding area (1620). Thus, the challenge is to balance the distance from the moving target (1610) to have the size of the ring capable of being able to adapt to the moving target.

In various embodiments, the camera navigation system is configured to have the moving target (1610) wait until the cursor (410) is determined to be in an appropriate predefined location, orientation, and/or viewing distance. Visual indicators, such as those described above, can be provided by the camera navigation system to direct how to correct the cursor's position, orientation and/or viewing distance. Afterwards, the camera navigation system is configured to continue to move the moving target (1610) once an optimal view is achieved by the cursor (410) (corresponding to the desired location, orientation, and viewing distance). When the moving target (1610) is determined to have reached the end of the path (1630), the level ends and the exercise is deemed completed. Afterwards, the feedback related to the performance of the exercise is displayed.

Much like the feedback provided with the ‘trace’ exercise, the feedback provided by the camera navigation system for the ‘follow’ exercise may comprise of information used to quantify the user's and/or other users' performance. For example, the feedback may include graphs of rotation and viewing distance performance of the simulated laparoscope. Other feedback may include a bar graph showing how accurately the target was followed and the time in which the exercise was completed. Furthermore, the feedback may also include leaderboards showing previous scores of one user as well as scores from others who also completed the exercise. These scores may be organized in various different ways (i.e. ranked based on performance).

In various embodiments, the difficulty of this exercise as predefined or set by the camera navigation system can depend on a variety of factors. For example, the difficulty can be based on the complexity of the path, how fast the target is moving, the variation in speeds and/or stops performed by the target, the size of the target (i.e. ring), and allowable metrics associated with acceptable view obtained by the simulated laparoscope (e.g., how much of the simulated laparoscope's rotation is allowed, how much change in viewing distance is allowed), all or portions of which may be predetermined or selectable via the camera navigation system.

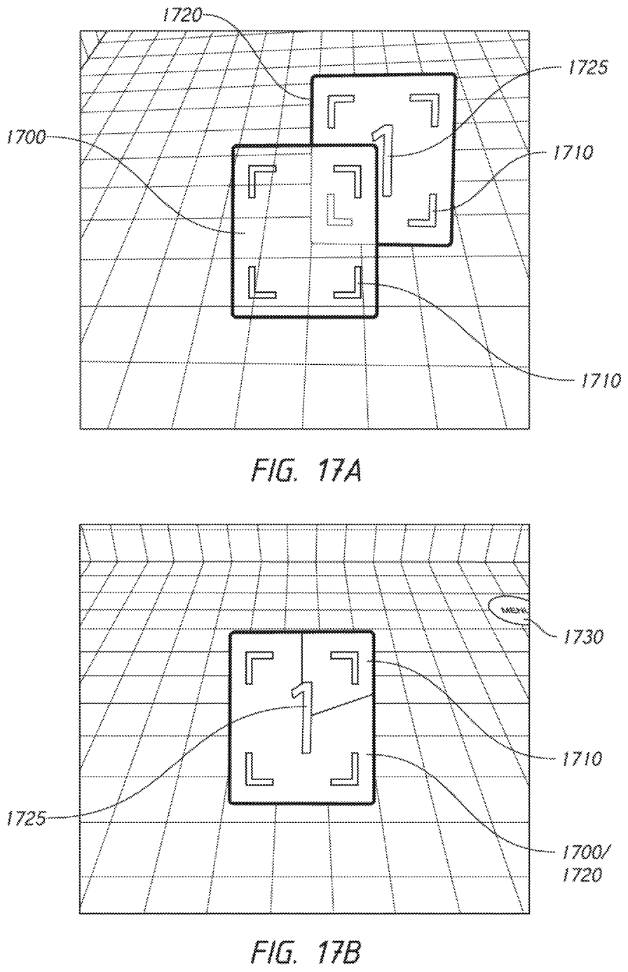

FIG. 17A and FIG. 17B illustrate an exemplary embodiment of a framing camera navigation exercise; another camera navigation exercise that the camera navigation system can provide. The ‘framing’ exercise measures and trains the ability to position and hold the cursor (1700) in a steady manner. The skill translates to an actual surgical procedure as surgical assistants are generally required to change and hold the position of laparoscopes in accordance with the surgeon's request.

During the ‘framing’ exercise one or more targets (1720) are presented by the camera navigation system. The targets (1720) may be placed in one or more random locations or at pre-determined locations within the digital environment (405). Furthermore, the targets (1720) may have a numerical identifier (1725) to help identify in what order the targets (1720) would need to be captured as well as how many targets (1720) there may be for the exercise. The targets (1720) will generally be facing towards the direction of the simulated laparoscope. As such, the arrangement of the targets (1720) within the digital environment for the ‘framing’ exercise is designed to simulate an operative site. A user (i.e. the surgeon's assistant) can then be instructed to maneuver the cursor to obtain the view of a desired location within the training environment. The camera navigation system determines whether the simulated laparoscope has properly framed the target by comparing the location of the cursor in the digital environment and comparing the with one or more targets assigned to the digital environment.

While the simulated laparoscope is being maneuvered relative to the training environment, the cursor (1700) will be displayed on the monitor. An embodiment can be seen in FIG. 17A which illustrates the cursor (1700) and one of the targets (1720). In various embodiments, the cursor (1700) corresponds to a desired view when the cursor is in the correct position with respect to one of the targets (1720). The cursor (1700), which can be transparent or partially translucent, is generated and remain at the center of the monitor. The goal is to maneuver the cursor (1700), such that the target (1720) is positioned in the same position as the cursor (1700) so that the cursor (1700) and the target (1720) overlap directly with each other as seen in FIG. 17B.

In various embodiments, the cursor (1700) and/or the target (1720) may include alignment markers (1710). The alignment markers (1710) may generally be represented by the camera navigation system as brackets internal to the cursor (1700 and/or the target (1720). The alignment markers (1710) provide further assistance in the ‘framing’ or overlapping of the cursor (1700) with the target (1720) by providing further reference points that users can rely on to determine how to move the cursor (1700) to overlap the target (1720).

In various embodiments, the camera navigation system is configured to determine that the simulated laparoscope (and in turn the cursor (1700) is held for a pre-determined period of time before moving to a different target (1720). In various embodiments, all the targets (1720) may be generated and shown at the start of the exercise. In other embodiments, subsequent targets (1720) may be provided by the camera navigation system only after successfully capturing a current target (1720) as determined by the camera navigation system.

The position of the cursor may not be the only factor in ensuring that the desired view is captured (corresponding to the overlap/matching of the overlay and the target). The simulated laparoscope positioning may also be another factor that is taken into account by the camera navigation system since any distortion caused by a different viewing angle may cause the cursor and the target to not completely line up. For example, if the simulated laparoscope has any roll, or if the simulated laparoscope is too close or too far away (i.e. viewing distance), the cursor and the target may not completely line up. In this way, the camera navigation system may store specific details regarding the correct location, orientation, and viewing distance for the cursor in the digital environment to be deemed properly acquiring a target (give or take a buffer threshold based on difficulty). It is the user's job to maneuver the simulated laparoscope in the way to achieve the corresponding location, orientation, and viewing distance for the cursor in order to “frame” the assigned target.

In various embodiments, when the cursor (1700) and the targets (1720) completely match or overlap a timer may be initiated by the scope view generator to track how long may be required to hold the position of the cursor and/or positioning of the simulated laparoscope. The timer may be illustrated or displayed by the camera navigation system as a bar that fills up from an empty state. Once full, the timer bar may indicate that the current target (1720) has been successfully ‘framed.’ In various embodiments, any motion that causes a mismatch of the cursor (1700) and the target (1720) may cause the timer bar to stop filling up, slowly empty, or completely empty. When the cursor (1700) and the target (1720) is again completely matched or overlapped, the timer bar can continue to fill up.

In various embodiments the ‘framing’ exercise is concluded after completing (i.e. matching/overlapping) a predetermined amount of targets (1720). The number of targets (1720) can vary based on the difficulty/level selected. Feedback, provided by the camera navigation system, in various embodiments, is also displayed in a similar manner as describes above for the earlier exercises provided by the camera navigation system. For example, the feedback for the ‘framing’ exercise provided by the camera navigation system can include a bar graph depicting how accurately the targets (1720) were lined up to the cursor (1700), the time it took to complete the exercise, and leaderboards showing previous scores of the user and/or other users who completed the same exercise. In various embodiments, the difficulty of the ‘framing’ exercise as set or predefined by the camera navigation system depends on the predefined tolerances that indicate whether the cards are matched or not, how long focus needs to be on each of the targets (1720), and/or how many targets (1720) need to be found.

In various embodiments, if the user wishes to exit the exercise prior to completion, a “home” button (1730) is provided. Interaction with the ‘home’ button (1730) with the overlay (1700), in various embodiments, is determined by the camera navigation system to allow the user to return back to the main menu screen or to select a different level or difficulty.

Although the some camera navigation exercises were described herein in the context of a zero-degree laparoscope, in various embodiments, an angled laparoscope is applicable, albeit with some calculations to convert the image data for an angled laparoscope to a corresponding zero-degree representation generated by the camera navigation system. In various embodiments, different exercises described next may be provided by the camera navigation system in connection with an ‘angled’ laparoscope. In various embodiments, these applications may not be compatible with a zero-degree laparoscope due to certain the physical limitations of the zero-degree laparoscope.

A difference between the zero-degree laparoscope and an angled (i.e. 30 degree) laparoscope is the arrangement of the image sensor/camera at the distal end of the simulated laparoscope. For the angled scope, the image sensor/camera is not aligned with the longitudinal axis of the simulated laparoscope. The angled arrangement adds complexity when and how the camera is rotated or otherwise manipulated with respect to the training environment.

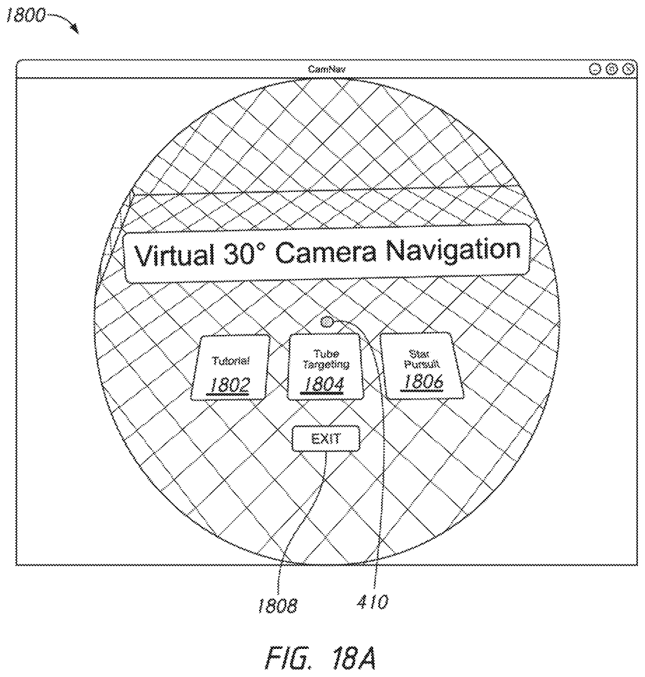

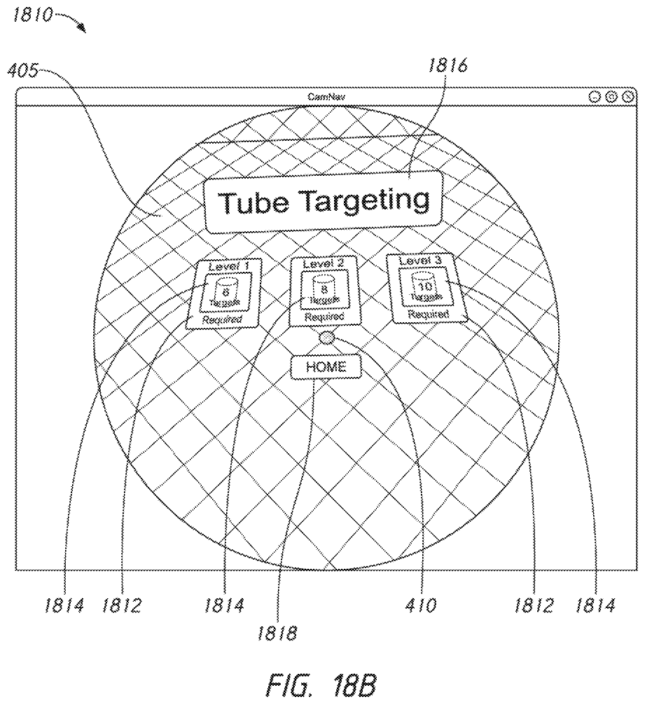

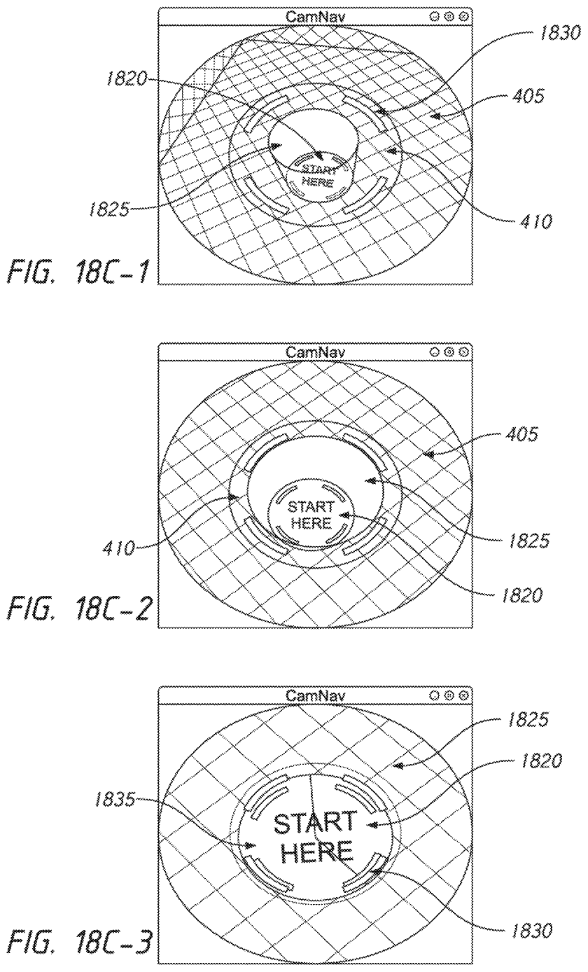

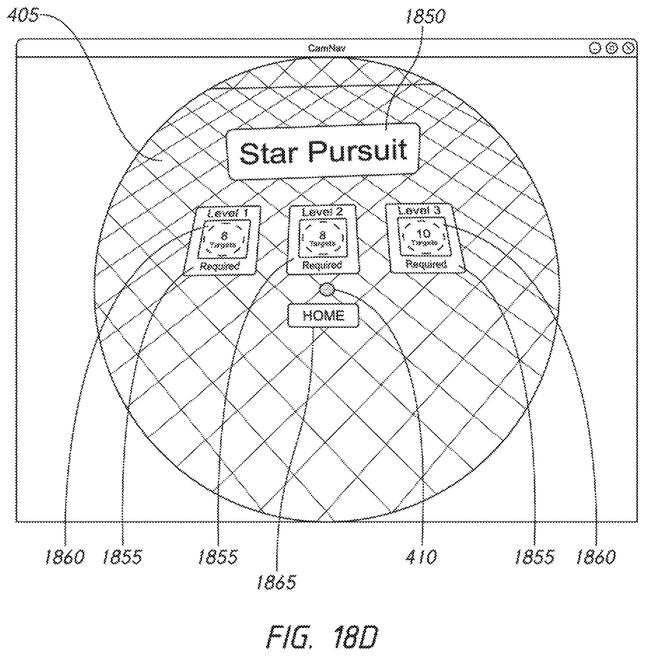

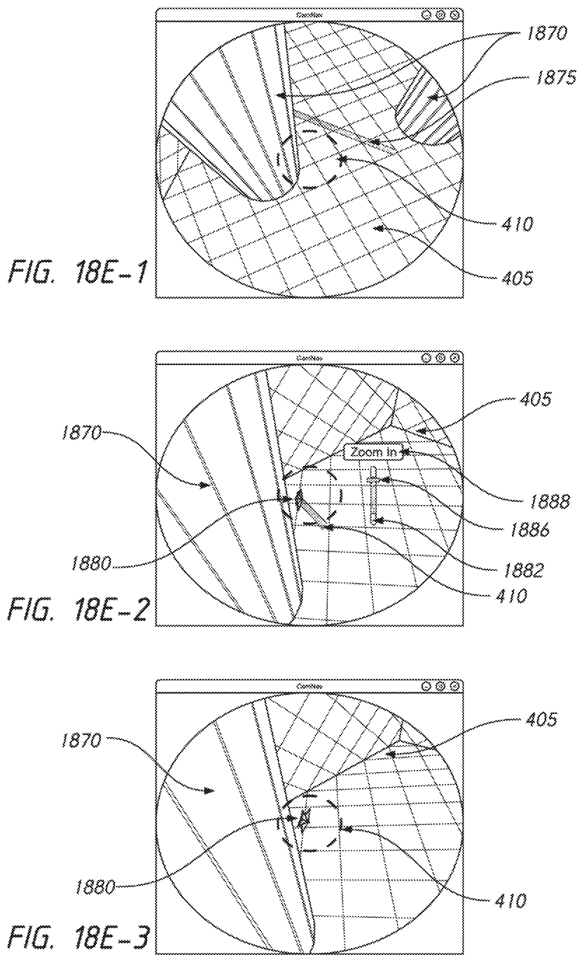

FIG. 18A-F IG. 18E-3 illustrate exemplary camera navigation exercises for a simulated angled laparoscope as provided by the camera navigation system. In particular, the figures illustrate at least two different camera navigation exercises: tube targeting and star pursuit.

As illustrated in FIG. 18A, a menu (1800) is provided that includes a tutorial option (1802) and exercises (1804, 1806) tailored for the ‘angled’ laparoscope. The tutorial option (1802) goes over how to maneuver the ‘angled’ laparoscope relative to the training environment and shows how those movements are translated to a cursor (405) within the digital environment (410). Due to the complexity of the angled laparoscope, the tutorial option (1802) as provided by the camera navigation system provides scenarios whereby users are able to use just one of the points of manipulation and see how changes in the angled laparoscope can change the view within the digital environment of where the image data will be captured.

In various embodiments, the camera navigation system performs the positional tracking of the simulated angled laparoscope with respect to the training environment different than the zero-degree laparoscope. That is because the user would need to not only rotate the camera capturing the image data but also further introduce additional rotation to modify the view which affects where the images are being captured. An explanation on the embodiment for the simulated angled laparoscope will be provided below. This explanation can be provided via the tutorial option (1802) so that the user is able to become more familiarized with the operation of the simulated angled scope.

In various embodiments, the simulated angled laparoscope has two points of manipulation that needs to be accounted for to properly identify the location within the training environment. The simulated angled laparoscope first of all operates differently by allowing users to “look around” objects or obstacles in order to view that would be otherwise obscured. To facilitate the maneuvering of the simulated angled laparoscope within the training environment, a first point of manipulation is with respect to rotation of the camera. In various embodiments, the rotation of the camera is performed at the distal end of the simulated angled laparoscope (i.e. a portion of the simulated angled laparoscope closet to a handle). A user manipulating the simulated angled laparoscope uses the rotation of the camera to ensure that the image being captured is oriented in the appropriate manner (e.g., right side up).

Different from the zero-degree laparoscope is the tracking of the physical rotation of the simulated laparoscope (i.e. the entire instrument). In various embodiments, a rotary sensor is provided with the simulated angled laparoscope in order to monitor and obtain the physical angle of rotation for the simulated angled laparoscope. In various embodiments, with the physical rotation of the angled portion of the simulated angled laparoscope, the camera is also rotated a corresponding amount. To maintain a steady image as the angled portion of the simulated angled laparoscope is being rotated, the camera would need to be rotated in the opposite direction by a corresponding amount to compensate for the rotation introduced. The rotation of the angled portion of the simulated angled laparoscope affects how the horizon is portrayed within the image data. In various embodiments, the horizon is desired to be arranged left to right in the middle of the image data. In various embodiments, the horizon is defined with a bottom of the camera or image sensor.

Unlike in the embodiment of a simulated zero-degree laparoscope, embodiments for the simulated angled laparoscope would not only utilize the image data of the markers captured by the camera but also an angle of rotation measures by a rotary sensor. Both sets of information would be used by the camera navigation system to determine the positional (6 degrees of freedom) information for the simulated angled laparoscope with respect to the training environment and how the cursor is displayed within the digital environment. Specifically the angular value obtained by the rotary sensor is added to the pitch value obtained about the simulated laparoscope via the PnP process. Furthermore, the digital environment would be generated in a way as to provide the specific point of view of the digital environment from the perspective of the cursor, for example, rotating the digital environment a corresponding amount based on the angle measured by the rotary sensor.

In various embodiments, the camera navigation system is configured to be suable with any number of different simulated laparoscopes. Though the camera navigation system may be provided with specific simulated laparoscopes (e.g., zero-degree and angled) that will be usable to participate in one or more camera navigation exercise, other (e.g., 3^{rd} party) simulated laparoscopes would also be compatible or usable with the camera navigation system described herein.

In various embodiments, the camera navigation system is configured to identify a type of simulated laparoscope being connected. In various embodiments, identification information for different simulated laparoscopes that can be used with the camera navigation system is stored in memory. In various embodiments, the camera navigation system is configured compare identifying information from the connected simulated laparoscope with the identification information stored in memory and determine if the connected simulated laparoscope is compatible. If compatibility is confirmed, in various embodiments, associated calibration information for the laparoscope is retrieved from memory that will be used to calibrate or otherwise transform the information being processed by the camera navigation to account for the various features specifically associated with the connected simulated laparoscope. For example, if the “field of view” of the connected simulated laparoscope is below a pre-determined threshold as determined by the camera navigation system, modifications may be performed by the camera navigation system during calculations of the position of the simulated laparoscope so that the processing provides a similar output as if the “field of view” is at the pre-determined threshold. In various embodiments, a corresponding set of camera navigation exercises that are compatible with the connected simulated laparoscope as determined by the camera navigation system will be retrieved from memory by the camera navigation system. The camera navigation system is configured to populate the digital environment with the compatible camera navigation exercises.

In embodiments where the connected simulated laparoscope is not recognized or identified as be compatible, such simulated laparoscopes would not be usable with the camera navigation system. However, a simulated laparoscope can be made to be compatible with the camera navigation system by way of calibration. In particular, based on the characteristics of a simulated laparoscope (e.g., field of view, zero degree/angled, quality of image), any unrecognized simulated laparoscope could undergo calibration with the camera navigation system in order to ensure that the information being captured by the unrecognized simulated laparoscope can be processed to be consistent with other recognized or compatible simulated laparoscopes. Such calibration, in various embodiments, may involve the camera navigation system changing or updating variables or adding different factors to align image information of the unrecognized simulated laparoscope with compatible simulated laparoscopes. The corresponding calibration data, in various embodiments, would be stored in memory alongside or associated with the identification information of the newly calibrated or recognized simulated laparoscope. Therefore, in future applications, the camera navigation system can recognize different simulated laparoscopes and allow them to be used with the various menus and camera navigation exercises.

FIG. 18A illustrates a menu that contains camera navigation exercises for the angled laparoscope. As seen in FIG. 18A, exemplary exercises for the simulated ‘angled’ laparoscope include a tube targeting exercise (1804) and a star pursuit exercise (1806). In various embodiments, additional camera navigation exercises (e.g., the zero-degree related camera navigation exercises discussed earlier) can also included in the menu (1800).

Each of the exercises (1804, 1806) described here are connected with the simulated ‘angled’ laparoscope, the camera navigation exercises as provided by the camera navigation system allow users the ability to practice, train and/or assess manipulations of the simulated laparoscope and the capturing of the image data using the ‘angled’ laparoscope; the camera navigation exercise would allow for users to enhance surgical or assisting surgical skills with the angled laparoscope.

In various embodiments, the capturing of image data from the insert or grid using an angled laparoscope may be more complicated than during the use of the zero-degree laparoscope because the orientation of the image sensor used to capture the image (housed within the ‘angled’ laparoscope) as well as the orientation of the plane the image is being captured resides in are adjustable and accountable. In various embodiments, control for the image sensor may be associated with one portion of the ‘angled’ laparoscope (e.g., handle or light cable) while control for the orientation of the plane of the image is associated with a different portion (e.g., camera head). In various embodiments, to properly capture an image using the simulated ‘angled’ laparoscope, a user would need to be adept in manipulating the two portions of the simulated ‘angled’ laparoscope to not only control the rotation of the image sensor but also maintain the orientation of with respect to the horizon.

With an example ‘tube targeting’ exercise, there are a pre-determined number of targets which may be positioned within the digital environment as provided by the camera navigation system. In various embodiments, the placement and/or positioning of the targets as provided by the camera navigation system would be the same for a difficulty level to maintain a consistent baseline which can be used to quantify (e.g., rank) subsequent performances by the user and/or performances by other users. However, each difficulty level may increase the number of targets to be captured as well as the complexity involved with maneuvering from one target to a subsequent target. A seen in FIG. 18B, an exemplary menu (1810) for the tube targeting exercise is shown. A title (1816) informs the user of the specific exercise that is currently being select.

As seen in the exemplary menu (1810), a number of different difficulty levels (1812) are provided within the digital environment (405) by the camera navigation system and selectable by a user using the cursor (410). The difficulty levels (1812) can have an associated level image (1814) which provides hints as to what the exercise entails; for example, a number of targets that would need to be acquired. User selection of one of the difficulty levels (1812) would require that the user maneuver the cursor (410) to overlap at least a portion of a specific difficulty level (1812) for a pre-determined period of time. Once selected, the camera navigation system will retrieve the related data from memory and update the digital environment (405) with the various targets and objects needed for the exercise.

In various embodiments, the different difficulty levels (1812) may be initially restricted or locked by the camera navigation system to prevent user access to them. Access is generally gained after fulfilling some prior criteria as determined and/or confirmed by the camera navigation system. For example, in order to access level 1, the user may be required to complete the tutorial; access to level 2 may require completion of level 1 with a pre-determined proficiency; and access to level 3 may require completion of level 2 with a pre-determined proficiency.

Each of the difficulty levels (1865) may show to the user how many targets (1867) will need to be acquired. The ability to select higher difficulties for the tube targeting may be based on completion of a previous level's difficulty and/or achieving a specific score/qualification.

Furthermore, the menu (1810) for the tube targeting exercise may also include a “home” button (1818). The “home” button (1818) will allow the user to exit the tube targeting menu (1810) and return to the main menu where the user could select the tutorial or any other available exercises.

With reference to FIG. 18C-1 to FIG. 18C-3, these figures illustrate an exemplary progression of the ‘tube targeting’ exercise is shown. With reference to these three figures, the figures illustrate the progression of the ‘tube targeting’ exercise. In particular, the simulated ‘angled’ laparoscope is manipulated with reference to the training environment so that the cursor (410) displayed within the digital environment (405) is aligned with one of the targets (1820) displayed therein. The alignment of the cursor (410) with the target (1820) is achieved when the cursor (410) (comprising a designator, e.g., a greyed area (e.g., circle) with markings (1830) highlighting an edge or outer perimeter of the greyed area), is determined to have interacted/ overlaps with one of the targets (1820) displayed within the digital environment. Although the figures illustrate (and the present description describes) one type of cursor (410), other types of cursor (410) having different shapes are also compatible and usable with the exercise.

To be able to maneuver the cursor (410) around the digital environment (405) using the angled laparoscope, the user would need to become adept at manipulating the angled laparoscope using both points of manipulation for the simulated angled laparoscope. To maintain a correct horizon, the user would need to utilize both points of manipulation as the use of only one of the two may cause the image being captured to rotate (e.g., become upside-down). Since the user is not merely placing the simulated laparoscope overhead the target (1820), the user is instead tested of their expertise of controlling the angled laparoscope through the use of the first and second points of manipulation for the angled laparoscope. In particular, changes in the perspective view of the cursor within the digital environment requires the user to view (or otherwise acquire) the target (1825) without the perspective colliding with the tube (1825) or other nearby obstacles within the digital environment (405). Furthermore, both points of manipulation would need to be used, otherwise the image data captured by the angled laparoscope would appear rotated by some amount and not maintain its right side up orientation.

As a camera navigation exercise is carried out, a user may be instructed by the camera navigation system to highlight a number of targets (1820) within the digital environment (405) using the cursor (410). While one or more of the targets (1820) may not have any obstacles (or portions of a tube (1825) used to obscure alignment of the cursor (410) with the target (1820), tubes (1825) may also be present which would otherwise obscure at least a portion of the target (1820) from any viewing direction (i.e. point of view of a user) except directly overhead. As seen in FIG. 18C-1, an exemplary view as provided by the camera navigation system illustrates a view via the ‘angled’ laparoscope with a target (1820) positioned within a tube (1825). The walls of the tube (1825) may be partially transparent to allow viewing and/or displaying of the target (1820) within the tube (1825). However, proper acquisition of the target (1820) using the cursor (410) is only achieved or determined to be achieved by the camera navigation system when the cursor (410) is identified by the camera navigation system to encompass the entirety of the target (1820) without interference from the walls of the tube (1825). Even if the target (1820) is encompassed within the cursor (410), as illustrated in FIG. 18C-2, if portions of the walls of the tube (1825) are also found within the cursor (410), this may not desired (for example based on the difficulty of the exercise as predefined by the camera navigation exercise) and thus would not correspond to an appropriate acquisition of the target (1820) as determined by the camera navigation system. With reference to FIG. 18C-3, the cursor (410) is shown to have encompassed the entirety of the target (1880) without portions of the tube (1825) therein. In particular, portions of the tube (1825), though still visible are positioned outside of the cursor (410). In various embodiments, proper acquisition may be achieved or determined to be achieved by the camera navigation system when only the entirety of the target (1820) is within the boundaries set by the markings (1830) of the cursor (410).

Determination regarding whether the cursor (410) has properly encompassed the target (1820) can be done by storing, with the exercise, specific details regarding the target (1820) and where the cursor (410) would need to be located (e.g., location, depth, perspective) to properly acquire the target (1820). As referred to herein, the cursor's location corresponds to an x, y, and z set of coordinates that highlights where in the three-dimensional space the cursor should be found. The viewing distance is a representation of how far away the simulated laparoscope is from the training environment. The viewing distance can be illustrated by having the cursor change in size accordingly. For example, if the simulated laparoscope is close to the training environment, the cursor may be made larger. In another embodiment, the viewing distance can represent when the simulated laparoscope is held farther away from the training environment. The viewing distance can be reflected accordingly with a smaller size of the cursor. Cursor perspective pertains to the direction and resulting rotation being simulated; in particular, the field of view is a simulated perspective of the digital environment that corresponds to the positional information from the simulated laparoscope. In various embodiments, the viewing distance can also be reflected by having the digital environment (i.e. the perspective of the digital environment from the point of the cursor) become bigger or smaller based on how close or far away the simulated laparoscope is from the training environment (i.e. the viewing distance).

If the camera navigation system detects and determines that the cursor (410) has a location, depth, and/or perspective near the values assigned with a particular target (1820), it can be concluded that the target (1820) was properly encompassed by the cursor (410). In various embodiments, in addition with the comparison with the stored details regarding the target (1820) and the cursor (410), collision calculations can also be performed between the cursor (410) and the location of the tube (1825) to ensure that no collisions are detected which would indicate that the target is at least partially obscured.

Once the cursor (410) is appropriately placed over the target (1820) and identified by the camera navigation system, the camera navigation system can generate and provide a notification to the user. As illustrated in FIG. 18C-3, the camera navigation system may begin to fill the cursor (410) in a clockwise direction (e.g., changing from a first color or shading to a second color or shading) to identify and inform a user that the cursor is in an appropriate position with respect to the target. In various embodiments, the camera navigation system provides the cursor (410) to be used as a meter or gauge (1835) to highlight not only that the target (1820) was properly acquired but for how long the target (1820) should be maintained within the cursor (410). The rate at which the meter (1835) changes, e.g., fills and/or color changes, can correspond to an amount of time required to hold the simulated ‘angled’ laparoscope at the designated position. Other ways of notifying the user, via other visual notices (e.g., outline of the cursor (410) changes color) or audio notices (e.g., beeps) when the cursor (410) properly interacts with the target (1820).

Once a target (1820) has been acquired for a pre-determined amount of time as determined by the camera navigation system, the next target (1820) can be displayed. The previously captured target (1820) and tube (1825) can be removed from the digital environment (405) by the camera navigation system to reduce or eliminate confusion regarding which target (1820) should be acquired next or later on during the exercise. After all the pre-determined targets (1820) have been acquired, the camera navigation system can conclude the exercise. In various embodiments, feedback regarding the user's performance can then be provided, e.g., metrics of the user's performance of the exercises (e.g., time of complete, #of collisions, accuracy, steadiness).

In various embodiments, another exercise provided by the camera navigation system and used in connection with the ‘angled’ scope is called ‘star pursuit.’ In this exercise, the user is tasked with following and locating an object (e.g., star-shaped target) that moves around the digital environment stopping at pre-determined spots. As with other exercises, there may a menu (1860) for the star pursuit exercise where users would be able to select from different difficulty levels (1855) as seen in FIG. 18D. Each of the difficulty levels (1855) may have an image therein (1860) which provides information about that level, for example, identifying how many targets would need to be acquired. Access to the different levels may initially be restricted but subsequently granted so long as the user fulfills the associated requirements such as completing the tutorial and/or completing the lower difficulty levels.

User selection of the levels is performed in a similar manner as discussed above for the tube targeting by using the cursor (410) within the digital environment (405). The star pursuit menu (1850) also includes its own “home” button 1865 which allows the user to return to the main menu.

With reference to FIG. 18E-1 to FIG. 18E-3, an example progression of the “star pursuit” exercise is shown. During the exercise, one or more objects (e.g., tubes/pillars) (1870) may be placed within the digital environment (405) designed to obscure a direct view from the cursor (410) to the target (1880). In this embodiment, the target (1880) is star-shaped, however other different shapes are possible. The placement and number of objects (1870), depending on the difficulty level, are provided by the camera navigation system to make acquiring the moving target (1880) more difficult. The camera navigation system provides a cursor (e.g., a circle with an outline) (410) which defines how the moving target (1880) should be properly acquired. In various embodiments, the user is instructed to move the cursor (410) towards the target (1880) and ensure that the target (1880) is encircled by the cursor (410).

As mentioned above, movements within the three-dimensional space of the digital environment requires the user to manipulate the two points of manipulation for the simulated ‘angled’ laparoscope to not only maintain a horizon (i.e. the image being in an upright orientation) but also be able change the perspective and what is being viewed within the digital environment (405). If only one of the two points of manipulation is used, the image being captured by the camera/image sensor would be rotated some amount. When the target (1880) is within the cursor (410) as determined by the camera navigation system, notifications can be provided to indicate whether the target (1880) is properly situated within the cursor (410). For example, the cursor (410) may change colors (as seen in FIG. 18E-3). If the cursor (410) is not properly positioned, e.g., needs to be adjusted (e.g., moved further or closer, rotated to the proper plane), an indicator or meter (1882) can be provided by the camera navigation system as seen in FIG. 18E-2.

The meter (1882) can be used by the camera navigation system to quantity the user's performance using multiple different zones. The meter (1882) has a marker (1886) which indicates the user's performance based on the different zones. Furthermore, the meter (1882) can also have hints or remarks (1888) that the camera navigation system can provide information to the user on how the simulated ‘angled’ laparoscope would need to be moved to better acquire the moving target (1880).

Once the target (1880) has been properly acquired as determined by the camera navigation system, in some embodiments for a pre-determined period of time, the moving target (1880) can then be moved to a next position within the digital environment (405). The moving of the target (1880) challenges the user to maneuver the simulated ‘angled’ laparoscope to the new location while navigating around one or more objects (1870). In various embodiments, hints can be included to assist the user in locating where the target (1880) moved to. For example, an arrow can be used to highlight where the target moved to. In some embodiments, a line or trail (1875) can be left by the target (1880) as it moves to its next location (as seen in FIG. 18E-1). The line or trail (1875) may be visible for a pre-determined amount of time or until the target (1880) has been acquired.

After the moving target (1880) has been acquired a pre-determined number of times as determined by the camera navigation system, the star pursuit exercise can conclude. In various embodiments, feedback regarding the user's performance can then be provided which quantifies the user's performance of the exercises (e.g., time of complete, #of collisions with objects, accuracy, and/or steadiness).

In connection with both of the exercises (‘tube targeting’ and ‘star pursuit’) used with the simulated ‘angled’ laparoscope, implementation of collision detection by the camera navigation system is used to quantify whether the target has been properly acquired. For example, for the ‘tube targeting,’ the camera navigation system performs collision detection checks to see if the user's view of the target (corresponding to the perspective of the cursor) is directly overhead of the target and not otherwise being obscured by the various tubes in the digital environment. It may be possible to view a target within a tube if the cursor is at an angle with the target or if the tube is transparent. However, in various embodiments, since the tube does not physically exist, such instances of acquisition of targets are restricted or prevented by the camera navigation system and instead only acquisition is only determined by the camera navigation system when the cursor is directly overhead. In various embodiments, the collision detection is configured to identify instances when at least a portion of the tube is between where the target is located and the cursor's perspective. In various embodiments, the collision detection simulates target targeting where physical object obstruct a user's view and encourages users to maneuver to a more appropriate location (i.e. overhead). In various embodiments, a pre-determined amount of obstruction (calculated using the collision detection) via objects (i.e. tubes) may be permitted which may vary due to the difficulty of a selected exercise.

In various embodiments, since the location of the target and objects are known (i.e. stored in memory associated with a selected exercise), the camera navigation system performs collision detection by calculating a path from the current location of the cursor to the target and identifying if there are any points along that path that intersect with known location of tubes or other obstacles. If at least one point on the path intersects with a location of a tube or obstacle, a collision is detected by the camera navigation system which would correspond to at least a portion of the view between the cursor and the target being obstructed by the tube. In various embodiments, based on the difficulty of the exercise being performed, some amount of collision below a threshold may be allowable by the camera navigation system.

With the ‘star pursuit’ exercise, the collision detection is used to determine if the user's view of the star-shaped target is unobstructed (within a defined acceptable degree) by the various objects (e.g., tubes) within the digital environment. In the same manner as in the tube targeting, the camera navigation system checks known location of the various objects between a path of the cursor and the star-shaped object. If at least one object is located on the path, this can be indicative that the view is at least partially obstructed. In various embodiments, the user may be encouraged to maneuver the simulated ‘angled’ laparoscope to a different angle or positioning to properly “look around” the object or to pursue a different location and/or orientation to at least obtain a different view of the star-shaped target that is not obscured by an object (e.g., tube).

As noted above, feedback is provided by the camera navigation system after the exercises associated with the simulated angled laparoscope are completed. An evaluation of the user's and/or users' performance is provided, for example, via a score that is calculated on various criteria such as being based on the amount of time it took to complete the exercise. Other criteria are also possible and contemplated as appropriate to characterize and highlight areas of improvement for the user.

With each of the different exercises provided by the camera navigation system, how the feedback is obtained by the camera navigation system from performance data determined by the camera navigation system to characterize a performance during the exercise can be different and customized accordingly. For example, in various embodiments, an emphasis or greater weight can be provided for feedback related towards simulated laparoscope rotation and viewing distance, as these skills have a more significant impact on the quality of camera navigation during an actual surgical procedure. In other embodiments, speed may be more significant; though speed can also be provided a lesser importance. While completing an exercise quickly is a sign of mastery, completion of the exercise should not be attained at the expense of lower scores in other skills such as roll, viewing distance, and accuracy. In various embodiments, these criteria can be predefined by the camera navigation system and/or adjusted by the camera navigation system based on a user's prior experiences and/or other criteria, e.g., predefined or set by an instructor and/or evaluator. For example, the criteria for practicing surgeons can be different from a set of criteria for students.

In various embodiments, the feedback discussed above for the different exercises can be is implemented in leader boards (see FIG. 9). The leader boards display the feedback (i.e. scores) of a particular user as well as the feedback (i.e. scores) from various different users. How the user's different performances of the same exercise or perhaps the performances of a group of users are ranked may be based on a specific order or weight of the factors. For example, in one embodiment, the leader boards may weight having performance of the camera's rotation higher than performance of the viewing distance scores. Other factors could subsequently be used (with decreasing significance) in ranking such as accuracy and time. The different scores can be saved locally, for example, to a database so that users data can be tracked and their progress over time can be retrieved for feedback without an instructor being present. In various embodiments, the performance data and feedback can be stored remotely (e.g., in the cloud) so that users in different physical locations can compare their performance with others from all over.

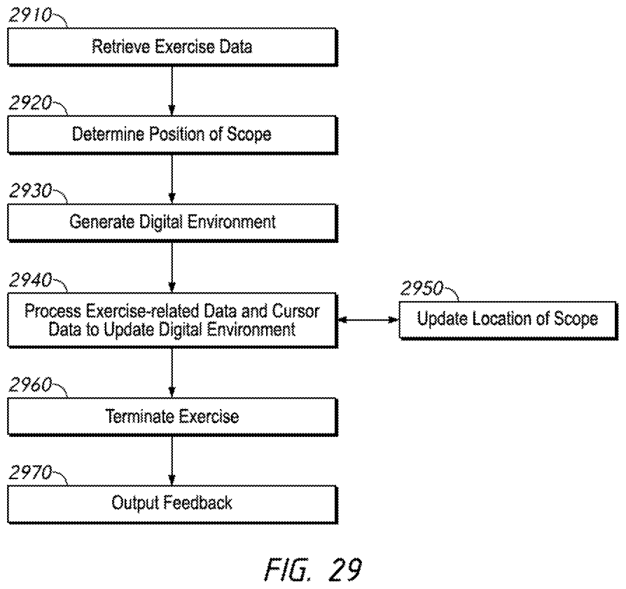

FIG. 29 illustrates an exemplary embodiment of the camera navigation system. Specifically, the figure shows an example flowchart outlining steps or operations that the camera navigation system may undergo in connection with any one of the exercises used with training associated with the simulated laparoscope. It should be noted that the figure provides a general overview of the steps or operations and that more or less steps or operations may be used, their order varied, and/or executed serially and/or in parallel, as appropriate.

Once the user has connected and inserted a simulated laparoscope (e.g., zero degree or angled), that the user would like to practice with, a corresponding set of exercises can be identified and shown by the camera navigation system via the menu (e.g., FIG. 4A or FIG. 18A) (2910). The user can then select one of the exercises and a corresponding difficulty. The camera navigation system retrieves the associated data for the digital environment and features (e.g., targets, obstacles, paths) that will need to be rendered for the exercise.

Once the exercise begins, data is obtained from the simulated laparoscope from within the training environment. In various embodiments, image data is obtained from the simulated laparoscope of the training environment. The computer vision portion of the camera navigation system (e. g, the scope view generator) then processes the markers captured in the image data (via computer vision processes) to determine information about the position of the simulated laparoscope with reference to the training environment (2920).

A digital environment corresponding to the selected exercise is generated (2930) by the camera navigation system. Depending on the exercise being performed, the corresponding features are retrieved from memory and generated within the digital environment, for example, markers, obstacles, paths. Using the positional information previously obtained for the simulated laparoscope, a corresponding perspective of the digital environment is provided. This corresponding perspective (e.g., view of the digital environment from the perspective of the laparoscope) is generated by the scope view generator and displayed on the monitor for a user to view. In various embodiments, the size/dimensions of the digital environment is a 1-to-1 correspondence with the training environment.

Once the position of the simulated laparoscope from the training environment has been conveyed within the digital environment as a cursor, in various embodiments, the camera navigation system (e.g., the scope view generator) then processes the data associated with the selected exercise with the location of the cursor in the digital environment. In various embodiments, the camera navigation system's exercises utilize information from the digital environment and stored information related to the selected exercise. These system may determine whether a particular path is being followed, is colliding with various obstacles, or properly acquiring a target. The system use the stored information regarding the features of the digital environment (e.g., path, marker, obstacles) with the position information of the simulated laparoscope within the digital environment. The camera navigation system is configured to provide different exercises and difficulties with different sets of information to use. For example, if the position of the cursor has the same location information (e.g., coordinates) as a target, this may be used by the camera navigation system to determine and/or indicate that the target has been acquired. In another example, if the position of the cursor is within a pre-determined ranged of locations, this may be used by the camera navigation system to determine and/or indicate that the target is within a pre-defined path.

At pre-determined intervals, the location of the cursor may be updated by monitoring/tracking any updates to the position of the simulated laparoscope with respect to the training environment (2950). For example, in various embodiments, the location may be updated every second (e.g., 60 times a second). The updated location for the simulated laparoscope can then be processed and transferred to the digital environment (2940). Any additional subsequent processing associated with the exercise being performed can then be performed with the stored information associated with the exercise (e.g., locations of targets, objects). For example, updates on the user's progress along a path or determining if a target has yet been acquired will compare and use the cursor's location within the digital environment and the stored information of the relevant features (e.g., path, markers, obstacles) for the exercise being performed. In various embodiments, a pre-determined set of coordinates for a location for the cursor may be stored for each exercise corresponding to the location where the cursor may need to be in order to properly acquire a target (which takes into account the framing aspect of the target being at the proper location, orientation, and depth). Thus, a comparison of the cursor's current position and the stored location can be used to determine if the target is properly framed or acquired by the cursor.

The aforementioned updating and processing for the simulated laparoscope's position in accordance with various embodiments can be continued by the camera navigation system for as long as the exercise is being performed. Once the user has completed the exercise, the system can automatically terminate the exercise (2960). In various embodiments, users may be provided by the camera navigation system the option to repeat the exercise or select a different exercise.

Once terminated, the camera navigation system can provide related feedback to the user based on the user's performance during the exercise (2970). The feedback is based on data acquired during the exercise within the digital environment. In various embodiments, for example, tracking accuracy of the user's performance can be based on an number of times the simulated laparoscope's position was determined to be within the pre-defined locations associated with the path. In various embodiments, for example, collision can be tracked based on how often the simulated laparoscope's position correspond to location of one or more objects within the digital environment.

In various embodiments, the specialized applications provided and/or associated with the camera navigation system that provides for the camera navigation exercises can be written in any number of computing languages (e.g., C+, C++) so that the applications can run natively on any combination of processor, computing device, and/or operating system desired. Furthermore, the application can also be written using web-based language (e.g., WebAssembly) such that the application can be run off browsers or the cloud/web.

For web-based applications, access and control of related hardware (such as the camera, rotary encoder, GPU, etc. . . . ) can be controlled through a web-browser. Specific application(s)/program(s) can be written and provided with the camera navigation system so that it is able to use APIs or other routines to access the related hardware. Data, along with the access to the hardware, is then passed on to web-based application so that it generates and renders corresponding user interface and images that will be displayed on the monitor for viewing and interactions. In accordance with various embodiments, the above described exercises, user interfaces, feedback, elements, images, metrics, and/or the like are provided by the camera navigation system and in various embodiments, via one or more local or remote processors and/or computing devices of the camera navigation system and/or scope view generator (and in various embodiments, via applications, programs, and/or libraries) configured to provide, generate, display, update, operate and/or evaluate the above exercises, user interfaces, feedback, elements, images, metrics, and/or the like as well as monitor and/or track a simulated laparoscope and/or evaluate data, interaction and/or generate interaction with an insert or grid, a surgical environment and a simulated laparoscope.

As mentioned above, the camera navigation system may include a surgical trainer and/or a body form associated with the training environment. In various embodiments, the body form may be a surgical trainer that is configured to simulate a torso of a patient. The body form or surgical trainer, in various embodiments, is configured to receive the insert or grid (as discussed above) to simulate conditions associated with laparoscopic surgical procedures performed within a patient. FIG. 30 illustrates an exemplary surgical trainer. With reference to the figure, the surgical trainer 3010 is illustrated as an exemplary embodiment having an top perspective view.

The surgical trainer 3010, as illustrated in the figure, is configured to simulate conditions associated with laparoscopic procedures such as the torso, abdominal, pelvic and/or other regions of a patient. One feature that facilitates the simulation of the conditions associated with laparoscopic procedures is that the surgical trainer 3010 can be set up to obscure direct vision of the insert or grid being practiced on that is housed within the surgical trainer 3010.

With continued reference to FIG. 30, the surgical trainer 3010 provides a body cavity 3012 that is substantially obscured from direct view. The body cavity is configured to receive the insert or grid or the like described in this invention. In some embodiments, the body cavity 3012 is accessible via a tissue simulation region 3014 that is penetrated by surgical instruments (e.g., laparoscopic devices) for the purposes of practicing surgical techniques (i.e. interacting with the insert or grid) located in the body cavity 3012. In various embodiments, the body cavity 3012 can also be accessible through a hand-assisted access device or single-site port device that is alternatively employed to access the body cavity 3012. In various embodiments, the body cavity 3012 can be accessible via both the tissue simulation region 3014 and the hand-assisted access device or single-site port device. In various embodiments, the body cavity 3012 is accessible via adapters, apertures or the like attached or integrated with the surgical trainer. An exemplary surgical training device is described in U.S. patent application Ser. No. 13/248,449 entitled “Portable Laparoscopic Trainer” filed on Sep. 29, 2011 and incorporated herein by reference in its entirety.

To obscure the body cavity 3012 from direct vision, the surgical trainer 3010 is designed to have a top cover 3016 that is connected to and spaced apart from a base 3020 via at least one leg 3020. In various embodiments, the surgical trainer 3010 may have more than one leg 3020. With the top cover 16, the base 3020, and the at least one leg 3020, the surgical trainer 3010 is configured to simulate laparoscopic conditions whereby the body cavity 3012 is obscured from direct vision. Such laparoscopic conditions may correspond to procedures that pertain to a surgeon operating on tissues or organs that reside in an interior of a patient (e.g., body cavity) such as the abdominal region. Thus, the surgical trainer 3010 is a useful tool for teaching, practicing, and demonstrating surgical procedures with their related surgical instruments by simulating a patient undergoing the surgical procedures.

As described above, the surgical instruments are inserted into the body cavity 3012 through one or more tissue simulation regions 3014 as well as through pre-established apertures 3022 via hand-assisted access devices or single-site port devices located in the top cover 3016 of the surgical trainer 3010. Although openings may be pre-formed in the top cover 3016, various surgical instruments and techniques can also be used to penetrate the top cover 3016 in order to access the body cavity 3012 thereby allowing for further simulation of surgical procedures. Once inside the body cavity 3012, interaction with the insert or grid using the simulated laparoscope/camera is possible; the insert or grid being located in the body cavity 3012 between the top cover 3016 and the base 3018.

With continued reference to FIG. 30, in various embodiments, the insert or grid may be a separate component but is secured beneath one or more of the tissue simulation region 3014 or apertures 3022 located in the top cover to ensure that the insert or grid does not move while the surgical trainer 3010 is in use. To secure the insert or grid housed in the body cavity 3012, the base 3018 may be designed to have a receiving area 3024 or tray that is configured to stage or secure the insert or grid in place within the surgical trainer 3010. In various embodiments, the receiving area 3024 of the base 3018 may include attachment elements for holding the insert or grid in place. The attachment elements would interface with at least a part of the insert or grid and prevent the insert or grid from moving or shifting around while the surgical trainer 3010 was in use. In various embodiments, the insert or grid is removable and interchangeable with other inserts or grids as the attachment elements are configured to accept multiple different inserts or grids.

Other means for securing the insert or grid within the body cavity 3012 are also contemplated. For example, the insert or grid may be secured to the base 3018 via the use of a patch of hook-and-loop type fastening material such as VELCRO® which allows for the insert or grid to be removably connected to the base 3018. Other embodiments may utilize other attachment methods which may not provide removable connectivity between the base 3018 and the insert or grid. For example, adhesives can also be used to provide more connections between the base 3018 and the insert or grid that are not easily removable.

In various embodiments, a video display monitor 3028 is provided with the surgical trainer 3010. For example, the video display monitor 3028 can be hinged to the top cover 3016 and have at least two different states: a closed state where the video display monitor 3028 is hidden and an open state where the video display monitor 3028 can be viewed. In various embodiments the video display monitor 3028 can be separate from the top cover 3016 but still communicatively connected with the surgical trainer 3010.

In various embodiments, the video display monitor 3028 is communicatively connected to a variety of visual systems that deliver an image to the video display monitor 3028. For example, a laparoscope inserted through one of the pre-established apertures 3022 or an image capturing device (e.g., webcam) located in the body cavity 3012 can be configured to capture images of the simulated procedure being performed and transfer the captured images back to the video display monitor 3028 and/or other computing devices (e.g., desktop, mobile device) so that the images can be viewed regarding the area within the surgical trainer 3010. In various embodiments, other devices (e.g., microphones, sensors) may also be usable with the surgical trainer 3010 in order to capture other types of data such as audio data which can be combined with the visual data and displayed on the video display monitor 3028.

The surgical trainer 3010 can be configured to receive portable memory storage devices such as flash drives, smart phones, digital audio or video players, or other digital mobile devices that further facilitate in the recording of the simulated surgical procedure and/or playback of the data obtained from the surgical trainer 3010 onto a monitor for demonstration purposes. In various embodiments, additional or alternative (e.g., larger) audio visual devices can be connected to the surgical trainer 3010 that are usable to display the audio-visual data obtained from the surgical training device 3010. In various embodiments, the surgical trainer 3010 may be communicatively connected (e.g., wired or wireless) to a different computing device (e.g., desktop, laptop, mobile device) which is configured to receive data obtained from the surgical training device 3010 and display that data for others to view. Such embodiments may be useful in variations of the surgical trainer 3010 which do not include the video display monitor 3028.

As illustrated in FIG. 30, the top cover 3016 is generally positioned directly over the base 3018 with the one or more legs 3020 located substantially around the periphery. The legs 3020 interconnect between the top cover 3016 and base 3018. In embodiments where there are two or more legs 3020, each of the legs may be spaced apart equidistance from each other and act as a structural support holding the top cover 3016 in place above the base 3018. In various embodiments, the top cover 3016 and the base 3018 are substantially the same shape and size and have substantially the same peripheral outline. In various embodiments, the shape may correspond to the shape of the human anatomy such as the torso/abdominal region of a patient.

Depending on the arrangement of the top cover 3016, base 3018, and the one or more legs 3020, in various embodiments, the body cavity 3012 may be partially or entirely obscured from direct view. In some variations, the legs 3020 may include openings to allow ambient light to illuminate the body cavity 3012 as well as provide weight reduction for the overall surgical trainer 3010. Apertures associated with the legs 3020 may also allow vision and/or access into the body cavity 3012 of the surgical training device 3010.

In various embodiments, the top cover 3016 is removable from the one or more legs 3020. In addition, in various embodiments, each of the legs are removable or collapsible with respect to the base 3018. These features allow conversion of the surgical trainer 3010 into a portable form which has a reduced height.

In various embodiments, a camera navigation system is provided. The camera navigation system comprises a training environment; a simulated laparoscope configured to capture one or more images from the training environment; a scope view generator that determines positional information of the simulated laparoscope with respect to the training environment from the captured one or more images and generates a digital environment; and a monitor that is configured to receive and display the digital environment from the scope view generator.

In various embodiments, the camera navigation system is also configured to generate computer-generated elements to incorporate with the digital environment. The computer-generated elements configured for menus and/or camera navigation exercises.

In various embodiments, the camera navigation system is also configured to generate supplemental graphics elements, and the augmented image data generated by the scope view generator uses the generated supplemental graphics elements to be superimposed on at least part of the captured image data from the simulated laparoscope.

In various embodiments, the camera navigation system also includes a surgical trainer configured to simulate a torso of a patient, the surgical trainer having a top cover that is spaced apart from a base that defines the internal cavity.

In various embodiments, the internal cavity of the training environment also includes an insert or grid, the insert or grid comprising a plurality of markers arranged on a flat or planar sheet.

In various embodiments, the insert or grid is positioned on the base of the surgical trainer. In various embodiments, the insert or grid is positioned on the ceiling and/or one or more of the side walls including front and/or back walls of the surgical trainer. In various embodiments, the insert or grid is positioned on one or more objects or obstacles positioned within the internal cavity of the surgical trainer.

In various embodiments, the digital environment corresponds or comprises one or more simulated surgical exercises. In various embodiments, the one or more simulated surgical exercises comprises a follow exercise whereby the simulated laparoscope is directed to following a path; a track exercise whereby the simulated laparoscope is directed to follow a moving target; and/or a framing exercise whereby the simulated laparoscope is directed to overlap one or more targets.

In various embodiments, the scope view generator further monitors movement of the simulated laparoscope within the training environment and updates the digital environment to include a cursor, wherein a position of the cursor on the monitor corresponds to the position of the simulated laparoscope relative to the training environment.

In various embodiments, the scope view generator further monitors the position of the simulated laparoscope relative to the training environment, determines that the position of the cursor overlaps at least one computer-generated element associated with the digital environment, and updates the digital environment with different computer-generated elements based on the at least one computer-generated element that was overlapped by the cursor.

In various embodiments, the determination of the position and orientation of the simulated laparoscope within the training environment comprises identifying two or more adjacent markers found within the captured one or more images.

In various embodiments, the scope view generator further evaluates performance of the one or more simulated surgical exercises, and generates feedback based on the evaluated performance. In various embodiments, the generated feedback includes generating a leaderboard that includes evaluated performances of a plurality of different users. In various embodiments, the generated feedback includes visual indicators providing directions to complete the one or more simulated surgical exercises.

In various embodiments, wherein part or all of the scope view generator is implemented remotely from the training environment, wherein remote implementation comprises the scope view generator being run on a cloud-based server or a remote server.

In various embodiments, the simulated laparoscope simulates a 0-degree laparoscope or an angled laparoscope. In various embodiments, a camera navigation system comprises a plurality of markers and a scope view generator configured to use a subset of the plurality of markers to determine a position of a simulated laparoscope. In various embodiments, a system, e.g., a camera navigation system, comprises a plurality of markers and a view generator, e.g., a scope view generator, configured to use the plurality of markers to generate a digital environment. In various embodiments, a camera navigation system comprises a scope view generator configured to determine a position of a simulated laparoscope and/or to generate a digital environment utilizing image data captured by a simulated laparoscope. In various embodiments, a system, e.g., a camera navigation system, comprises a view generator, e.g., a scope view generator, configured to receive and/or retrieve image data and to determine a position of a device, e.g., a simulated laparoscope, and/or to generate a digital environment utilizing the image data.

In various embodiments, a camera navigation system is provided. The camera navigation system comprises a plurality of markers positioned on one or more planar surfaces; a

1. A camera navigation system comprising:
a surgical trainer;
a camera navigation box removably housed within the surgical trainer, the camera navigation box comprising a first side wall, a second side wall, a third side wall, and a base positioned between the first side wall, the second side wall, and the third side wall;
a simulated laparoscope movable through a top cover of the surgical trainer, into the camera navigation box, and within the camera navigation box;
a training environment comprising a first insert, a second insert, a third insert, and a fourth insert, each insert of the first, second, third, and fourth inserts being planar sheets and comprising a plurality of markers configured to be used to track a position of the simulated laparoscope, the first insert positioned on the first side wall of the camera navigation box, the second insert positioned on the second side wall of the camera navigation box, the third insert positioned on the third side wall of the camera navigation box, and the fourth insert positioned on the base of the camera navigation box;
a computing device configured to:
generate a digital environment corresponding to the training environment, wherein the digital environment comprises a plurality of computer-generated elements,
track a current position of the simulated laparoscope with respect to the training environment by processing image data captured by the simulated laparoscope including one or more markers of the plurality of markers, and wherein the tracking is repeated after a pre-determined period of time,
calculate positional information of the simulated laparoscope from the processed image data,
update the digital environment using the calculated positional information of the simulated laparoscope wherein at least part of the calculated positional information of the simulated laparoscope is represented using a cursor in the digital environment,
monitor user performance of a camera navigation exercise by comparing the calculated positional information of the simulated laparoscope with stored information of one or more of the plurality of computer-generated elements of the digital environment, and
generate feedback based on the monitored user performance of the camera navigation exercise; and
a monitor configured to display the digital environment and the generated feedback.

2. The system of claim 1, wherein each of the plurality of markers are unique from one another, wherein the plurality of markers are arranged in a checkerboard pattern having light squares and alternating dark squares on each of the first, second, third and fourth inserts, and wherein the plurality of markers are QR codes.

3. The system of claim 2, wherein information about each of the plurality of markers is stored in a memory storage device connected to the computing device, wherein the information about each of the plurality of markers includes coordinates based on a pre-determined reference point for each of the plurality of markers to pinpoint a location of each of the plurality of markers with respect to the training environment.

4. The system of claim 3, wherein the computing device configured to update the digital environment is configured to update the digital environment 60 times per second.

5. The system of claim 4, wherein one of the plurality of computer-generated elements is a virtual menu and wherein the computing device configured to compare the calculated positional information of the simulated laparoscope with a location of the virtual menu to identify a user selection of a portion of the virtual menu and a user selection of the camera navigation exercise.

6. The system of claim 5, wherein the updating of the digital environment includes the computing device configured to retrieve predefined computer-generated elements associated with the user selection of the camera navigation exercise from the memory storage device, and to update the digital environment with the predefined computer-generated elements.

7. The system of claim 6, wherein information about the predefined computer-generated elements is stored in the memory storage device, and the computing device is configured to retrieve the information about the predefined computer-generated elements from the memory storage device when the computing device identifies the user selection of the camera navigation exercise.

8. The system of claim 7, wherein a first one of the predefined computer-generated elements is a virtual button and wherein the computing device is configured to compare the calculated positional information of the simulated laparoscope with a location of the virtual button to determine a user selection of the virtual button.

9. The system of claim 8, wherein a second one of the predefined computer-generated elements is a virtual meter, the computing device is configured to compare the calculated positional information of the simulated laparoscope with locations of the predefined computer-generated elements to quantify a user performance of the camera navigation exercise, and the computing device is configured to update the digital environment with the virtual meter to represent the quantified user performance of the camera navigation exercise.

10. The system of claim 9, wherein the computing device is configured to require a minimum of four unique markers from the plurality of markers to be captured by the simulated laparoscope, and the computing device is configured to calculate positional information of the simulated laparoscope from the processed image data including the four unique markers.

11. The system of claim 10, wherein the computing device is configured to transform the image data captured by the simulated laparoscope into a greyscale binary image, the computing device is configured to determine what portions of the greyscale binary image are “on” on a pixel-by-pixel basis, and the computing device is configured to filter the greyscale binary image to only include portions that have a pre-determined shape corresponding to a shape of the plurality of markers stored in memory.

12. The system of claim 11, wherein the computing device is configured to remove a distortion from the image data captured by the simulated laparoscope by calculating a translation, rotation, scaling, or skewing of the image data, and the computing device is configured to transform images of the plurality of markers within the image data to correspond to a pre-determined shape associated with each of the plurality of markers using the calculated translation, rotation, scaling or skewing of the image data.

13. The system of claim 12, wherein the third side wall includes a hinge connecting the base to the third side wall, and the third side wall is attached to the first side wall and the second side wall via complimentary hooks and slits.

14. The system of claim 13, wherein the simulated laparoscope comprises a camera and a rotary sensor and the computing device is configured to measure a rotational angle of the simulated laparoscope relative to a horizon via the rotary sensor.

15. The system of claim 14, wherein the camera navigation exercise is a tube targeting exercise and the computing device is configured to require maneuvering of a perspective of the simulated laparoscope and the cursor to center about a virtual target, the virtual target being planar and aligned with a viewing surface representing the horizon within the digital environment, and a virtual tube extending perpendicularly from the virtual target, the virtual target being a third one of the predefined computer-generated elements and the virtual tube being a fourth one of the predefined computer-generated elements.

16. The system of claim 15, wherein the computing device is configured to detect a collision between the cursor and the virtual tube, the virtual tube configured to obstruct the virtual target from the cursor.

17. The system of claim 16, wherein the collision is detected by the computing device configured to calculate a path from a current location of the cursor to the virtual target and to identify points on the path intersecting with the virtual tube.

18. The system of claim 17, wherein the computing device is configured to determine and to generate rotational feedback of how much and how often the simulated laparoscope is rotated over a period of time during the tube targeting exercise and rotational directions of the simulated laparoscope rotated over the period of time and wherein the monitor is configured to display the generated rotational feedback.

19. The system of claim 18, wherein the computing device is configured to measure changes in a viewing distance between the simulated laparoscope and the training environment from the calculated positional information of the simulated laparoscope, the computing device is configured to generate a viewing distance feedback including the measured changes in the viewing distance over the period of time, and the monitor is configured to display the generated viewing distance feedback.

20. The system of claim 14, wherein the computing device is configured to compare a path traveled by the cursor within the digital environment to a plurality of path segments along a pre-determined path to determine an amount of time the cursor is inside the pre-determined path compared to an amount of time the cursor is outside of the pre-determined path.
